(19) United States
(12) Patent Application Publication
(10) Pub. No.: US 2024/0285398 A1
(43) Pub. Date: Aug. 29, 2024

(54) LEAFLET ASSEMBLIES AND METHODS FOR ATTACHING LEAFLETS TO A FRAME OF AN EXPANDABLE PROSTHETIC HEART VALVE

(71) Applicant: EDWARDS LIFESCIENCES CORPORATION, Irvine, CA (US)

(72) Inventors: Tamir S. Levi, Zikhron Yaakov (IL); Elena Sherman, Pardes Hana (IL); Shir Lazovski, Evron kibutz (IL); Ziv Yohanan, Kfar Hahoresh (IL); Nikolai Gurovich, Hadera (IL); Bich Hoang Pham, Irvine, CA (US)

(21) Appl. No.: 18/656,929

(22) Filed: May 7, 2024

Related U.S. Application Data
(63) Continuation of application No. PCT/US2022/049666, filed on Nov. 11, 2022.
(60) Provisional application No. 63/343,359, filed on May 18, 2022.
(60) Provisional application No. 63/300,302, filed on Jan. 18, 2022.
(60) Provisional application No. 63/278,922, filed on Nov. 12, 2021.

(51) Int. Cl. A61F 2/24

(52) U.S. Cl. CPC A61F 2/2415; A61F 2/2418; A61F 2220/0075

(57) ABSTRACT

A prosthetic heart valve including a plurality of leaflets attached to the frame is disclosed. The plurality of leaflets is disposed within the frame and configured to regulate the flow of blood through the frame in one direction, each leaflet comprising a cusp edge portion. The prosthetic heart valve can further include at least one suture extending along a cusp edge portion of at least a first leaflet of the plurality of leaflets, a plurality of in-and-out stitches extending through the suture and the cusp edge portion of the first leaflet, and a plurality of whip stitches that extend around selected struts of the frame and the suture to couple the cusp edge portion of the first leaflet to the frame.

### CROSS-REFERENCE TO RELATED APPLICATIONS

This application is a continuation of PCT Application No. PCT/US2022/049666, filed Nov. 11, 2022, which claims the benefit of U.S. Provisional Patent Application Nos. 63/278,922, filed Nov. 12, 2021, 63/300,302, filed Jan. 18, 2022, and 63/343,359, filed May 18, 2022, the entire contents of each of which are incorporated by reference herein.

### FIELD

The present disclosure relates to prosthetic heart valves and systems and methods for securing leaflets to a frame of a prosthetic heart valve.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

Most expandable, transcatheter heart valves comprise a radially expandable and compressible cylindrical metal frame and prosthetic leaflets attached to an inside the frame via an inner skirt. In such configurations, cusp edge portions of the leaflets can be sutured to the fabric of the inner skirt and the inner skirt can then be sutured to an inside of the frame. Further, commissures can be formed by connecting pairs of commissure tabs of adjacent leaflets to each other and to commissure windows formed in the frame.

### SUMMARY

Described herein are prosthetic heart valves, delivery apparatus, and methods for assembling and implanting prosthetic heart valves. The disclosed prosthetic heart valves, delivery apparatus, and methods can, for example, provide easier and faster means for assembling leaflets to a frame of the prosthetic heart valves. As such, the devices and methods disclosed herein can, among other things, overcome one or more of the deficiencies of typical prosthetic heart valves.

A prosthetic heart valve can comprise a frame and a valve structure coupled to the frame. In addition to these components, a prosthetic heart valve can further comprise one or more of the components disclosed herein.

In some examples, a prosthetic heart valve can comprise a sealing member configured to reduce paravalvular leakage.

In some examples, a prosthetic heart valve can comprise a plurality of leaflets, where each leaflet comprises a cusp edge portion, at least one suture extending along a cusp edge portion of at least a first leaflet of the plurality of leaflets, and a plurality of in-and-out stitches extending through the suture and the cusp edge portion of the first leaflet.

In some examples, a prosthetic heart valve can comprise a plurality of leaflets forming a leaflet assembly, a connecting suture line extending along the cusp edge portion of each leaflet and securing the plurality of leaflets together, the connecting suture line including one or more first sutures arranged along the cusp edge portion on at least one side of each leaflet and one or more second sutures stitched in a running-stitch pattern through the one or more first sutures and through each leaflet, and a plurality of wedge members formed from the one or more first sutures, each wedge member extending from the cusp edge portions of two adjacent leaflets of the plurality of leaflets and between two commissure tabs of the two adjacent leaflets.

In some examples, a prosthetic heart valve comprises a frame comprising a plurality of interconnected struts; a plurality of leaflets disposed within the frame and configured to regulate a flow of blood through the frame in one direction, each leaflet comprising a cusp edge portion; and at least one suture extending along a cusp edge portion of at least a first leaflet of the plurality of leaflets, a plurality of in-and-out stitches extending through the suture and the cusp edge portion of the first leaflet, and a plurality of whip stitches that extend around selected struts of the plurality of the interconnected struts and the suture to couple the cusp edge portion of the first leaflet to the frame.

In some examples, a leaflet assembly for a prosthetic heart valve comprises a plurality of leaflets, each leaflet comprising opposing commissure tabs disposed on opposite sides of the leaflet and a cusp edge portion extending between the opposing commissure tabs. The leaflet assembly further comprises a connecting suture line extending along the cusp edge portion of each leaflet and securing the plurality of leaflets together, the connecting suture line including one or more first sutures arranged along the cusp edge portion on at least one side of each leaflet and one or more second sutures stitched in a running-stitch pattern through the one or more first sutures and through each leaflet. The leaflet assembly further comprises a plurality of wedge members formed from the one or more first sutures, each wedge member extending from the cusp edge portions of two adjacent leaflets of the plurality of leaflets and between two commissure tabs of the two adjacent leaflets.

In some examples, a prosthetic heart valve comprises a frame comprising a plurality of interconnected struts and a plurality of commissure supports defining respective commissure windows and a plurality of leaflets. Each leaflet comprises opposing commissure tabs disposed on opposite sides of the leaflet and a cusp edge portion extending between the opposing commissure tabs. The plurality of leaflets are connected together and to the frame by a connecting suture line extending along the cusp edge portions of the leaflets, the connecting suture line comprising one or more first sutures arranged along the cusp edge portion on at least one side of each leaflet and one or more second sutures stitched in an in-and-out pattern through the leaflets and the one or more first sutures. The prosthetic heart valve further comprises a plurality of commissures secured to the plurality of commissure supports, each commissure comprising two commissure tabs, one from each of two adjacent leaflets of the plurality of leaflets, and a wedge member extending between and along the two commissure tabs on an exterior of a corresponding commissure support of the frame, the wedge member comprising two or more overlapping portions of the one or more first sutures of the connecting suture line.

In some examples, a prosthetic heart valve comprises a frame comprising a plurality of interconnected struts and a plurality of commissure supports defining respective commissure windows. The prosthetic heart valve further comprises a plurality of leaflets, each leaflet comprising opposing commissure tabs disposed on opposite sides of the leaflet and a cusp edge portion extending between the opposing commissure tabs, where the plurality of leaflets is connected together and to the frame by a connecting suture line extending along the cusp edge portions of the leaflets. The connecting suture line comprises one or more first sutures arranged along the cusp edge portion on at least one side of each leaflet and one or more second sutures stitched in an in-and-out pattern through the leaflets and the one or more first sutures. The prosthetic heart valve further comprises a plurality of commissures secured to the plurality of commissure supports, each commissure comprising two commissure tabs, one from each of two adjacent leaflets of the plurality of leaflets, extending through a corresponding commissure window and exterior to the frame, and a wedge member extending between and along inner surfaces of the two commissure tabs, the wedge member comprising two or more overlapping suture segments of the one or more first sutures of the connecting suture line. The wedge member is secured to the two commissure tabs with a plurality of stitches extending through the wedge member and a plurality of in-and-out stitches that extend along the inner surfaces of the two commissure tabs.

In some examples, a prosthetic heart valve comprises a frame comprising a plurality of interconnected struts and a plurality of commissure supports defining respective commissure windows. The prosthetic heart valve further comprises a plurality of leaflets, each leaflet comprising opposing commissure tabs disposed on opposite sides of the leaflet and a cusp edge portion extending between the opposing commissure tabs, where the plurality of leaflets is connected together and to the frame by a connecting suture line extending along the cusp edge portions of the leaflets. The connecting suture line comprises a plurality of first sutures arranged along the cusp edge portion on both sides of each leaflet and one or more second sutures stitched in an in-and-out pattern through the leaflets and the plurality of first sutures. The prosthetic heart valve further comprises a plurality of commissures secured to the plurality of commissure supports, each commissure comprising two commissure tabs, one from each of two adjacent leaflets of the plurality of leaflets, extending through a corresponding commissure window, and a wedge member extending between and along the two commissure tabs, the wedge member formed from two end portions of the plurality of first sutures and comprising three or more overlapping segments of the two end portions.

In some examples, a prosthetic heart valve comprises one or more of the components recited in Examples 1-37 and 74-91 below.

A method can comprise securing a plurality of leaflets of a prosthetic heart valve together into a leaflet assembly and securing adjacent commissure tabs of adjacent leaflets of the plurality of leaflets to a frame of the prosthetic heart valve.

In some examples, a method can comprise attaching a connecting suture line formed along cusp edge portions of a plurality of leaflets of a leaflet assembly to struts of a frame of a prosthetic heart valve

In some examples, a method can comprise attaching pairs of commissure tabs of adjacent leaflets of a leaflet assembly of a prosthetic heart valve to commissure windows of a frame of the prosthetic heart valve by extending each pair of commissure tabs radially through a corresponding commissure window formed by a corresponding commissure support portion of the frame and securing a wedge member integrated with a connecting suture line of the leaflet assembly to the pair of commissure tabs on an exterior of the commissure window.

In some examples, a method for forming a leaflet assembly for a prosthetic heart valve comprises securing a plurality of leaflets together into a leaflet assembly, off a frame of the prosthetic heart valve, by arranging one or more sutures along a cusp edge portion on at least one side of each leaflet of the plurality of leaflets and extending a plurality of in-and-out stitches through the one or more sutures and the plurality of leaflets, along the cusp edge portion of each leaflet. The method further comprises pairing each commissure tab of each leaflet of the plurality of leaflets with an adjacent commissure tab of an adjacent leaflet, and for each pair of commissure tabs, extending a portion of the one or more sutures between the pair of commissure tabs and overlapping the portion with itself to form an integrated wedge member with a thickness that is greater than a thickness of the one or more sutures.

In some examples, a method comprises attaching a connecting suture line formed along cusp edge portions of a plurality of leaflets of a leaflet assembly to struts of a frame of a prosthetic heart valve by extending a plurality of whip stitches around or through at least a portion of the connecting suture line and around selected struts of the frame to secure the cusp edge portions of the leaflets directly to the frame without extending the whip stitches through a tissue of the leaflets. The connecting suture line comprises a suture disposed on the cusp edge portion of at least one side of each leaflet of the plurality of leaflets and a plurality of in-and-out stitches extending through the suture and the cusp edge portion of each leaflet. The method further comprises attaching pairs of commissure tabs of adjacent leaflets of the leaflet assembly to commissure windows of the frame by extending each pair of commissure tabs radially through a corresponding commissure window formed by a corresponding commissure support portion of the frame and securing a wedge member integrated with the connecting suture line to the pair of commissure tabs on an exterior of the commissure window, the wedge member formed by two or more overlapping portions of the suture.

In some examples, a method comprises attaching a connecting suture line formed along cusp edge portions of a plurality of leaflets of a leaflet assembly to struts of a frame of a prosthetic heart valve. The connecting suture line comprises one or more sutures disposed on the cusp edge portion of at least one side of each leaflet of the plurality of leaflets and a plurality of in-and-out stitches extending through the suture and the cusp edge portion of each leaflet. The method further comprises attaching pairs of commissure tabs of adjacent leaflets of the leaflet assembly to commissure windows of the frame by extending each pair of commissure tabs radially through a corresponding commissure window formed by a corresponding commissure support portion of the frame, where the plurality of in-and-out stitches further extend along each commissure tab of each pair of commissure tabs. The method further comprises forming a wedge member between commissure tabs of each pair of commissure tabs from two or more overlapping portions of the one or more sutures of the connecting suture line and securing each wedge member between the commissure tabs of each corresponding pair of commissure tabs with a plurality of stitches extending through the wedge member and the plurality of in-and-out stitches along an inner surface of each commissure tab of the corresponding pair of commissure tabs to form a commissure.

In some examples, a method comprises attaching a connecting suture line formed along cusp edge portions of a plurality of leaflets of a leaflet assembly to struts of a frame of a prosthetic heart valve, where the connecting suture line comprises one or more sutures disposed on the cusp edge portion of at least one side of each leaflet of the plurality of leaflets and a plurality of in-and-out stitches extending through the suture and the cusp edge portion of each leaflet. The method further comprises attaching pairs of commissure tabs of adjacent leaflets of the leaflet assembly to commissure windows of the frame by extending each pair of commissure tabs radially through a corresponding commissure window formed by a corresponding commissure support portion of the frame. The method further comprises forming a wedge member between commissure tabs of each pair of commissure tabs from two or more overlapping portions of the one or more sutures of the connecting suture line and securing each wedge member to a first end of the corresponding support portion, above the corresponding commissure window, to a second end of the corresponding support portion, below the corresponding commissure window, and to the commissure tabs to form a commissure.

In some examples, a method comprises one or more of the features recited in Examples 38-73 and 92-98 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a perspective view of a prosthetic heart valve, according to an example.

FIG. 2 is a side view of an example of a delivery apparatus configured to deliver and implant a radially expandable prosthetic heart valve at an implantation site.

FIG. 3 is a perspective view of a prosthetic heart valve, according to an example.

FIG. 4 is a plan view of a leaflet assembly in a flat configuration with leaflets of the leaflet assembly secured together with a connecting suture line disposed along a cusp edge portion of each of the leaflets.

FIG. 5 is a perspective view of the leaflet assembly of FIG. 4.

FIG. 6 is a plan view of one leaflet with a connecting suture line secured to a cusp edge portion of the leaflet.

FIG. 7 is a plan view of an outflow end of a prosthetic heart valve comprising the leaflet assembly of FIG. 4 attached to an inside of a frame of the prosthetic heart valve.

FIG. 8 is an interior, perspective view of a portion of an inflow end of the prosthetic heart valve of FIG. 7 showing the connecting suture line of the leaflet assembly secured directly to struts of the frame.

FIG. 9A is an exterior, perspective view of a portion of the inflow end of the prosthetic heart valve of FIG. 7 showing the connecting suture line of the leaflet assembly secured directly to struts of the frame.

FIG. 9B is a schematic of a cross-sectional view of a leaflet connected to the frame of FIG. 9A using the connecting suture line of the leaflet assembly.

FIG. 9C is a schematic of a portion of the connecting suture line of the leaflet assembly and a whip stitch connected to the connecting suture line for connecting the leaflet assembly to the frame of FIG. 9A

FIG. 9D is a schematic showing the connecting suture wrapped around a strut of the frame of FIG. 9A and around a portion of the connecting suture line of a leaflet of the leaflet assembly.

FIG. 10 is a perspective view of a portion of the prosthetic heart valve of FIG. 7 showing an attachment of an outer skirt to an outer surface of the frame.

FIG. 11 is another perspective view of the portion of the prosthetic heart valve of FIG. 7 showing the attachment of the outer skirt to the outer surface of the frame and an attachment interface of the leaflet assembly to the frame.

FIG. 12 is another perspective view of the prosthetic heart valve of FIG. 7 showing the attachment of the outer skirt to the outer surface of the frame, the outer skirt comprising multiple skirt portions.

FIG. 13A is a perspective view of the prosthetic heart valve of FIG. 7 showing an example of a method for attaching an outer skirt comprising a connecting suture line and the leaflet assembly comprising another connecting suture line to the frame using a common connecting suture.

FIG. 13B is another perspective view, from an end of the frame of the prosthetic heart valve of FIG. 13A, showing the simultaneous connection of the outer skirt and the leaflet assembly to the frame using the respective connecting suture lines on the outer skirt and the leaflet assembly.

FIG. 14 shows a conventional configuration for a commissure of a prosthetic heart valve which includes a separate wedge member arranged between commissure tabs of two adjacent leaflets that extend through a commissure window of a frame of the prosthetic heart valve, the wedge member configured to prevent the commissure tabs from pulling back through the commissure window.

FIG. 15 shows an example of a commissure wedge member integrated with a connecting suture line disposed along the cusp edge portions of leaflets of a leaflet assembly and formed as a loop.

FIG. 16 shows an example of a commissure wedge member integrated with a connecting suture line disposed along the cusp edge portions of leaflets of a leaflet assembly and formed as a single wedge member.

FIG. 17 is a plan view of a leaflet and a commissure wedge member formed from overlapping portions of a thicker suture of a connecting suture line configured to be connected along a cusp edge portion of the leaflet.

FIG. 18A is a detail view of a portion of the leaflet of FIG. 17 showing the commissure wedge member arranged along a commissure tab of the leaflet.

FIG. 18B is a side view of an example of the wedge member of FIG. 18A, where an additional, distinguishing suture extends through the wedge member.

FIG. 19A is a plan view of two adjacent leaflets of a leaflet assembly in a flat configuration with a commissure wedge member formed from overlapping portions of a main suture disposed along adjacent commissure tabs of the two adjacent leaflets and a connecting suture line formed along the cusp edge portions of the two adjacent leaflets using main sutures from the two adjacent leaflets and a thinner suture stitched in an in-and-out pattern along the cusp edge portions.

FIG. 19B is a plan view of two adjacent leaflets of a leaflet assembly in a flat configuration with a commissure wedge member formed from overlapping portions of a main suture disposed along adjacent commissure tabs of the two adjacent leaflets and a connecting suture line formed along the cusp edge portions of the two adjacent leaflets using the main suture and a thinner suture stitched in an in-and-out pattern along the cusp edge portions.

FIG. 20 shows a portion of a method for attaching a pair of commissure tabs of the leaflet assembly of FIG. 19 to a commissure window of a frame of a prosthetic heart valve which includes passing the commissure tabs through the commissure window and extending the commissure wedge member from the cusp edge portions of the leaflets and along the commissure tabs on an outside of the frame.

FIGS. 21 and 22 show a portion of the method for attaching a pair of commissure tabs of the leaflet assembly of FIG. 19 to a commissure window of a frame of a prosthetic heart valve which includes securing the commissure wedge member to an upper portion of the commissure window.

FIG. 23 shows a portion of the method for attaching a pair of commissure tabs of the leaflet assembly of FIG. 19 to a commissure window of a frame of a prosthetic heart valve which includes securing the commissure tabs to the commissure wedge member.

FIGS. 24 and 25 show a portion of the method for attaching a pair of commissure tabs of the leaflet assembly of FIG. 19 to a commissure window of a frame of a prosthetic heart valve which includes securing the commissure wedge member to a lower portion of the commissure window.

FIG. 26 shows a portion of the method for attaching a pair of commissure tabs of the leaflet assembly of FIG. 19 to a commissure window of a frame of a prosthetic heart valve which includes further stitching the commissure tabs to each other and around the commissure wedge member.

FIG. 27 shows a portion of the method for attaching a pair of commissure tabs of the leaflet assembly of FIG. 19 to a commissure window of a frame of a prosthetic heart valve which includes forming a final upper knot at a top of the formed commissure.

FIGS. 28-30 show a portion of the method for attaching a pair of commissure tabs of the leaflet assembly of FIG. 19 to a commissure window of a frame of a prosthetic heart valve which includes threading loose strands of the suture used to form the commissure through one or more portions of the commissure to form the final commissure.

FIG. 31 is a perspective view of an example of a prosthetic heart valve comprising an outer skirt disposed around an outer surface of a frame of the prosthetic heart valve and secured to integrated commissure wedge members of commissures of the prosthetic heart valve.

FIG. 32 is a perspective view of an example of a prosthetic heart valve comprising an outer skirt disposed around an outer surface of a frame of the prosthetic heart valve and secured to integrated commissure wedge members of commissures of the prosthetic heart valve.

FIG. 33 shows a portion of a method for attaching a pair of a commissure tabs of a leaflet assembly to a commissure window of a frame of a prosthetic heart valve which includes passing the pair of commissure tabs through the commissure window of the frame with suture end portions of main sutures of a connecting suture line formed along the cusp edge portions of the leaflets of the leaflet assembly extending away from the connecting suture line within the frame.

FIGS. 34 and 35 show a portion of the method for attaching a pair of a commissure tabs of a leaflet assembly to a commissure window of a frame of a prosthetic heart valve which includes forming an integrated wedge member across and between the commissure tabs using the suture end portions from the connecting suture line.

FIGS. 36-38 show a portion of the method for attaching a pair of a commissure tabs of a leaflet assembly to a commissure window of a frame of a prosthetic heart valve which includes securing the wedge member to a plurality of in-and-out stitches extending along inner surfaces of the commissure tabs with one or more additional sutures and forming a knot with the one or more additional sutures at an inlet end of the commissure tabs.

FIGS. 39 and 40 show a portion of the method for attaching a pair of a commissure tabs of a leaflet assembly to a commissure window of a frame of a prosthetic heart valve which includes closing the commissure tabs around the wedge member with one or more sutures tied around outer surfaces of the commissure tabs.

FIGS. 41-43 show a portion of the method for attaching a pair of a commissure tabs of a leaflet assembly to a commissure window of a frame of a prosthetic heart valve which includes threading end portions of the one or more sutures used to close the commissure tabs through an interior of the commissure to result in the completed commissure secured to the frame.

FIGS. 44-50 show a portion of a method for attaching a pair of a commissure tabs of a leaflet assembly to a commissure window of a frame of a prosthetic heart valve with an integrated wedge member which includes securing the integrated wedge member to the commissure tabs using an alternating stitch pattern that extends two opposing sutures through the wedge member and opposing in-and-out stitches in the commissure tabs, thereby forming a plurality of loops along the wedge member between the commissure tabs which secure the wedge member to the commissure tabs.

FIGS. 51-53 show a portion of the method for attaching a pair of a commissure tabs of a leaflet assembly to a commissure window of a frame of a prosthetic heart valve with an integrated wedge member which includes passing suture tails of the two opposing sutures through the wedge member at an outflow end of the commissure tabs and forming a knot with the suture tails.

FIG. 54 show an interior view of the frame of the prosthetic heart valve with the suture tails of the two opposing sutures extending in opposite directions above the commissure tabs proximate to an outflow end of the commissure window.

FIGS. 55 and 56 show a portion of the method for attaching a pair of a commissure tabs of a leaflet assembly to a commissure window of a frame of a prosthetic heart valve with an integrated wedge member which includes passing one of the suture tails of the two opposing sutures through a knot formed at an inflow end of the commissure tabs.

FIGS. 57-59 show a portion of the method for attaching a pair of a commissure tabs of a leaflet assembly to a commissure window of a frame of a prosthetic heart valve with an integrated wedge member which includes folding the commissure tabs toward one another and extending the two opposing sutures through in-and-out stitches on outer surfaces of the commissure tabs to close the commissure tabs around the wedge member.

FIG. 60 is a plan view of a leaflet assembly in a flat configuration with leaflets of the leaflet assembly secured together with a connecting suture line disposed along a cusp edge portion of each of the leaflets, the connecting suture line formed from a single suture extending along the cusp edge portion on a first side of all three leaflets and three individual sutures extending along the cusp edge portion on a second side of the three leaflets, each of the three individual sutures extending along the cusp edge portion of a different leaflet.

FIG. 61 is a plan view of two adjacent leaflets of the leaflet assembly of FIG. 60 in a flat configuration showing the connecting suture line along the first side of the two adjacent leaflets.

FIG. 62 is a plan view of two adjacent leaflets of the leaflet assembly of FIG. 60 in a flat configuration showing the connecting suture line along the second side of the two adjacent leaflets and two suture end portions formed at the adjacent commissure tabs of the two adjacent leaflets.

FIG. 63 shows a portion of a method for attaching a pair of a commissure tabs of a leaflet assembly to a commissure window of a frame of a prosthetic heart valve which includes passing the pair of commissure tabs through the commissure window of the frame and extending suture end portions of main sutures of a connecting suture line formed along the cusp edge portions of the leaflets of the leaflet assembly across and between the pair of commissure tabs, on an exterior of the frame, and toward an outflow end of the frame.

FIG. 64 shows a portion of the method for attaching the pair of a commissure tabs of the leaflet assembly to the commissure window of the frame of the prosthetic heart valve which includes tying the suture end portions to an outflow end of a commissure support forming the commissure window.

FIG. 65 shows a portion of the method for attaching the pair of a commissure tabs of the leaflet assembly to the commissure window of the frame of the prosthetic heart valve which includes extending one of the suture end portions back across and between the pair of commissure tabs to form a wedge member that is three suture-widths thick.

FIG. 66 shows a portion of the method for attaching the pair of a commissure tabs of the leaflet assembly to the commissure window of the frame of the prosthetic heart valve which includes tying one or both of the suture end portions to an inflow end of the commissure support forming the commissure window and closing the pair of commissure tabs around the wedge member to form a commissure.

FIG. 67 shows a portion of the method for attaching the pair of a commissure tabs of the leaflet assembly to the commissure window of the frame of the prosthetic heart valve which includes trimming free ends of the suture end portions to form the final commissure.

FIG. 68 is a plan view of two adjacent leaflets of a leaflet assembly in a flat configuration showing a connecting suture line formed along the cusp edge portions of the two adjacent leaflets, where the connecting suture line is formed from individual sutures extending along the cusp edge portion of a corresponding leaflet, and where each individual suture is folded over one end of the cusp edge portion with portions extending along both sides of the corresponding leaflet such that two suture end portions are formed at an opposite end of the cups edge portion.

FIG. 69 is a cross-sectional view of one of the two adjacent leaflets of FIG. 68 showing the connecting suture line formed along a portion of the leaflet.

FIG. 70 is a perspective view of a portion of a leaflet assembly showing a method for forming a wedge member from two suture end portions extending from a connecting suture line of the leaflet assembly, wherein the suture end portions are stitched together to form a thicker portion in the wedge member.

FIG. 71 shows an example of the wedge member of FIG. 70 being secured to a pair of commissure tabs extending through a commissure window of a prosthetic heart valve frame and being secured to a commissure support of the frame that forms the commissure window.

FIG. 72 shows an example of one or more stitches used to secure a wedge member between and to a pair of commissure tabs on an exterior of a commissure window of a frame of a prosthetic heart valve.

### DETAILED DESCRIPTION

### General Considerations

For purposes of this description, certain aspects, advantages, and novel features of the examples of this disclosure are described herein. The described methods, systems, and apparatus should not be construed as limiting in any way. Instead, the present disclosure is directed toward all novel and non-obvious features and aspects of the various disclosed examples, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present, or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,”“an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

As used herein, “e.g.” means “for example,” and “i.e.” means “that is.”

### Examples of the Disclosed Technology

Described herein are examples of radially expandable and compressible prosthetic heart valves including an annular frame. In some examples, the frame of the prosthetic heart valve can include a plurality of rows of cells formed by interconnected struts of the frame. The plurality of rows of cells can be formed between an inflow end and an outflow end of the frame.

The prosthetic heart valve may further include a plurality of leaflets attached to the frame. As introduced above, in most prosthetic heart valves, the leaflets are attached to an inside of the frame via an inner skirt. In such configurations, cusp edge portions of the leaflets can be sutured to the fabric of the inner skirt and the inner skirt can then be sutured to the inside of the frame. The leaflets can be attached to the frame via commissures formed by joining pairs of adjacent ends (e.g., commissure tabs) of the leaflets and connecting the joined pairs of adjacent ends to commissure windows formed in the frame. In some examples, separate wedge members can be used to prevent the commissures from passing back through the commissure windows by securing each wedge member to a corresponding pair of commissure tabs, on an exterior of the frame. However, such assembly techniques can take significant time and effort, thereby increasing production time and costs.

As disclosed herein, in some examples, one or more leaflets of the prosthetic heart valve can comprise a connecting suture line along a cusp edge portion of the leaflet. The connecting suture line can be attached to the leaflet off of the frame. The connecting suture line can be further configured to enable direct attachment of the cusp edge portion of the leaflet to a portion of struts of the frame, without being attached to an inner skirt and without extending additional sutures through the cusp edge portion of the leaflets.

In some examples, one or more sutures of the connecting suture line can extend from cusp edge portions of adjacent leaflets of a leaflet assembly and between a pair of commissure tabs of the adjacent leaflets to form a commissure wedge member. After passing the pair of commissure tabs through a commissure window formed by a commissure support portion of the frame, the wedge member can be secured to the pair of commissure tabs on an outside of the frame, thereby forming a commissure that can prevent the commissure tabs from being pulled back through the commissure window and securing the leaflets to the frame.

Prosthetic valves disclosed herein can be radially compressible and expandable between a radially compressed state and a radially expanded state. Thus, the prosthetic valves can be crimped on or retained by an implant delivery apparatus in the radially compressed state during delivery, and then expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

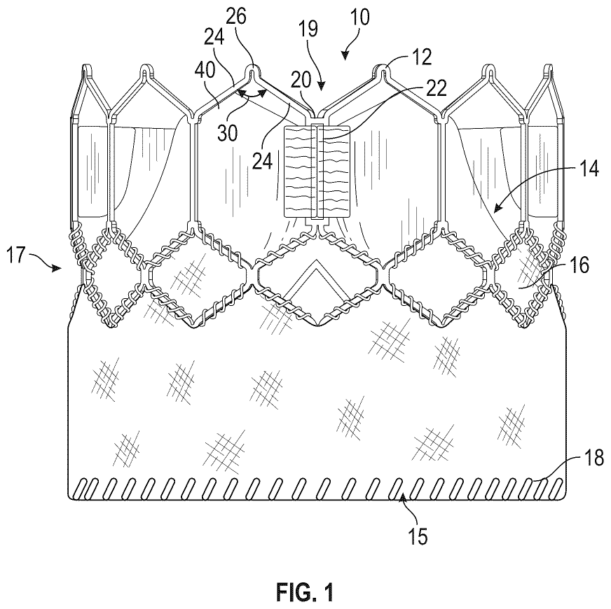

FIG. 1 shows an exemplary prosthetic valve 10, according to one example. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In some examples, the disclosed prosthetic valves can be implanted within a docking device implanted within or at the native mitral valve, such as disclosed in PCT Publication No. WO2020/247907, which is incorporated herein by reference. In some examples, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

The prosthetic valve 10 comprises four main components: a stent or frame 12, a valvular structure 14, an inner skirt 16, and a perivalvular outer sealing member or outer skirt 18. The prosthetic valve 10 can have an inflow end portion 15, an intermediate portion 17, and an outflow end portion 19. The inner skirt 16 can be arranged on and/or coupled to an inner surface of the frame 12, while the outer skirt 18 can be arranged on and/or coupled to an outer surface of the frame 12.

The valvular structure 14 can comprise three leaflets 40, collectively forming a leaflet structure, which can be arranged to collapse in a tricuspid arrangement, although in other examples there can be greater or fewer number of leaflets (e.g., one or more leaflets 40). The leaflets 40 can be secured to one another at their adjacent sides to form commissures 22 of the leaflet structure 14. The lower edge of valvular structure 14 can have an undulating, curved scalloped shape and can be secured to the inner skirt 16 by sutures (not shown). In some examples, the leaflets 40 can be formed of pericardial tissue (e.g., bovine pericardial tissue), biocompatible synthetic materials, or various other suitable natural or synthetic materials as known in the art and described in U.S. Pat. No. 6,730,118, which is incorporated by reference herein.

The frame 12 can be radially compressible (collapsible) and expandable (e.g., expanded configuration shown in FIG. 1) and comprise a plurality of interconnected struts 24. A plurality of apices 26 that are spaced circumferentially apart are formed at the inflow end portion 15 and the outflow end portion 19 of the frame 12 (only the apices 26 at the outflow end portion 19 are visible in FIG. 1). Each apex 26 is formed at a junction between two angled struts 24 at either the inflow end portion 15 or the outflow end portion 19. FIG. 1 depicts a known frame design with apices 26 that form a U-shaped bend between the two angled struts 24. In some examples, an angle 30 between the two angled struts 24, connected at the apex 26, can be in a range of 90 to 120 degrees.

The frame 12 can be formed with a plurality of circumferentially spaced slots, or commissure windows 20 that are adapted to mount the commissures 22 of the valvular structure 14 to the frame. The frame 12 can be made of any of various suitable plastically-expandable materials (e.g., stainless steel, etc.) or self-expanding materials (e.g., Nitinol). When constructed of a plastically-expandable material, the frame 12 (and thus the prosthetic valve 10) can be crimped to a radially collapsed configuration on a delivery catheter or apparatus and then expanded inside a patient by an inflatable balloon or equivalent expansion mechanism. When constructed of a self-expandable material, the frame 12 (and thus the prosthetic valve 10) can be crimped to a radially collapsed configuration and restrained in the collapsed configuration by insertion into a sheath or equivalent mechanism of a delivery catheter. Once inside the body, the prosthetic valve can be advanced from the delivery sheath, which allows the prosthetic valve to expand to its functional size.

Suitable plastically-expandable materials that can be used to form the frames disclosed herein (e.g., the frame 12) include, metal alloys, polymers, or combinations thereof. Example metal alloys can comprise one or more of the following: nickel, cobalt, chromium, molybdenum, titanium, or other biocompatible metal. In some examples, the frame 12 can comprise stainless steel. In some examples, the frame 12 can comprise cobalt-chromium. In some examples, the frame 12 can comprise nickel-cobalt-chromium. In some examples, the frame 12 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight. Additional details regarding the prosthetic valve 10 and its various components are described in WIPO Patent Application Publication No. WO 2018/222799, which is incorporated herein by reference.

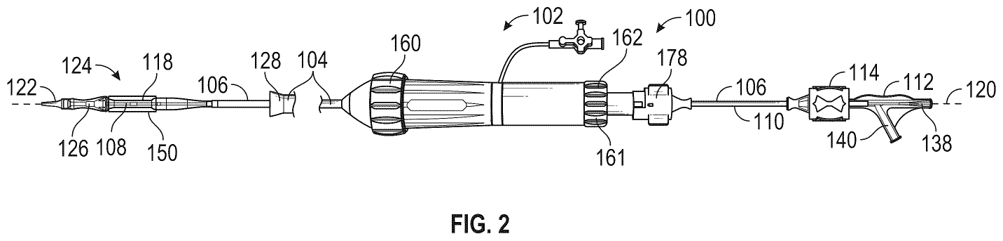

FIG. 2 shows a delivery apparatus 100, according to an example, that can be used to implant an expandable prosthetic heart valve (e.g., the prosthetic heart valve 10 of FIG. 1 and/or any of the other prosthetic heart valves described herein). In some examples, the delivery apparatus 100 is specifically adapted for use in introducing a prosthetic valve into a heart.

The delivery apparatus 100 in the illustrated example of FIG. 2 is a balloon catheter comprising a handle 102 and a steerable, outer shaft 104 extending distally from the handle 102. The delivery apparatus 100 can further comprise an intermediate shaft 106 (which also may be referred to as a balloon shaft) that extends proximally from the handle 102 and distally from the handle 102, the portion extending distally from the handle 102 also extending coaxially through the outer shaft 104. Additionally, the delivery apparatus 100 can further comprise an inner shaft 108 extending distally from the handle 102 coaxially through the intermediate shaft 106 and the outer shaft 104 and proximally from the handle 102 coaxially through the intermediate shaft 106.

The outer shaft 104 and the intermediate shaft 106 can be configured to translate (e.g., move) longitudinally, along a central longitudinal axis 120 of the delivery apparatus 100, relative to one another to facilitate delivery and positioning of a prosthetic valve at an implantation site in a patient's body.

The intermediate shaft 106 can include a proximal end portion 110 that extends proximally from a proximal end of the handle 102, to an adaptor 112. A rotatable knob 114 can be mounted on the proximal end portion 110 and can be configured to rotate the intermediate shaft 106 around the central longitudinal axis 120 and relative to the outer shaft 104.

The adaptor 112 can include a first port 138 configured to receive a guidewire therethrough and a second port 140 configured to receive fluid (e.g., inflation fluid) from a fluid source. The second port 140 can be fluidly coupled to an inner lumen of the intermediate shaft 106.

The intermediate shaft 106 can further include a distal end portion that extends distally beyond a distal end of the outer shaft 104 when a distal end of the outer shaft 104 is positioned away from an inflatable balloon 118 of the delivery apparatus 100. A distal end portion of the inner shaft 108 can extend distally beyond the distal end portion of the intermediate shaft 106.

The balloon 118 can be coupled to the distal end portion of the intermediate shaft 106.

In some examples, a distal end of the balloon 118 can be coupled to a distal end of the delivery apparatus 100, such as to a nose cone 122 (as shown in FIG. 2), or to an alternate component at the distal end of the delivery apparatus 100 (e.g., a distal shoulder). An intermediate portion of the balloon 118 can overlay a valve mounting portion 124 of a distal end portion of the delivery apparatus 100 and a distal end portion of the balloon 118 can overly a distal shoulder 126 of the delivery apparatus 100. The valve mounting portion 124 and the intermediate portion of the balloon 118 can be configured to receive a prosthetic heart valve in a radially compressed state. For example, as shown schematically in FIG. 2, a prosthetic heart valve 150 (which can be one of the prosthetic valves described herein) can be mounted around the balloon 118, at the valve mounting portion 124 of the delivery apparatus 100.

The balloon shoulder assembly, including the distal shoulder 126, is configured to maintain the prosthetic heart valve 150 (or other medical device) at a fixed position on the balloon 118 during delivery through the patient's vasculature.

The outer shaft 104 can include a distal tip portion 128 mounted on its distal end. The outer shaft 104 and the intermediate shaft 106 can be translated axially relative to one another to position the distal tip portion 128 adjacent to a proximal end of the valve mounting portion 124, when the prosthetic valve 150 is mounted in the radially compressed state on the valve mounting portion 124 (as shown in FIG. 2) and during delivery of the prosthetic valve to the target implantation site. As such, the distal tip portion 128 can be configured to resist movement of the prosthetic valve 150 relative to the balloon 118 proximally, in the axial direction, relative to the balloon 118, when the distal tip portion 128 is arranged adjacent to a proximal side of the valve mounting portion 124.

An annular space can be defined between an outer surface of the inner shaft 108 and an inner surface of the intermediate shaft 106 and can be configured to receive fluid from a fluid source via the second port 140 of the adaptor 112. The annular space can be fluidly coupled to a fluid passageway formed between the outer surface of the distal end portion of the inner shaft 108 and an inner surface of the balloon 118. As such, fluid from the fluid source can flow to the fluid passageway from the annular space to inflate the balloon 118 and radially expand and deploy the prosthetic valve 150.

An inner lumen of the inner shaft can be configured to receive a guidewire therethrough, for navigating the distal end portion of the delivery apparatus 100 to the target implantation site.

The handle 102 can include a steering mechanism configured to adjust the curvature of the distal end portion of the delivery apparatus 100. In the illustrated example, for example, the handle 102 includes an adjustment member, such as the illustrated rotatable knob 160, which in turn is operatively coupled to the proximal end portion of a pull wire. The pull wire can extend distally from the handle 102 through the outer shaft 104 and has a distal end portion affixed to the outer shaft 104 at or near the distal end of the outer shaft 104. Rotating the knob 160 can increase or decrease the tension in the pull wire, thereby adjusting the curvature of the distal end portion of the delivery apparatus 100. Further details on steering or flex mechanisms for the delivery apparatus can be found in U.S. Pat. No. 9,339,384, which is incorporated by reference herein.

The handle 102 can further include an adjustment mechanism 161 including an adjustment member, such as the illustrated rotatable knob 162, and an associated locking mechanism including another adjustment member, configured as a rotatable knob 178. The adjustment mechanism 161 is configured to adjust the axial position of the intermediate shaft 106 relative to the outer shaft 104 (e.g., for fine positioning at the implantation site). Further details on the delivery apparatus 100 can be found in PCT Application No. PCT/US2021/047056, which is incorporated by reference herein.

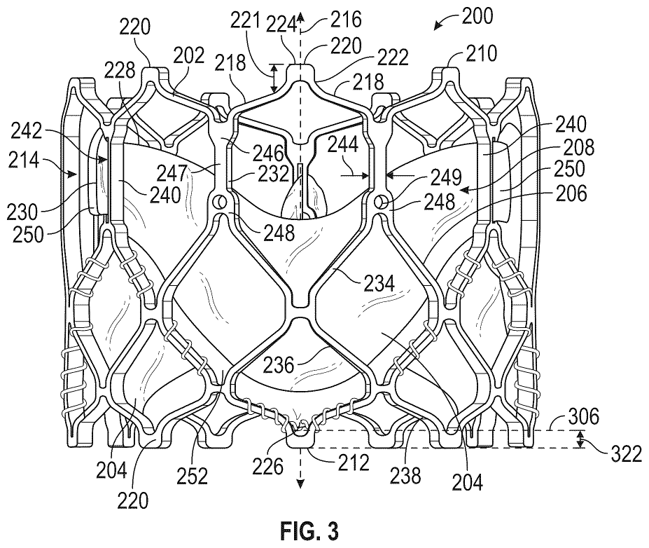

FIG. 3 shows an example of a prosthetic heart valve 200 comprising a radially expandable and compressible annular frame 202 and a plurality of leaflets 204 secured to the frame. Each leaflet 204 can comprise opposing commissure tabs 250 disposed on opposite sides of the leaflet 204 and a cusp edge portion 252 extending between the opposing commissure tabs. As described further below, the cusp edge portion 252 of each leaflet 204 can have an inflow end 226 (or edge, disposed at a central portion of the cusp edge portion 252) that is arranged at an inflow end of the prosthetic heart valve (e.g., inflow end 212 of a frame 202 of the prosthetic heart valve 200).

The frame 202 can be made of any of various suitable plastically-expandable materials (e.g., stainless steel, etc.) or self-expanding materials (e.g., Nitinol). In some examples, the frame 202 comprises a plastically-expandable material, such as those described above with reference to the prosthetic heart valve 10 of FIG. 1.

The frame 202 can comprise a plurality of interconnected struts 206 which form multiple rows of open cells 208 between an outflow end 210 and an inflow end 212 of the frame 202. In some examples, as shown in FIG. 3, the frame 202 can comprise three rows of cells 208 with a first (e.g., upper in FIG. 3) row of cells 214, disposed at the outflow end 210, having cells 208 that are elongated in an axial direction (relative to a central longitudinal axis 216 of the frame 202), as compared to cells 208 in the remaining rows of cells. For example, the cells 208 of the first row of cells 214 can have a longer axial length, defined in a direction of a central longitudinal axis 216 of the frame 202, than cells 208 in the remaining rows of cells (e.g., cells in the row of cells at the inflow end 212).

In some examples, as shown in FIG. 3, each row of cells 208 comprises nine cells. Thus, in such examples, the frame 202 can be referred to as a nine-cell frame.

In other examples, the frame 202 can comprise more than three rows of cells (e.g., four or five) and/or more or less than nine cells per row. In some examples, the cells 208 in the first row of cells 214 may not be elongated compared to cells 208 in remaining rows of cells of the frame 202.

The interconnected struts 206 can include a plurality of angled struts 218, 234, 236, and 238 arranged in a plurality of rows of circumferentially extending rows of angled struts, with the rows being arrayed along the length of the frame between the outflow end 210 and the inflow end 212 of the frame 202. For example, the frame 202 can comprise a first row of angled struts 238 arranged end-to-end and extending circumferentially at the inflow end 212 of the frame; a second row of circumferentially extending, angled struts 236; a third row of circumferentially extending, angled struts 234; and a fourth row of circumferentially extending, angled struts 218 at the outflow end 210 of the frame 12. The fourth row of angled struts 218 can be connected to the third row of angled struts 234 by a plurality of axially extending window strut portions 240 and a plurality of axial (e.g., axially extending) struts 232. The axially extending window strut portions 240 define commissure windows (e.g., open windows) 242 that are spaced apart from one another around the frame 202, in a circumferential direction, and which are adapted to receive a pair of commissure tabs of a pair of adjacent leaflets 204 arranged into a commissure 230. The window strut portions 240 forming the commissure windows 242 can also be referred to herein as commissure supports of the frame 202.

One or more (e.g., two, as shown in FIG. 3) axial struts 232 can be positioned between, in the circumferential direction, two commissure windows 242 formed by the window strut portions 240. Since the frame 202 can include fewer cells per row (e.g., nine) and fewer axial struts 232 between each commissure window 242, as compared to other prosthetic heart valves, such as the prosthetic heart valve 10 of FIG. 1, each cell 208 can have an increased width (in the circumferential direction), thereby providing a larger opening for blood flow and/or coronary access, as described herein.

Each axial strut 232 and each window strut portion 240 extends from a location defined by the convergence of the lower ends (e.g., ends arranged inward of and farthest away from the outflow end 210) of two angled struts 218 (which can also be referred to as an upper strut junction or upper elongated strut junction) to another location defined by the convergence of the upper ends (e.g., ends arranged closer to the outflow end 210) of two angled struts 234 (which can also be referred to as a lower strut junction or lower elongate strut junction). Each axial strut 232 and each window strut portion 240 forms an axial side of two adjacent cells of the first row of cells 214.

In some examples, as shown in FIG. 3, each axial strut 232 can have a width 244 that is larger than a width of the angled struts 218, 234, 236, and/or 238. As used herein, a “width” of a strut is measured between opposing locations on opposing surfaces of a strut that extend between the radially facing inner and outer surfaces of the strut (relative to the central longitudinal axis 216 of the frame 202). A “thickness” of a strut is measured between opposing locations on the radially facing inner and outer surfaces of a strut and is perpendicular to the width of the strut. In some examples, the width 244 of the axial struts 232 is 50-200%, 75-150%, or at least 100% larger than (e.g., double) the width of the angled struts of the frame 202. For example, if the angled struts 218, 234, 236, and 238 are approximately 0.3 mm wide, then the width 244 of the axial struts 232 can be in a range of 0.45 mm-0.9 mm, 0.5 mm-0.75 mm, or at least 0.6 mm.

For example, in known prosthetic heart valves (such as the valve 10 shown in FIG. 1) the axial struts have a same width as the other struts (e.g., angled struts) of the frame. As such, when the leaflets of the known prosthetic heart valve are pressed against the frame during the systolic phase, the leaflets may bend around the struts and/or extend radially outwardly through the cell openings due to the relatively narrow width of the axial struts. This phenomenon can reduce long-term durability of the leaflets, especially when the upper (e.g., outflow/free) edges of the leaflets are pushing against the axial struts and are bent over, as described above.

Thus, by providing the axial struts 232 with the width 244 that is greater than the width of other struts (e.g., angled struts of the frame 202), a larger contact area is provided for when the leaflets 204 contact the wider axial struts 232 during systole. This can, for example, distribute the stress and reduce the extent to which the leaflets 204 fold over the axial struts 232 and/or extend radially outward through the cells 208. As a result, one advantage of the disclosed technology is that the long-term durability of the leaflets 204 is increased.

In some cases, the free edges at the outflow end 228 of the leaflets 204 may press against the axial struts 232 at their outflow (e.g., upper) end portions 246. Accordingly, in some examples, the outflow end portions 246 of the axial struts 232 can be even wider than the width 244, which is depicted at an intermediate location of the axial strut 232 in FIG. 3, thereby providing an even larger area of contact and support for the leaflets 204.

As introduced above, since the frame 202 can have fewer cells in the circumferential direction (e.g., nine in the example depicted in FIG. 3) compared to the prosthetic valve of FIG. 1, each cell 208 can have an increased width (measured in the circumferential direction). This increased width of the cells 208 of the first row of cells 214 can enable the wider axial struts 232 to be incorporated into the frame 202, without sacrificing open space for blood flow and/or coronary access.

Commissure tabs 250 of adjacent leaflets 204 can be secured together to form commissures 230. Each commissure 230 of the prosthetic heart valve 200 comprises two commissure tabs 250 paired together, one from each of two adjacent leaflets 204, and extending through a commissure window 242 of the frame 202. Each commissure 230 can be secured to the window strut portions 240 forming the commissure window 242.

The cusp edge portion (e.g., scallop edge) 252 of each leaflet 204 can be secured to the frame via one or more fasteners (e.g., sutures). In some examples, as shown in FIG. 3, the cusp edge portion of each leaflet 204 can be secured directly to the struts of the frame 202 (e.g., angled struts 234, 236, and 238). For example, the cusp edge portions 252 of the leaflets 204 can be sutured to the angled struts 234, 236, 238 that generally follow the contour of the cusp edge portions of the leaflets.

In some examples, the cusp edge portion 252 of the leaflets 204 can be secured to an inner skirt and the inner skirt can then be secured directly to the frame 202.

Further, in some examples, an outer skirt can be connected to an outer surface of the frame 202 (e.g., similar to the outer skirt 18 of the valve 10 of FIG. 1).

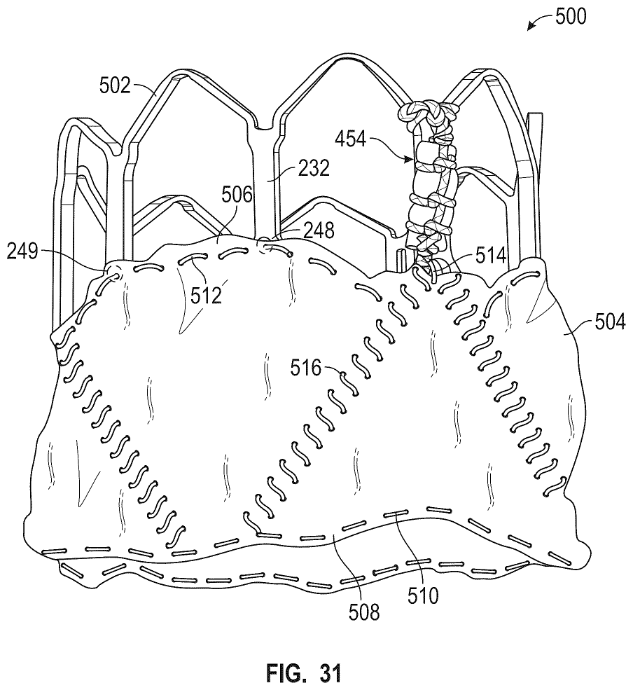

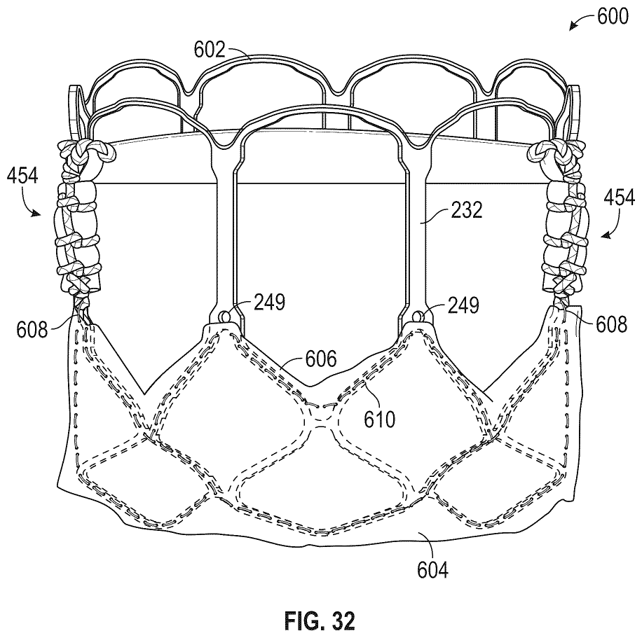

As shown in FIG. 3, in some examples, one or more of or each of the axial struts 232 can comprise an inflow end portion (e.g., inflow end portion that is closer to the inflow end than the outflow end portion 246) 248 that is widened relative to a middle portion 247 of the axial strut 232 (which can be defined by the width 244), similar to the outflow end portion 246 (as described above). In some examples, the inflow end portion 248 of the axial strut 232 can comprise an aperture 249. The apertures 249 can be configured to receive fasteners (e.g., sutures) for attaching soft components of the prosthetic heart valve 200 to the frame 202. For example, in some examples, an outer skirt can be positioned around an outer surface of the frame 202 and secured to the apertures 249 (e.g., as shown in FIGS. 31 and 32 and described further below).

The frame 202 can further comprise a plurality of apices 220 formed at the inflow end 212 and the outflow end 210, each apex 220 forming a junction between two angled struts 218 at the inflow end 212 or outflow end 210. As such, the apices 220 are spaced apart from one another, in a circumferential direction at the inflow end 212 and the outflow end 210.

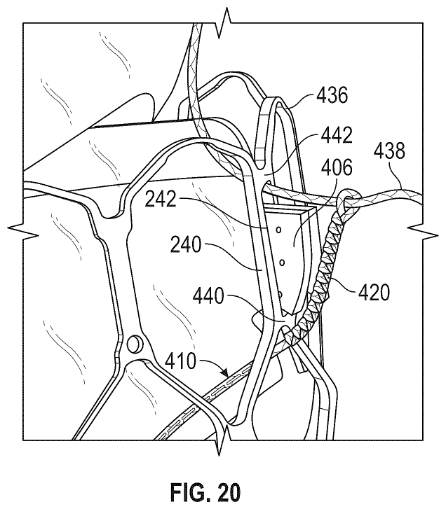

In some examples, as shown in FIG. 3, each apex 220 can have side portions 222 that curve or bend axially outward from the angled strut 218 to which it is connected and an end portion 224 that extends between the two side portions 222 of the apex 220. The side portions 222 can extend in a direction that is parallel to the central longitudinal axis 216. The end portion 224 can be relatively flat and include a surface that is disposed normal to the central longitudinal axis 216. Each apex 220 can have two bends at its end portion 224 and two bends at the side portions 222 (e.g., one at the junction between each side portion 222 and angled strut 218). In this way, the apices 220 can be U-shaped, similar to the apices 26 of the valve of FIG. 1. However, in some examples, the apices 220 can have another shape such as a more curved and longer apex region with a reduced height in the axial direction (e.g., as shown in FIGS. 20-32), such as disclosed in U.S. Provisional Application Nos. 63/178,416 and 63/194,830, both of which are incorporated herein by reference.

As introduced above, a leaflet assembly of a prosthetic heart valve comprising a plurality of leaflets (e.g., leaflets 204 shown in FIG. 3) can either be secured to an inner skirt which is then secured to the struts of the frame of the prosthetic heart valve or directly to the struts of the frame (without the inner skirt). An example of directly securing the leaflets 204 to the struts 206 of the frame 202 is shown in FIG. 3. Reducing the number of soft components of the prosthetic heart valve (e.g., by eliminating the inner skirt and suturing the leaflets directly to the frame) can simplify a process for assembling the prosthetic heart valve. For example, assembling prosthetic heart valves including an inner skirt can result in a prolonged assembly time which involves suturing each of the leaflets to the inner skirt and then suturing the inner skirt to the frame of the prosthetic heart valve.

Further, it can be difficult and/or time consuming to suture the leaflets to the frame. Thus, it may be desirable to assemble the leaflets into a leaflet assembly off the frame with a simplified sewing process, such that the leaflet assembly can be more easily and quickly secured to the frame.

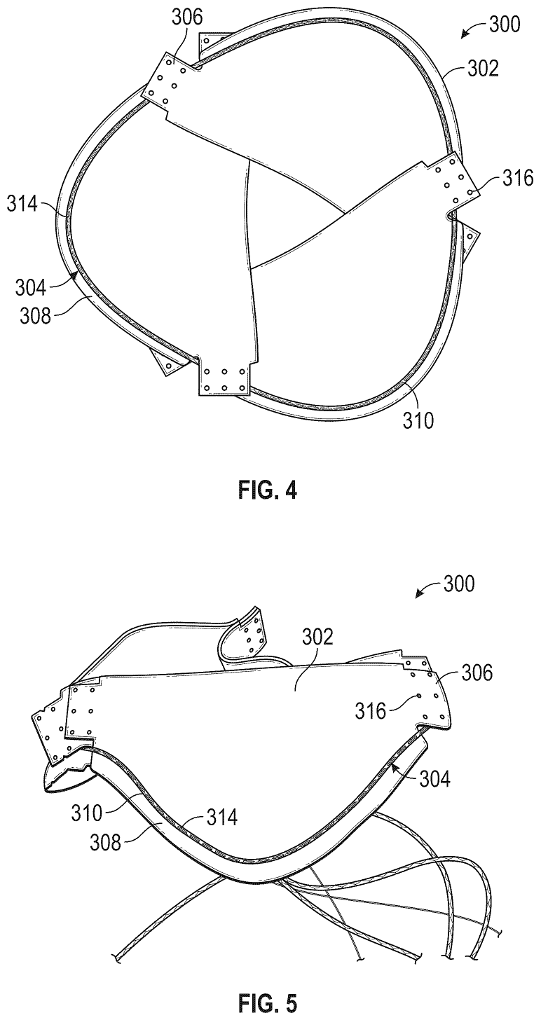

FIGS. 4-6 show leaflets 302 secured together into an exemplary leaflet assembly 300 (or sub-assembly) with a connecting suture line 304 (also referred to herein as “connectors”) disposed along a cusp edge portion of each of the leaflets 302. As described further below, the connecting suture line 304 is configured to enable securing of the leaflet assembly 300 directly to a frame of a prosthetic heart valve (e.g., the frame 202 of FIG. 3, as shown in FIGS. 7-13), without having to extend or thread sutures through the leaflet during assembly to the frame.

As used herein, a “connecting suture line” can be defined as a line of multiple sutures that extends along the cusp edge portion on at least one side of at least one leaflet and that comprises at least one first suture extending along the cusp edge portion on the at least one side of the at least one leaflet and a plurality of in-and-out stitches (e.g., formed with a second suture) that extend through the at least one first suture and the cusp edge portion of the leaflet. The in-and-out stitches can secure the first suture to the leaflet. The connecting suture line can be formed on the leaflet while the leaflet is off the frame. Further, the connecting suture line can be formed (e.g., stitched) manually or automatically.

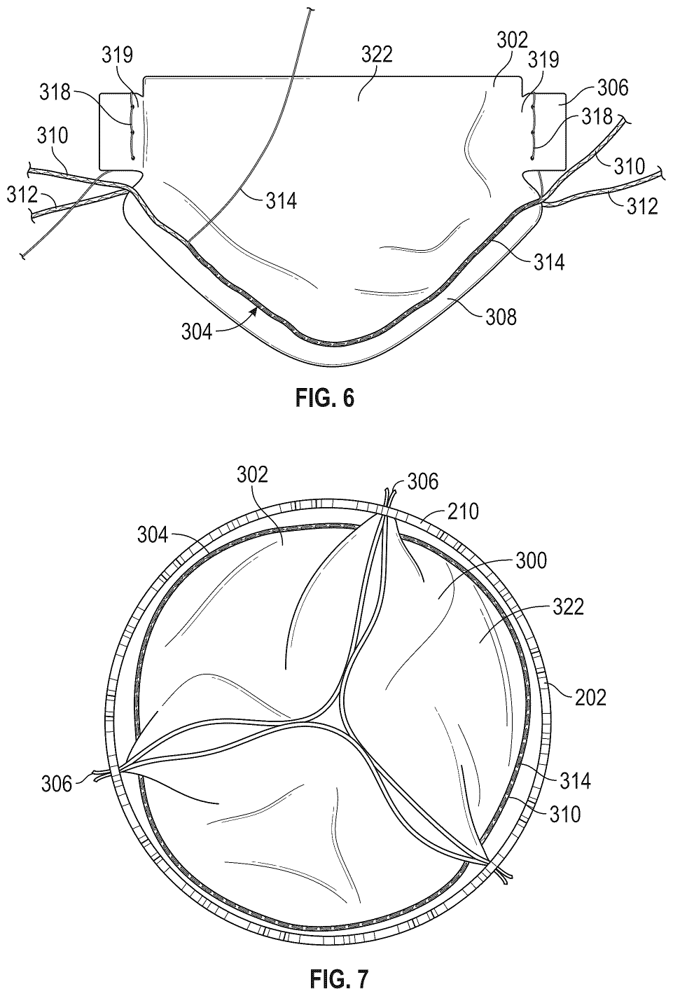

Returning to FIGS. 4-6, each leaflet 302 can comprise opposing commissure tabs 306 disposed on opposite sides of the leaflet 302 and a cusp edge portion 308 extending between the opposing commissure tabs 306 (FIGS. 4-6). FIG. 6 shows a single leaflet 302 while FIGS. 4 and 5 show a flat view and perspective view, respectively, of the leaflet assembly 300 comprising three leaflets 302 secured together along their cusp edge portions 308 by the connecting suture line 304.

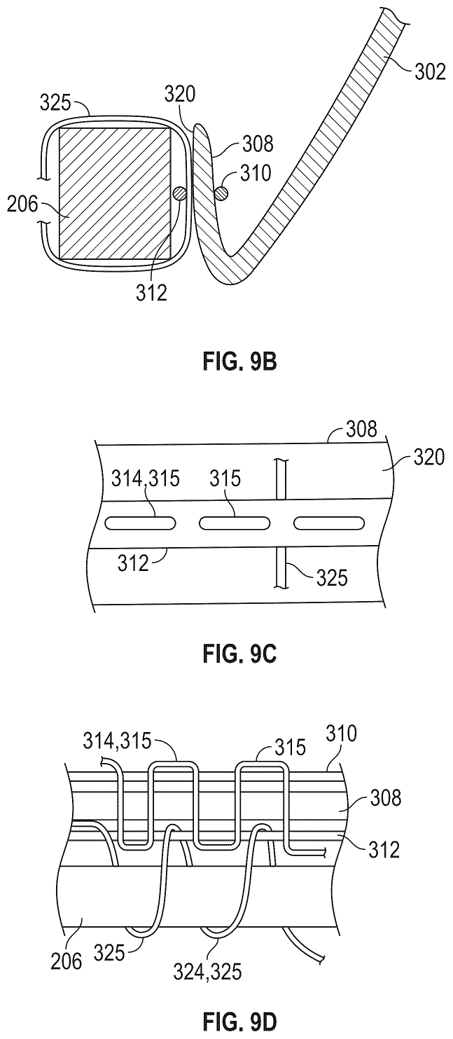

To form the connecting suture line 304, one or more first sutures (referred to herein as a first thicker suture or a first main suture) 310 can be paced along the cusp edge portions 308, on a first side of the leaflets 302 (FIGS. 4-6). In some examples, one or more second sutures (referred to herein as a second thicker suture or a second main suture) 312 can be placed along the cusp edge portions 308, on a second side of the leaflets 302, the second side arranged opposite the first side (FIG. 6 and also shown in FIG. 9B). In some examples, the same first thicker suture 310 and second thicker suture 312 can extend along the cusp edge portions 308 of each and every one of the leaflets 302 (e.g., three shown in FIG. 4) of the leaflet assembly 300, thereby connecting the leaflets 302 together. In other words, a single first thicker suture 310 can extend continuously along the cusp edge portions of all leaflets 302 on one side of the leaflets and a single second thicker suture can extend continuously along the cusp edge portions of all leaflets 302 on the opposite side of the leaflets.

In some examples, each leaflet 302 can include two thicker sutures 310 and 312 (or two thicker suture strands or portions of the same thicker suture) extending along its cusp edge portion 308, on each side of the leaflet 302, and the two thicker sutures 310 and 312 may or may not connect to the two thicker sutures 310 and 312 of the other leaflets 302 of the leaflet assembly 300.

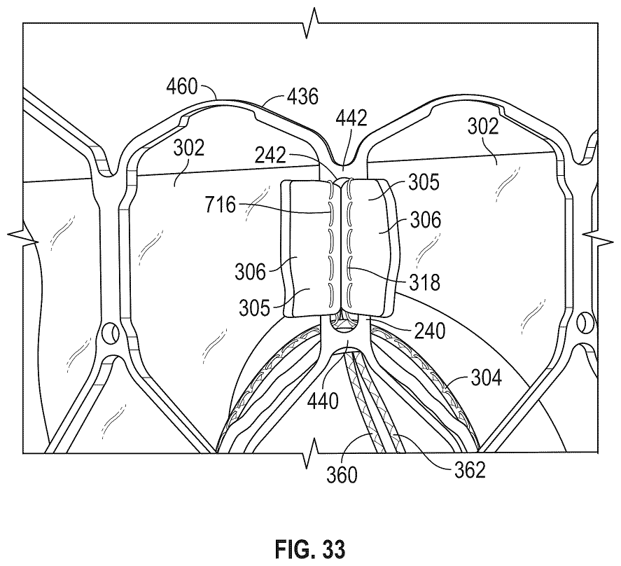

In some examples, the leaflet assembly 300 can be formed with one main or thicker suture (e.g., second suture 312) extending along a first side (e.g., the inner or rougher side 320) of the cusp edge portion 308 of all three leaflets 302 and three separate (or individual) first thicker sutures 310, each extending along a second side (e.g., the outer or smoother side 322) of the cusp edge portion 308 of a corresponding leaflet 302, thereby resulting in two suture end portions (also referred to as suture tails or suture segments) on either end of the cusp edge portion 308 of the leaflet that can extend beyond the commissure tabs 306. As a result, each pair of adjacent suture end portions (or tails) from adjacent leaflets 302 can be joined to form an integral wedge member during commissure assembly (as described further below with reference to FIGS. 33-43 and FIGS. 44-59).

To further form the connecting suture line 304 and connect the first thicker suture(s) 310 and/or the second thicker suture(s) 312 to the leaflets 302, a stitching suture (or third suture) 314 is stitched in a running-stitch pattern (e.g., an in-and-out pattern) through the first thicker suture 310 and/or the second thicker suture 312 and the leaflets 302 (e.g., through the thickness of the tissue of the leaflets 302). For example, if a leaflet 302 includes the first thicker suture (or suture strand or portion) 310 on its first side and the second thicker suture (or suture strand or portion) 312 on its second side, the stitching suture 314 can secure both the first thicker suture 310 and the second thicker suture 312 to the cusp edge portion 308 of the leaflet 302. The stitching suture 314 can form a plurality of in-and-out stitches 315 (as best shown in FIG. 9C), each of which can extend through the first suture 310, the cusp edge portion 308 of a leaflet 302, and the second suture 312 (as shown in FIG. 9D).

In certain examples, the stitching suture 314 is thinner than the suture(s) 310 and/or the suture(s) 312 to facilitate stitching of the stitching suture 314 through sutures 310, 312. Thus, the stitching suture 314 can be referred to as “the thinner suture” in the description below. In such examples, the stitching suture 314 has a smaller diameter and higher gauge than sutures 310, 312. However, it should be noted that in some examples, the stitching suture 314 need not be thinner than sutures 310, 312. In such examples, the stitching suture 314 can have the same diameter (and gauge) as the sutures 310, 312 or a greater diameter (and lower gauge) than sutures 310, 312.

FIG. 6 shows a view of one leaflet 302 with the thinner suture 314 being stitched in the running-stitch pattern through the first thicker suture 310, the cusp edge portion 308 of the leaflet 302, and the second thicker suture 312 (positioned behind the leaflet 302 in the view of FIG. 6), thereby coupling the first thicker suture 310 and the second thicker suture 312 to the leaflet 302 and creating the connecting suture line 304.

In some examples, the commissure tabs 306 of the leaflets 302 can comprise one or more apertures 316 through the tissue of the commissure tabs 306 (FIGS. 4 and 5). The apertures 316 can be configured to receive one or more fasteners (e.g., sutures) for fastening the commissure tabs to other components of the prosthetic heart valve (e.g., the frame, other commissure tabs, wedge members, or the like).

In some examples, another stitching suture (also referred to as another thinner suture) (which can be separate from or part of the thinner suture 314) can extend through the commissure tabs 306 (through the leaflet tissue or through apertures 316) to form a stitch line 318 (FIG. 6). This stitch line 318 can be used to attach the commissure tabs 306 to other components, such as a wedge or wedge member during attaching the leaflet assembly 300 to the frame of the prosthetic heart valve, as described further below.

Thus, the leaflet assembly 300, which includes the leaflets 302 (e.g., three leaflets) assembled together with the connecting suture line 304, can be formed off the frame of the prosthetic heart valve, in a flat configuration (as shown in FIG. 4). By assembling the leaflet assembly 300 off the frame in a flat configuration, the leaflet assembly 300 can be formed more easily and quickly. For example, this can allow the connecting suture line 304 to be sewn automatically (e.g., with sewing machinery) across the flat leaflets 302. Advantageously, this can significantly reduce manufacturing time and costs.

After forming the leaflet assembly 300, the leaflet assembly 300 can be attached to an inner surface of the frame of the prosthetic heart valve (e.g., frame 202 shown in FIGS. 7-9). As shown in FIGS. 7-9D, attachment of the leaflets 302 of the leaflet assembly 300 to the frame 202 can involve folding the cusp edge portions 308 of leaflets 302 such that an inner side 320 (which can be a rougher side of the tissue, and thus referred to as a rough side of the leaflets 302) of the cusp edge portions 308 of the leaflets 302 are facing the frame 202 (FIGS. 9A and 9B).

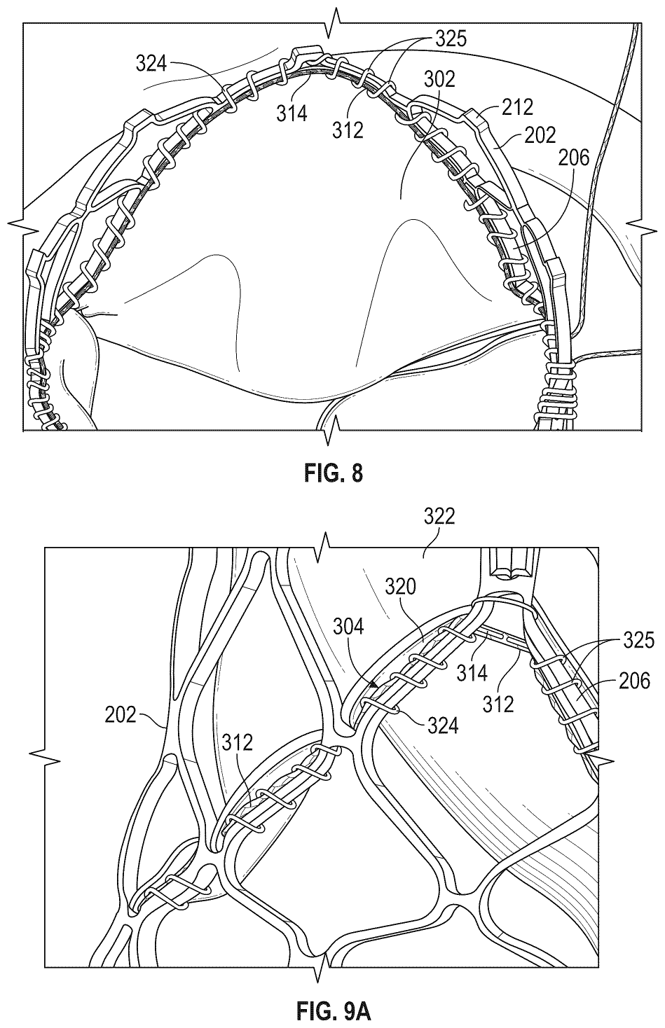

An outer side 322 of the leaflets 302 (the side of the leaflets 302 opposite the inner side 320, which can be a smoother side of the leaflets) is shown in FIGS. 6 and 7. FIG. 7 shows a view of the frame 202 from the outflow end 210 and FIG. 8 shows a view of the frame 202 from the inflow end 212. FIG. 7 also shows pairs of commissure tabs 306 of the leaflets 302 extending radially through commissure windows of the frame 202 that are formed by commissure supports of the frame 202 (e.g., window strut portions 240 shown in FIG. 3).

A connecting suture 324, such as a force fiber or UHMPE suture, can be looped around the struts 206 of the frame 202 and a portion of the connecting suture line 304 in order to attach the leaflet assembly 300 to the frame 202 without having to thread a needle and the connecting suture 324 through the tissue of the leaflets 302 (FIGS. 8 and 9A), thereby avoiding puncturing the leaflets or sewing the suture 324 through to the leaflets while assembling the leaflets to the frame.

For example, in some examples, the connecting suture 324 can be looped around the struts 206 and through the portions of the connecting suture line 304 between the second thicker suture 312 and the leaflets 302 (e.g., between the leaflets and the segments of the second thicker suture 312 that extend between the crossing points/stitches 315 formed by the thinner suture 314) (FIGS. 8 and 9A-9D). In this manner, the connecting suture 324 forms a plurality of whip stitches 325, each of which extends around a frame strut 206 and around the suture 312 without extending through the leaflet (FIGS. 9B and 9D). In some examples, one or more of the whip stitches 325 can extend around the suture 312 at locations underneath the stitches 315 instead of at locations between adjacent stitches 315. The connecting suture 324 can be used to secure the cusp edge portions 308 of the leaflets to the frame struts that generally track the curvature of the cusp edge portions. In particular, the connecting suture 324 can form whip stitches 325 along struts 234, 236, and 238 that generally track the curvature of the cusp edge portions 308.

In some examples, the connecting suture 324 can be routed through the second thicker suture 312, or between the stitches 315 and the second thicker suture 312.

In some examples, since the connecting suture 324 need not extend through any material, a blunt needle can be used for passing the connecting suture 324 through/around the connecting suture line 304.

The direct attachment of the leaflet assembly 300 to the frame 202, as shown in FIGS. 7-9 can allow for the elimination of an inner skirt in the assembled prosthetic heart valve. The connecting suture line 304 allows for the direct attachment of the leaflet assembly 300 to the frame 202, without puncturing the tissue of the leaflets 302 during assembly of the leaflet assembly 300 to the frame 202. Further, by utilizing thicker sutures 310 and/or 312 that have a greater thickness that other sutures, such as the thinner suture 314, the thinner suture 314 can be prevented from tearing through a tissue of the leaflets 302, thereby creating a more robust connection between the leaflet assembly 300 and the frame 202.

The prosthetic heart valve can also comprise an outer skirt 326 that is disposed around and attached to an outer surface of the frame 202 (such as the outer skirt 18 shown in FIG. 1, the outer skirt 504 shown in FIG. 31, or the outer skirt 604 shown in FIG. 32).

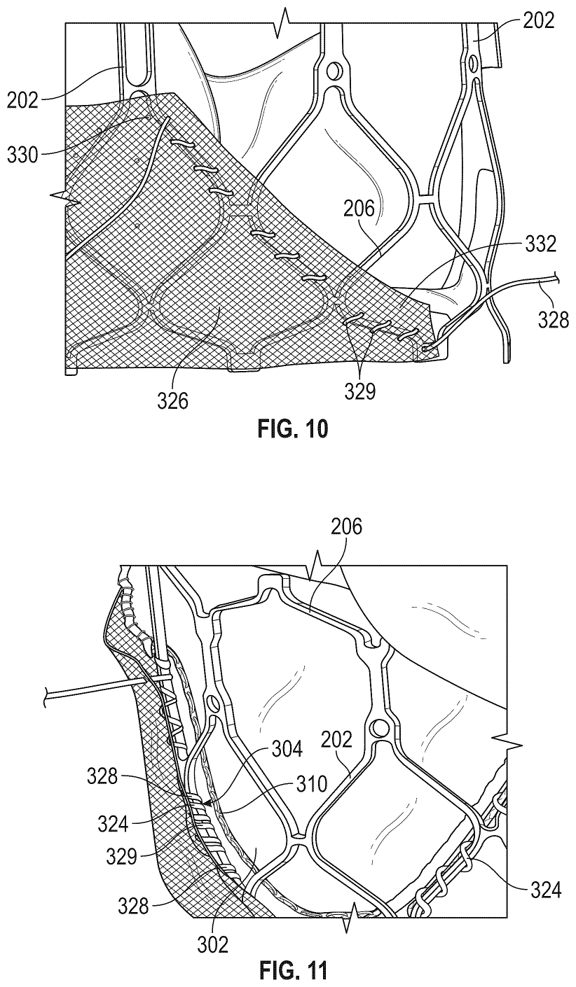

In some examples, as shown in FIGS. 10-12, the outer skirt 326 can be provided in the form of three separate skirt portions 332, each attached by passing a suture 328 through the fabric of the outer skirt 326, around a frame strut 206, and optionally through/around one of the thicker sutures 310 or 312 of the connecting suture line 304 of the leaflet assembly 300. For example, the suture 328 can form a plurality of whip stitches 329, each of which extends around a frame strut 206, through the skirt portion 332 and optionally around the suture 312 (e.g., each stitch 329 can extend between the suture 312 and a cusp edge portion 308). In some examples, the outer skirt 326 (or skirt portions 332) can include one or more preformed apertures 330 configured to receive the suture 328. FIG. 10 shows an outer surface of the frame 202 and the outer skirt 326, showing one of the skirt portions 332 of the outer skirt 326 being attached to the frame 202. As shown in the perspective view of FIG. 11, the suture 328 can secure the outer skirt 326 to the connecting suture line 304 on the leaflet 302 and to the frame 202 and the connecting suture 324 can secure the leaflets 302 to the struts 206 of the frame 202.

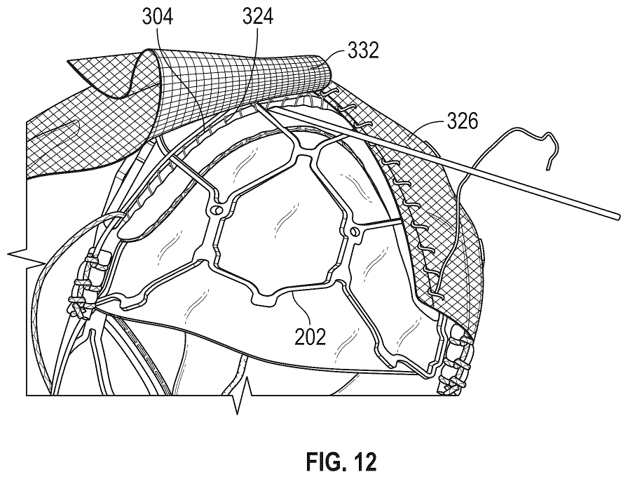

As shown in FIG. 12, a portion of the skirt portion 332 of the outer skirt 326 can be folded over itself during the assembly process to expose the portion of the cusp edge portions (e.g., scallop line) of the leaflets 302 (and the connecting suture line 304) to which it should be sutured. The ability to fold the separate skirt portions 332 of the outer skirt 326 over, as shown in FIG. 12, can simplify the suturing and assembly process.

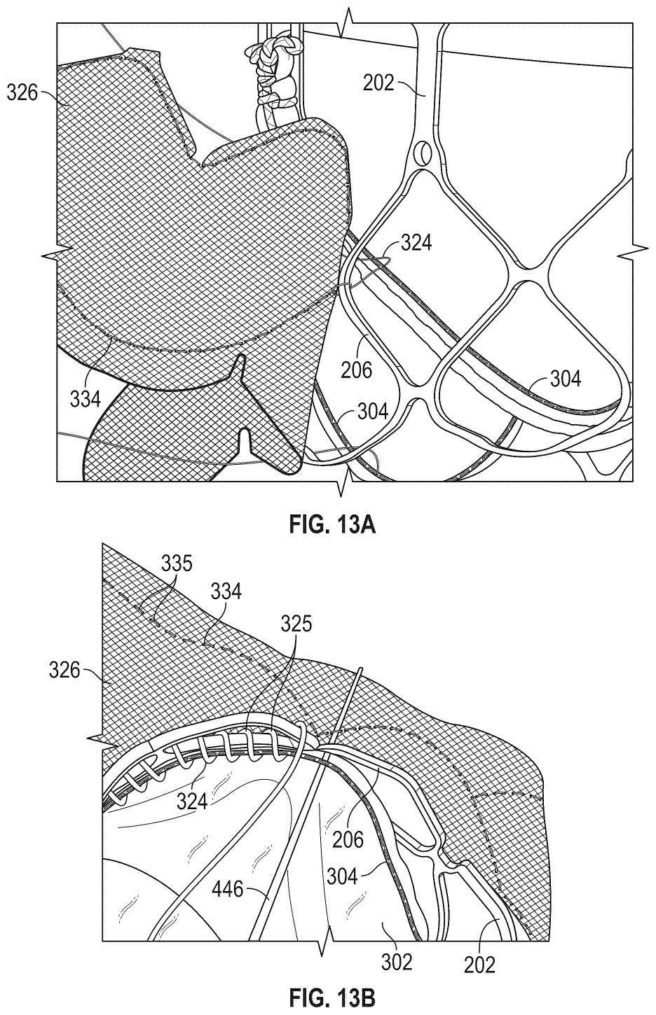

In some examples, as shown in FIGS. 13A and 13B, the outer skirt 326 (which may include one or more skirt portions 332, as described above) can include a connecting suture line 334 configured as running stitch (e.g., in-and-out stitch) forming a plurality of stitches 335 along an edge of the outer skirt that follows the cusp edge portions 308 of the leaflets 302 when attached to the frame 202. For example, the connecting suture line 334 can be stitched onto the outer skirt 326 off the frame 202 and then the outer skirt 326 and the leaflet assembly 300 can be attached to the frame 202 at the same time using the same (or a common) connecting suture 324.

In some examples, the connecting suture 324 can connect to the connecting suture line 304 of the leaflet assembly, as described above with reference to FIGS. 7-9, and then also connect to the connecting suture line 334 of the outer skirt 326 and around the struts 206, thereby securing the outer skirt 326 to the outer surface of the frame 202 and the leaflet assembly 300 to the inner surface of the frame 202 (FIG. 13A). For example, FIG. 13B shows an exemplary example of a needle 446 (or other needle) being threaded underneath the connecting suture line 304 of the leaflet, underneath the strut 206 of the frame 202, and underneath one of the stitches 335 of the connecting suture line 334 of the outer skirt 326, with the needle being connected to the connecting suture 324. Each stitch 325 therefore extends around a stitch 335 on the skirt. As a result, the outer skirt 326 and the leaflet assembly 300 can be simultaneously connected to the struts of the frame 202 with a common connecting suture 324 (or multiple common connecting sutures), using the respective connecting suture lines 334 and 304 of the outer skirt and leaflets of the leaflet assembly 300. By utilizing the connecting suture line 304 and the connecting suture line 334, the outer skirt 326 and leaflet assembly 300 can be more easily secured to the frame, without having to puncture the tissue of the leaflets 302 and fabric of the outer skirt 326.

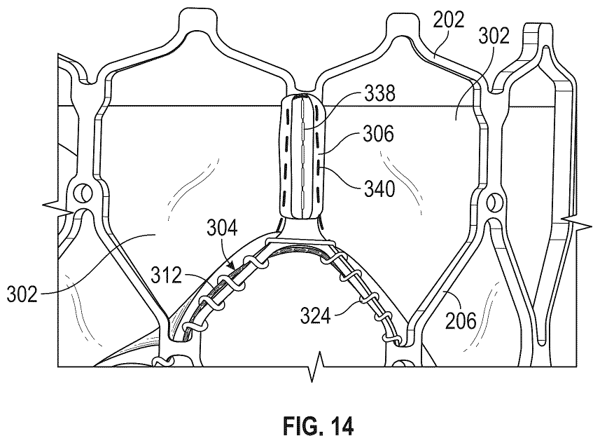

In some examples, one or more of the first thicker suture 310 (or multiple first thicker sutures 310 from adjacent leaflets) and the second thicker suture 312 can be further utilized as (or to form) a commissure wedge member. FIG. 14 shows a conventional configuration for a commissure which includes a wedge member 338 arranged between the commissure tabs 306 of two adjacent leaflets 302 that extend through a commissure window 242 of the frame 202. The wedge member 338 is configured prevent the commissure tabs 306 from pulling back through the commissure window 242 (e.g., into an interior of the frame 202), thereby maintaining the commissure tabs 306 secured to the commissure window 242.

FIG. 14 also shows a new wedge member 340 in addition to the conventional wedge member 338 for purpose of comparison. Instead of utilizing a separate wedge member 338 for the commissure, one or more of the first thicker suture(s) 310 and the second thicker suture 312 can extend from the cusp edge portions 308 of the leaflets 302, further upward along the commissure tabs 306 to form the wedge member 340 (shown schematically in FIG. 14 with dashed lines). For example, the second thicker suture 312 can continue from the cusp edge portion 308 of a first leaflet 302 and extend upward along the commissure tab 306 of the first leaflet and back around and over the commissure tab 306 of a second leaflet (which is adjacent to the first leaflet), such that once a commissure is formed, both portions of the second thicker suture 312 are juxtaposed to each other between both the commissure tabs 306 of the commissure, together forming a wedge member 340 that is thick enough to hold the commissure in place and prevent the commissure tabs 306 from being pulled back through the commissure window 242. Thus, a separate thick wedge member, such as the wedge member 338, is not needed.

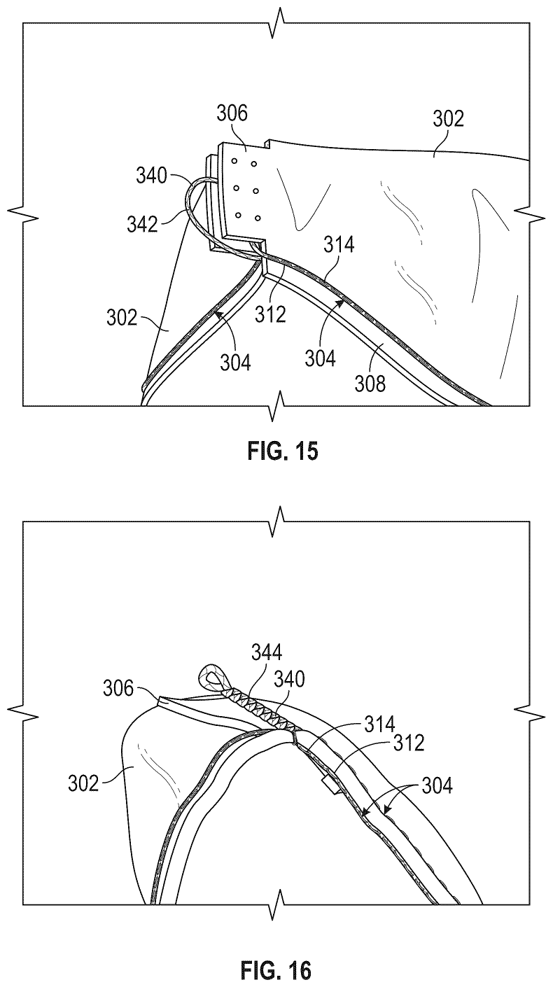

In one example, as shown in FIG. 15, the wedge member 340 can be formed as a loop 342 (e.g., a loop-shaped portion of the second thicker suture 312). The ends of the loop 342 can extend from the cusp edge portions 308 of the two adjacent leaflets 302. The loop 342 can extend and be wedged between the commissure tabs 306 of the adjacent leaflets 302 and be secured to the commissure tabs 306 as described further herein. The wedge member 340 formed as the loop 342 thus comprises two thicker suture segments positioned side-by-side between two commissure tabs 306.

In some examples, as shown in FIG. 16, the wedge member 340 can be formed as a single wedge member 344 (e.g., single segment wedge member) of the second thicker suture 312 (or the first thicker suture 310). The wedge member 344 can be formed by extending the second thicker suture 312 from one cusp edge portion 308 of one leaflet 302, to a length extending the height of the commissure tabs 306, thereby forming a first portion of the wedge member 344. Then, instead of looping back downward, the second thicker suture 312 can be passed through the first portion (e.g., the suture is pulled through or threaded through itself), to form the single wedge member 344 (e.g., one integrated, thick wedge with a thickness of two suture widths due to the overlapping portions of the thicker suture).

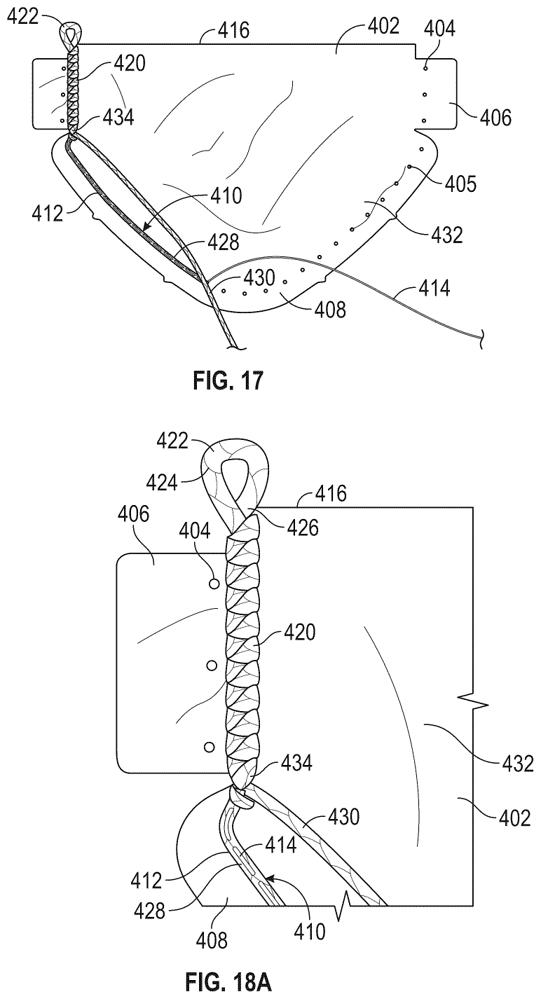

FIGS. 17-30 present an example for forming a wedge member 420 for a commissure of a prosthetic heart valve using one or more main sutures of a connecting suture line formed along the cusp edge portions of the leaflets. FIG. 17 shows a leaflet 402 (which can be the same or similar to the leaflet 302 of FIGS. 4-6) including opposing commissure tabs 406 and a cusp edge portion 408 extending between (e.g., in a scallop shape) the opposing commissure tabs 406.

The commissure tabs 406 can include one or more apertures 404 therein. Though three apertures 404 are shown in each commissure tab 406, in other examples each commissure tab 406 can include more or less than three apertures 404 (e.g., two, six, eight, or the like). In some examples, as shown in FIG. 17, apertures 405 can also be disposed in the cusp edge portion 408 of the leaflet 402. The apertures 404 and/or apertures 405 can be pre-formed in the leaflet 402 by one or more of punching (e.g., punching a hole into the leaflet and removing the punched-out tissue portions), laser heating, and/or by using a femtolaser to form the apertures. The apertures 404 and/or 405 are configured to receive the sutures described herein (such as the thinner suture 414 described below), thereby enabling a needle to pass a suture more easily through the tissue of the leaflet 402. For example, a blunt needle can be used to pass the sutures through the pre-formed apertures 404 and/or 405, since the pre-formed apertures can obviate the need to puncture the tissue of the leaflets 402 with a sharper needle. This can save manufacturing time and effort, as described further herein.

Similar to as described above with reference to FIGS. 4-6, a connecting suture line 410 is formed along the cusp edge portion 408 of the leaflet 402. The connecting suture line 410 includes one or more first sutures or suture segments that extend along the cusp edge portion 408, on one or both sides of the leaflet 402, and one or more second sutures 414 that is stitched in a running stitch (in-and-out) pattern through the first sutures or suture strands and the leaflet 402, as described above with reference to FIGS. 4-6. In some examples, the one or more first sutures can be thicker than the one or more second sutures 414, and thus, the first sutures can be referred to as thicker sutures and the second sutures 414 can be referred to as thinner sutures 414.

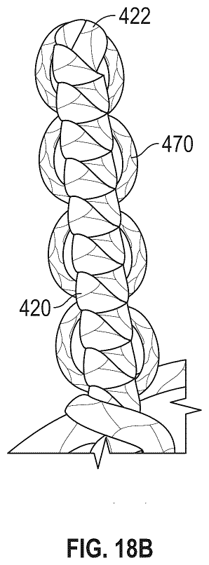

In some examples, a thicker (or main) suture 412 can be used to form both the wedge member 420 and the connecting suture line 410 (or at least one side of the connecting suture line 410). For example, as shown in FIGS. 17 and 18A, the wedge member 420 can be formed by extending a first portion 424 of the thicker suture 412 along a commissure tab 406 of the leaflet 402 and folding it over at a top of the commissure tab 406, thereby forming a loop 422, and then threading a second portion 426 of the thicker suture 412 through the first portion (e.g., through the braided filaments of the thicker suture 412). The resulting wedge member 420 is an integrated wedge member with a thickness of two suture widths (e.g., due to the overlapping first and second portions of the thicker suture 412).

In some examples, the thicker suture 412 can extend past (above in FIGS. 17 and 18A) a free, outflow edge 416 of the leaflet 402, thereby resulting in the loop 422 extending past the outflow edge 416 of the leaflet 402.

In some examples, the wedge member 420 can be formed by looping or folding the thicker suture 412 and/or an additional (thicker) suture one or more times to form a wedge member 420 with a thickness of more than two suture widths (e.g., three, four, or the like).

In some instances, an additional suture 470 that is a different color than the suture(s) forming the wedge member 420 can be passed (or threaded) through the wedge member 420 (or any of the other wedge members described herein) in order to further thicken and color-differentiate the wedge member 420. For example, as shown in the exemplary side view of the wedge member 420 in FIG. 18B, the suture 470 can be passed through the wedge member 420 in an in-and-out pattern along all or at least a portion of a length of the wedge member 420. In some examples, the suture 470 can be the thinner suture 414, which can have a different color than the sutures forming the wedge member 420. By using the same thinner suture 414 of the connecting suture line 410 for the suture 470, the same in-and-out suture line can be continued from the leaflets to the wedge member 420, thereby reducing components used in the assembly and simplifying the assembly process. Further, by color-differentiating the suture 470, the wedge member 420 can be more easily visualized by a user during assembly of the leaflet assembly and commissure.

Returning to FIGS. 17 and 18A, two segments (end segments) of the thicker suture 412 (resulting from formation of the loop 422 with the thicker suture 412) can extend away from a bottom 434 of the wedge member 420 and can then be used to form the connecting suture line 410 along the leaflet 402 and/or additional leaflets of the leaflet assembly. For example, as shown in FIGS. 17 and 18A, a first thicker suture segment 428 and a second thicker suture segment 430 of the thicker suture 412 extend away from the bottom 434 of the wedge member 420 and along the cusp edge portion 408 of the leaflet 402. Free ends of the first thicker suture segment and the second thicker suture segment 430 can then be buried or knotted along the cusp edge portion 408 of the leaflet 402. This can be repeated for each wedge member and leaflet of the leaflet assembly (e.g., each of three wedge members can have thicker suture segments that extend along one of the three leaflets of the leaflet assembly).

The first thicker suture segment 428 can extend over a first side 432 of the leaflet, along the cusp edge portion 408 and a form a portion of the connecting suture line 410 with the thinner suture 414 (FIGS. 17 and 18A).

In some examples, the second thicker suture segment 430 can wrap around to the opposite, second side of the leaflet 402 (facing into the page and not visible in FIGS. 17 and 18A since it's opposite (behind) the first side 432 which is in a plane of the page in FIGS. 17 and 18A) and then extend (and run) along the cusp edge portion 408 on the second side of the leaflet 402. In this way, the first thicker suture segment 428 and the second thicker suture segment 430 can run along the same cusp edge portion 408 of the same leaflet 402 (on opposite sides of the leaflet 402).

In some examples, the second thicker suture segment 430 can instead extend along the cusp edge portion 408 of a second, adjacent leaflet 402. In some examples, for the sake of symmetry, one of the first thicker suture segment 428 and the second thicker suture segment 430 can extend along a first side (e.g., a smooth side) of a first leaflet and the other one of the first thicker suture segment 428 and the second thicker suture segment 430 can extend along a second side (e.g., rough side) of an adjacent, second leaflet. This can then be repeated for each wedge member and set of adjacent leaflets of the leaflet assembly.

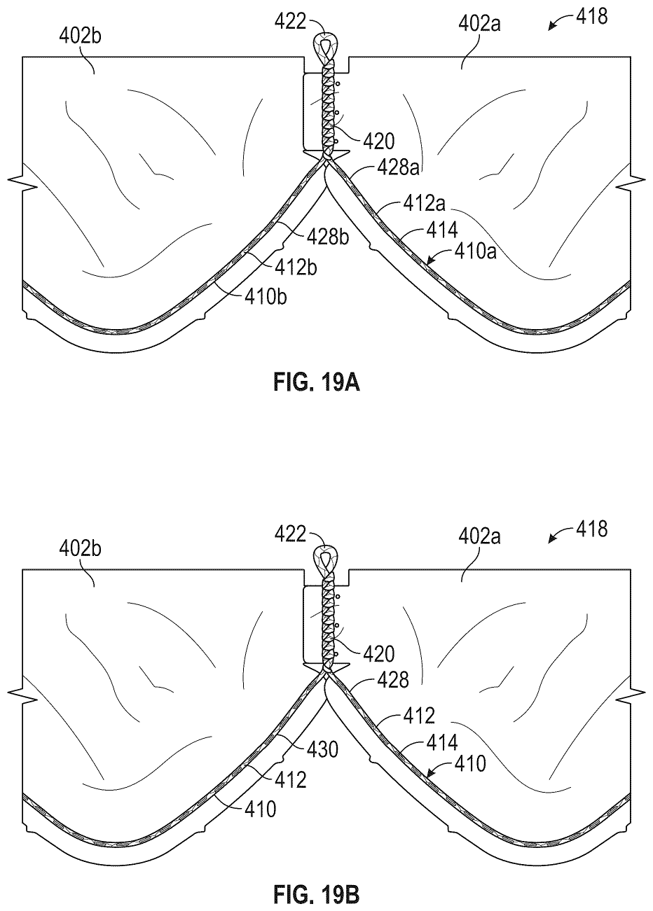

A plurality of leaflets 402 (e.g., three) can be assembled in a flattened (or flat) configuration, outside a frame of the prosthetic heart valve, as described above with reference to FIG. 4 and as shown in FIGS. 19A and 19B (only two leaflets 402 are shown in FIGS. 19A and 19B). As introduced above, such flattened configurations can enable utilization of automated or semi-automated techniques for suturing the leaflets outside the frame (e.g., connecting the leaflets into a leaflet assembly 418 and forming the connecting suture line 410).

For the purposes of discussion, FIGS. 19A and 19B are similar to FIG. 17 and show different examples of forming the connecting suture line 410 along a first leaflet 402a and second leaflet 402b. FIG. 19A shows one example where a first thicker suture 412a forms the wedge member 420 and the connecting suture line 410a on the front and back sides of the first leaflet 402a. Similarly, a second thicker suture 412b forms another wedge member (not seen in FIG. 19A, but is at the left hand side of leaflet 402b) and the connecting suture line 410b on the front and back sides of the second leaflet 402b. In some examples, a free end of the second thicker suture 412b can be tied off or inserted into the wedge member 420.

As shown in FIG. 19A, the wedge member 420 extends along the commissure tabs 406 of two adjacent leaflets 402a and 402b, and a first thicker suture segment 428a of suture 412a and a corresponding second thicker suture segment 430 (FIG. 17; not shown in FIG. 19A) of suture 412a extending from the wedge member 420 can form the connecting suture line 410a, with the thinner suture 414, along at least the first leaflet 402a (e.g., the first thicker suture segment 428a and the second thicker suture segment 430 extend along opposite sides of the cusp edge portion 408 of the first leaflet 402a). Thus, in FIG. 19A, a single suture 412a forms the wedge member 420 and first and second thicker suture segments on opposite sides of the leaflet 402a.

Alternatively, as shown in FIG. 19B, starting from the wedge member 420, a first thicker suture segment 428 can extend along a first side of the first leaflet 402a and a second thicker suture segment 430 can extend along a first side of a second leaflet 402b, thereby forming at least a portion of the connecting suture line 410 with the thinner suture 412 along the first leaflet 402a and the second leaflet 402b (e.g., on a front side of the two leaflets). The connecting suture line 410 can be further formed by two segments of an additional thicker suture which extend along the opposite (or back) sides of the two leaflets. In some examples, instead of extending along the first side of the second leaflet 402b, the second thicker suture segment 430 can extend along a second side (e.g., back side in FIG. 19B) of the second leaflet 402b (e.g., for symmetry, as previously discussed above).

Once the leaflets 402 are connected together into the leaflet assembly 418, off the frame, and a wedge member 420 is formed for each pair of adjacent leaflets 402 (FIGS. 19A and 19B), the leaflet assembly 418 can be connected to a frame 436 (which can be similar to the frame 202 of FIG. 3) of a prosthetic heart valve, as shown at FIGS. 20-30.

For example, after forming the leaflet assembly 418 and positioning the leaflet assembly 418 inside the frame 436, each pair of commissure tabs 406 (one pair shown in FIG. 20) of each pair of adjacent leaflets 402 is passed through a corresponding commissure window 242 of the frame (FIG. 20). Though only one pair of commissures tabs 406 and a corresponding wedge member 420 is shown in FIGS. 20-30, the method described below for forming the commissure and attaching it to the frame 436 can be repeated for each pair of commissure tabs 406 and corresponding wedge member 420 of the leaflet assembly 418.

As shown in FIG. 20, the wedge member 420 can be passed from an end of the cusp edge portion 408 of one of the leaflets 402 (e.g., where the cusp edge portions 408 of two adjacent leaflets 402 meet), below a lower (or bottom) end 440 of the commissure window 242, and outward from the frame 436 to pass along an outer side of the commissure window 242 and along the commissure tabs 406.

In some examples, as shown in FIG. 20, the commissure tabs 406 can be relatively short and not extend too far out of the commissure window 242, thereby allowing them to overlap each other without excessive material getting in the way.

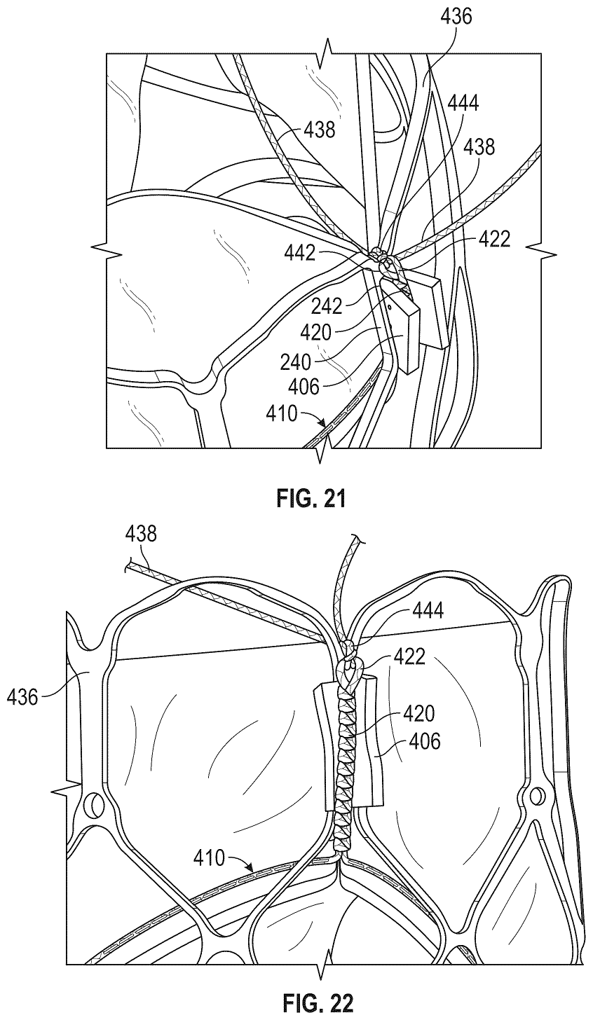

A suture 438 can then be used to tie the wedge member 420 to an upper end (or portion) 442 of the commissure window 242 (the upper end of the commissure support forming the commissure window) (FIG. 20) while the wedge member 420 is arranged between the adjacent commissure tabs 406, thereby forming an upper knot 444 and leaving both strands of the suture 438 extending from the upper knot 444 (FIGS. 21 and 22).

In some examples, the strut forming the upper end 442 of the commissure window 242 can include one or more apertures. In such examples, the suture 438 can also pass through the one or more apertures above the commissure window 242.

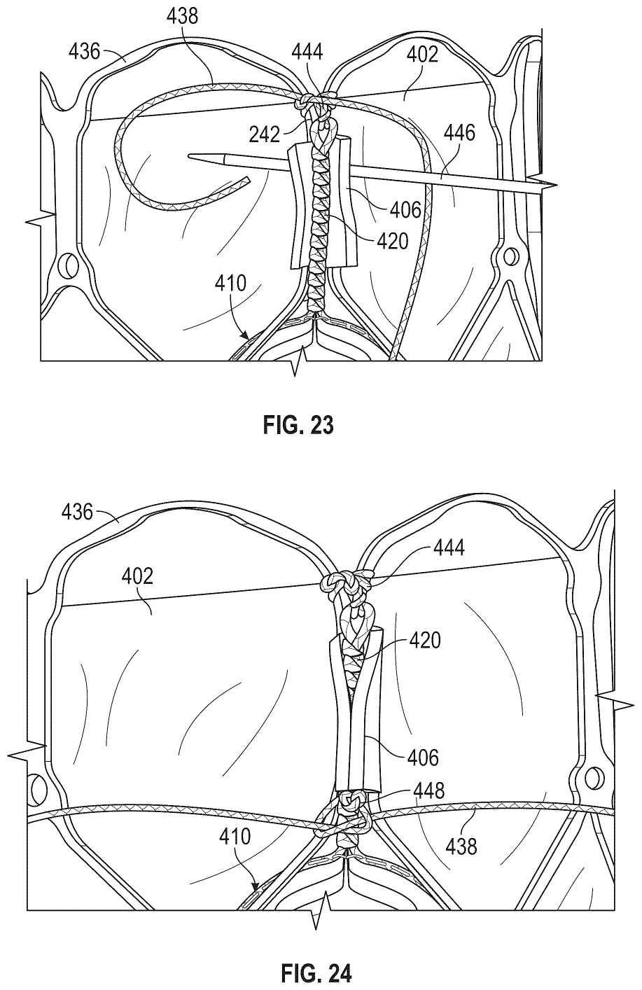

A needle 446 can then be used to pass the strands of the suture 438 in an in-and-out pattern downward from the upper knot 444 toward the lower end 440 of the commissure window 242 (the lower end of the commissure support forming the commissure window), with the strands of the suture 438 passing through the pre-formed apertures of both commissure tabs 406 and the wedge member 420 disposed therebetween (FIG. 23). In some examples, the needle 446 can be a blunt needle. This forms a first pass of stitches from the strands of suture 438 that secure the commissure tabs 406 to the wedge member 420.

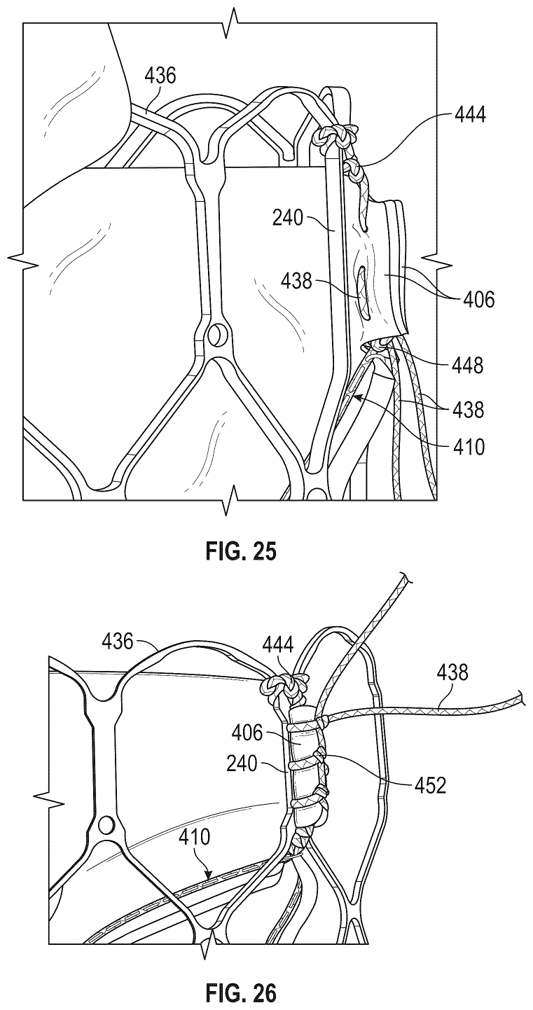

Both strands of the suture 438 can then be tied together below the commissure tabs 406, at the lower end 440 of the commissure window 242, thereby forming a lower knot 448 (FIGS. 24 and 25).

The (two) strands of the suture 438 can then be utilized to further stitch both commissure tabs 406 to each other by passing the strands of the suture 438 upward, toward the upper knot 444, and along the outer side of the commissure tabs 406 (FIG. 26). For example, as shown in FIG. 26, the strands of the suture 438 can pass through the commissure tabs 406 and form one or more looped knots 452 along and across the outer sides of the commissure tabs 406.

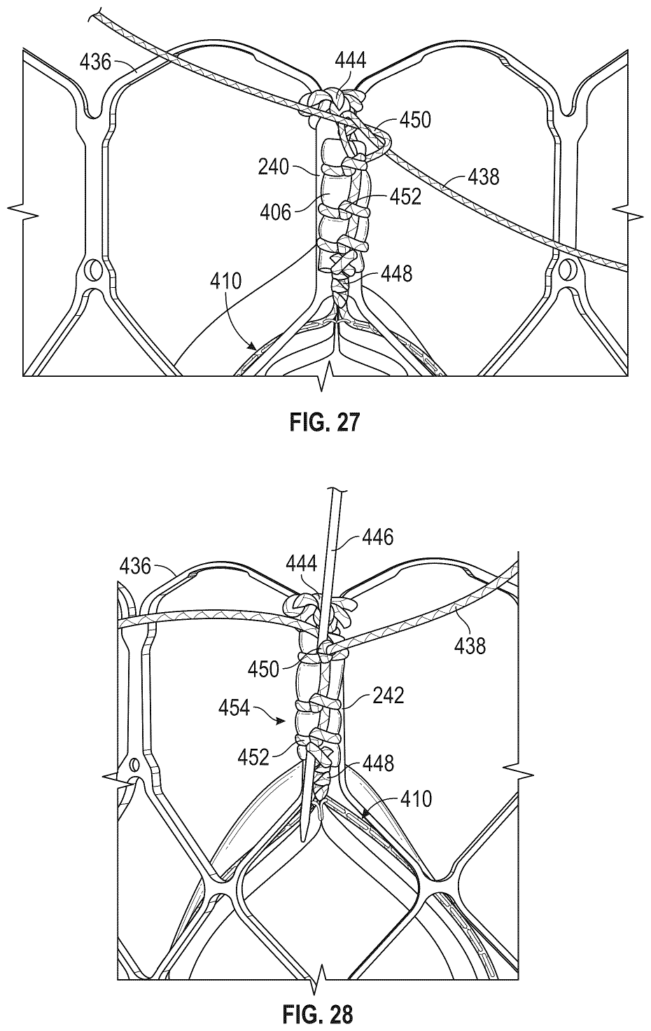

The strands of the suture 438, near an upper end of the commissure tabs 406 (and the upper end 442 of the commissure window 242), can then be tied to each other to form a final upper knot 450 above the commissure tabs (e.g., at an outflow end of the commissure tabs) (FIG. 27).

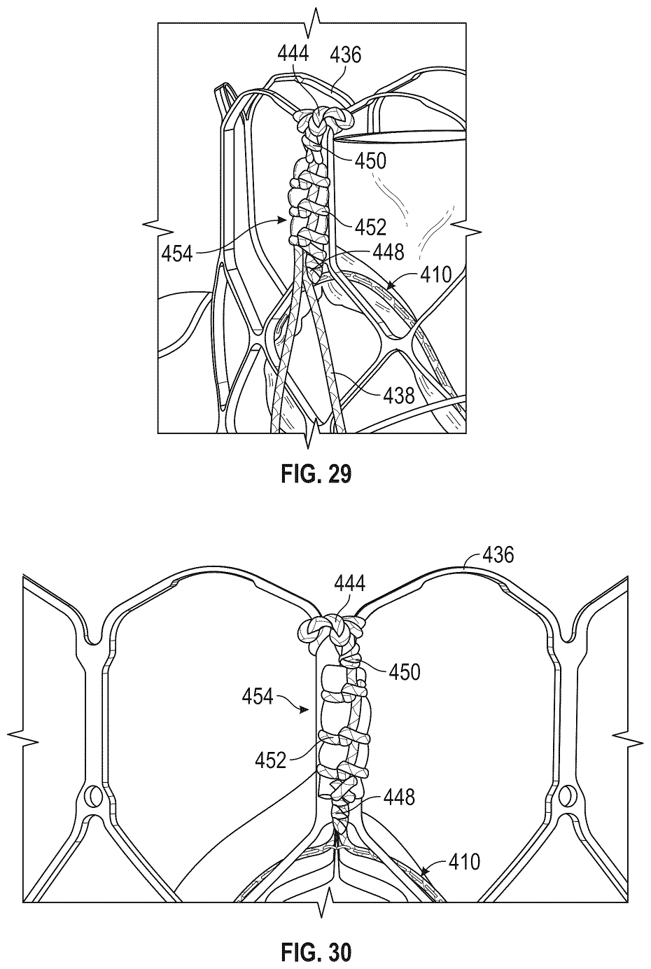

The needle 446 can be further utilized to pass both strands of the suture 438 from the final upper knot 450 downward, through the looped knots 452 across the commissure tabs 406 (FIG. 28). This can result in both strands of the suture 438 extending from below the formed commissure 454 (FIG. 29). In some examples, at this stage, the strands of the suture 438 can be cut to result in the final commissure 454 (FIG. 30).

In this way, leaflets of a leaflet assembly can be assembled together off the frame (e.g., in a flat configuration) and then can be directly coupled (e.g., sutured) to the struts of the frame, along the cusp edge portion (or scallop line) of the leaflets. Commissures of the leaflet assembly can be formed and attached to the commissure windows of the frame along a path that is directly continuous with the leaflet cusp edge portions or scallop line. As a result, stresses can be relatively uniformly distributed across the leaflets, without excess stresses developing at specific regions of the leaflets (such as between the commissure tabs and the cusp edge portions). As discussed above, the proposed commissure attachment configuration can simplify the assembly of the leaflet assembly to the frame, as well as reduce overall production time of the prosthetic heart valve.

As introduced above, a blunt needle can be utilized to form the commissures 454 (e.g., secure the commissure tabs 406 to the wedge member 420 for each commissure 454, as shown in FIGS. 20-30) by passing the blunt needle through apertures pre-formed in the commissure tabs (apertures 404 shown in FIGS. 17 and 18). The blunt needle can also be utilized in other phases of the assembly of leaflet assembly, such as passing the thinner suture 414 through the apertures 405 that can be pre-formed along the cusp edge portions of the leaflets 402, as described above with reference to FIG. 17.

Utilization of a blunt needle can offer several advantages over more conventional, sharp-pointed needles. One such advantage is increased durability since the thinner/sharper ends of more conventional needles can break more often than blunt needles, thereby saving production costs since a blunt needle may need replaced less often during the assembly process. Another advantage includes reduced risk of damage to a user (e.g., pricking a finger) and to the frame of the prosthetic heart valve (e.g., the sharp ends of more conventional needles may scratch or degrade a surface coating or treatment on the frame). Further, as discussed above, a blunt needle can more easily pass through pre-formed apertures in the leaflet without applying excessive force which can degrade the leaflets or other components of the prosthetic heart valve.

The various examples of forming the leaflet assembly and attaching the leaflet assembly to the frame, using the thicker and thinner sutures to form a connecting suture line and a commissure wedge member, as described above, can result in a simpler and more efficient valve assembly process. This can result in shorter prosthetic valve production times, which is advantageous for mass production scenarios.

In some examples, as shown in the example of FIG. 3, the axial struts 232 of the frame 202 of the prosthetic heart valve can include lower apertures 249 through which an outer skirt can be sutured to the frame 202, while the axially extending window strut portions 240 forming the commissure windows 242 may be devoid of such apertures. As a result, a portion of the outer skirt that extends across the lower regions of the axially extending window strut portions 240 can be loose and unattached to the frame 202.

To address this issue, in some examples, an outer skirt can be secured to a portion of a frame of the prosthetic heart valve including the commissure windows 242 by attaching an upper edge of the outer skirt directly to the wedge member of the commissure, such as the wedge member 420 of the commissure 454 shown in FIGS. 17-30. For example, FIG. 31 shows an exemplary example of a prosthetic heart valve 500 comprising a frame 502 (which can be similar to the frame 202 of FIG. 3, except for a configuration of its apex regions) and an outer skirt 504 arranged around an outer surface of the frame 502. The outer skirt 504 can comprise an outflow edge portion 506 (e.g., upper edge) and an inflow edge portion 508 (e.g., lower edge).

The inflow edge portion 508 can be secured to the inflow end of the frame 502 by one or more sutures 510 that are routed around struts forming the inflow end of the frame 502. The outer skirt 504 can further be secured to struts of the frame 502 extending along a cusp edge portion of the leaflets of the prosthetic heart valve by one or more sutures 516 (shown as whip stitches in FIG. 31).

The outflow edge portion 506 of the outer skirt 504 can be secured to the frame 502 by one or more sutures 512 that can extend through the lower apertures 249 in the axial struts 232 and one or more sutures 514 that extend through the outflow edge portion 506 and the commissure 454. For example, the one or more sutures 514 can extend and be looped through the wedge member 420 at the commissure 454.

Thus, the outflow edge portion 506 of the outer skirt 504 can be attached to all the axial struts of the frame 202, including the axial struts 232 and the axially extending window strut portions 240 (e.g., commissure supports), by suturing the outflow edge portion 506 to the lower apertures 249 of the axial struts 232 and to the wedge member 420 at the commissures 454 (FIGS. 31 and 32).

FIG. 32 shows an example of a prosthetic heart valve 600 comprising a frame 602 (similar to the frame 502 of FIG. 31) and an outer skirt 604. The outer skirt 604 can include a connecting suture line 610, as described above with reference to FIGS. 13A and 13B. The connecting suture line 610, at the outflow edge portion 606 of the outer skirt 604 can be secured directly to the lower apertures 249 in the axial struts 232 and to the wedge member 420 of the commissures 454 by sutures 608 (similar to as described above with reference to FIG. 31).

FIGS. 33-43 present a method for forming an integrated wedge member for a commissure of a prosthetic heart valve using one or more main sutures of a connecting suture line formed along the cusp edge portions of the leaflets. More specifically, FIGS. 33-43 present a method for forming a wedge member 702 from end portions 360 and 362 of the main sutures 310 and/or 312 of the connecting suture line 304, arranging the wedge member 702 between a pair of commissure tabs 306 extending through a commissure window 242 of a frame 436 of the prosthetic heart valve, and securing the wedge member 702 to the pair of commissure tabs 306 and upper and lower portions of the commissure window 242. As a result, a commissure 700 having an integrated wedge member 702 and having a narrower overall profile can be formed (as compared to commissures that are formed by securing portions of the commissure around the outer surfaces of the axially extending struts forming the commissure window).

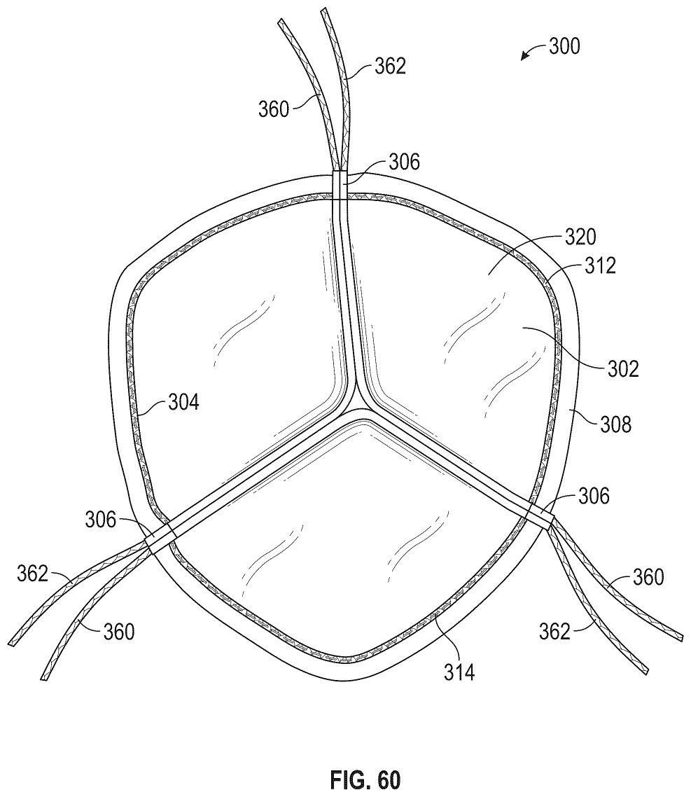

As introduced above with reference to FIGS. 4-6, and shown in FIGS. 60-63, a leaflet assembly 300 can be formed in a flat configuration outside the frame of the prosthetic heart valve. The connecting suture line 304 can extend across the cusp edge portions 308 of all the leaflets 302. In some examples, as described above and shown in FIG. 60, the connecting suture line 304 can be formed from three sutures on each of the leaflets including the first thicker (or main) suture 310 extending along the cusp edge portion 308 on one side of the leaflet 302 (e.g., the outer or smoother side 322), the second thicker (or main) suture 312 extending along the cusp edge portion 308 on the opposite side of the leaflet 302 (e.g., the inner or rougher side 320), and the stitching suture 314 extending through both the first thicker suture 310 and the second thicker suture 312. As explained above, in some examples, the same second thicker suture 312 can extend along the cusp edge portions 308 of all three leaflets 302 and individual thicker sutures 310 can extend along the cusp edge portions 308 of each of the leaflets 302 (a different one for each leaflet 302).

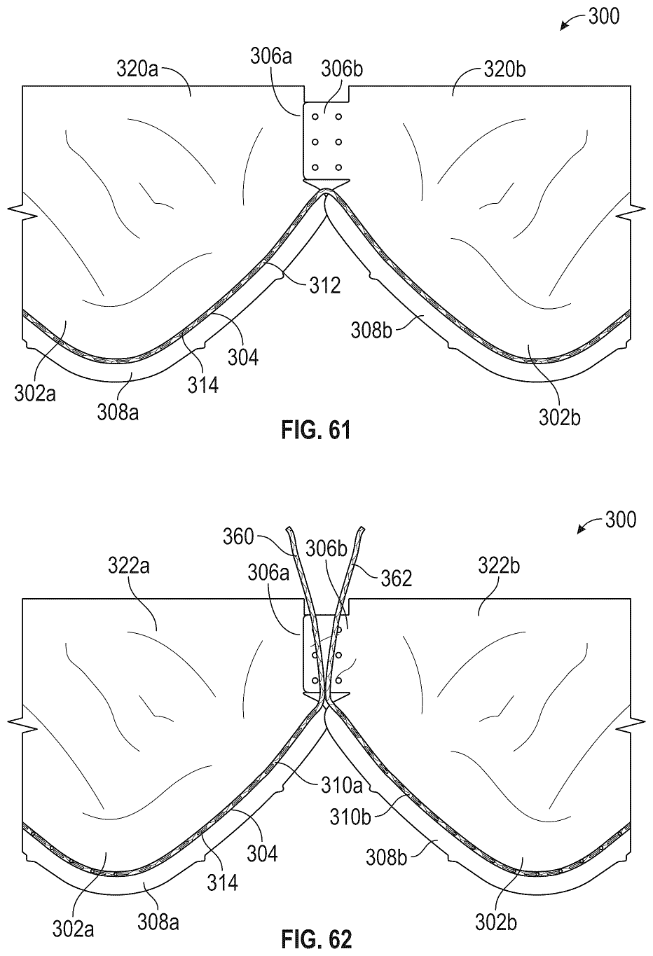

FIGS. 61 and 62 show this leaflet assembly process in more detail by showing a first leaflet 302a and a second leaflet 302b of the leaflet assembly 300 being secured together with the connecting suture line 304. As shown in FIG. 61, a single second thicker suture 312 extends along the first and second cusp edge portions 308a and 308b, on the rougher sides 320a and 320b of the first and second leaflets 302a and 302b, respectively. On the smoother sides 322a and 322b of the first and second leaflets 302a and 302b, respectively, different first sutures 310a and 310b extend along the respective cusp edge portion 308a and 308b of the first and second leaflets 302a and 302b, thereby forming two suture end portions 360 and 362 (or tails) at the commissure tabs 306a and 306b of the first and second leaflets 302a and 302b (FIG. 62).

During assembly of the leaflet assembly 300 as described herein, the two suture end portions (or tails) 360 and 362 (FIGS. 33, 60, and 62) can result at each pair of commissure tabs 306 (FIG. 60). For example, each first thicker suture 310 can extend along a cusp edge portion 308 of a corresponding leaflet 302 with end portions extending beyond ends (or end edges) of the cusp edge portion 308, thereby creating suture end portions on either end of the cusp edge portion 308 of each leaflet 302 (FIG. 60). Thus, at each pair of commissure tabs 306 for two adjacent leaflets 302, a first suture end portion 360 and a second suture end portion 362 extend from the connecting suture line 304, below the pair of commissure tabs 306 (FIG. 33 and FIG. 60).

As also introduced above with reference to FIG. 6, in some instances, the stitching suture 314 can further extend in an in-and-out pattern along both commissure tabs 306 of each leaflet 302 of the leaflet assembly 300, thereby forming the stitch line 318 along the commissure tabs 306 adjacent to an attached edge 319 of the commissure tab 306 that connects to a body 321 of the leaflet 302 (e.g., closer to the attached edge 319 than a free edge of the commissure tab 306). Thus, the in-and-out stitches 716 of the stitch line 318 can be continuous with the in-and-out stitches 315 formed with the stitching suture 314. In some examples, the portion of the stitching suture 314 forming the stitch line 318 can be tied below the commissure tabs.

After forming the leaflet assembly 300, each pair of commissure tabs 306 (formed from one commissure tab 306 from each of two adjacent leaflets 302) can pass through a commissure window 242 formed in the frame 436 (or another similar frame), as shown in FIG. 33. In some examples, the commissure window 242 can be sized to allow the pair of commissure tabs 306 tabs to pass therethrough yet retain the commissure tabs 306 in position within the commissure window 242 under slight pressure (as shown in FIG. 33).

As shown in FIG. 33, the pair of commissure tabs 306 extend outward from the commissure window 242 and the frame 436 with inner surfaces 305 of the commissure tabs 306 facing one another and the stitch lines 318 of the pair of commissure tabs 306 disposed adjacent one another along the inner surfaces 305 (e.g., adjacent to where the inner surfaces 305 of the pair of commissure tabs 306 meet). As also shown in FIG. 33, the first suture end portion 360 and the second suture end portion 362 extend below the lower end 440 of the commissure window 242 and the pair of commissure tabs 306 (toward an inflow end of the frame 436 which is disposed opposite the outflow end 460 of the frame 436 that is visible in FIG. 33).

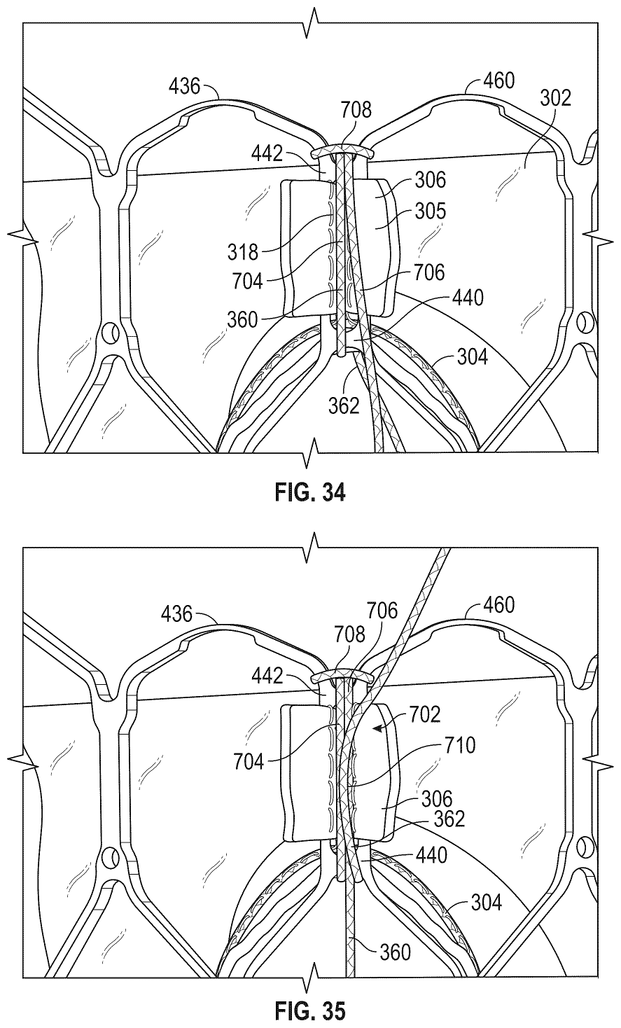

The method of forming the wedge member 702 can begin by extending the first suture end portion 360 from inside the frame 436 outward below the commissure tabs 306 and the lower end 440 (which can also be referred to as an inflow end) of the commissure window 242 (while the second suture end portion 362 remains within the frame 436). The first suture end portion 360 then extends upward (toward the outflow end 460 and the upper end 442 of the commissure window 242, which can also be referred to herein as an outflow end of the commissure window), around the lower end 440 of the commissure window 242 and between the commissure tabs 306 (across their inner surfaces 305 proximate to the stitch lines 318) (FIG. 34). The first suture end portion 360 can then be wrapped and/or tied around the upper end 442 of the commissure window 242 of the frame 436 and extended back downward between the commissure tabs 306 and toward the inflow end of the frame 436 (FIG. 34). As one example, as shown in FIG. 34, the first suture end portion 360 can extend over the upper end 442 of the commissure window 242, around a first strut that connects to the upper end 442, across the upper end 442, around a second strut that connects to the upper end 442 and back across the inner surfaces 305 of the commissure tabs 306, thereby forming a loop 708 around the frame 436 at or proximate to the upper end 442 of the commissure window 242. Thus, a first suture segment 704 and a second suture segment 706 of the first suture end portion 360 are formed between the commissure tabs 306 (FIG. 34).

The second suture end portion 362 can then be extended from inside the frame 436 outward below the lower end 440 of the commissure window 242, around the lower end 440 of the commissure window 242, and upward (toward the outflow end 460 and the upper end 442 of the commissure window 242) and between the commissure tabs 306 (across their inner surfaces 305 proximate to the stitch lines 318) (FIG. 35). This results in a third suture segment 710 overlapping the first suture segment 704 and the second suture segment 706 between the commissure tabs 306, thereby forming the wedge member 702 comprising three layers (of the main suture) (FIG. 35).

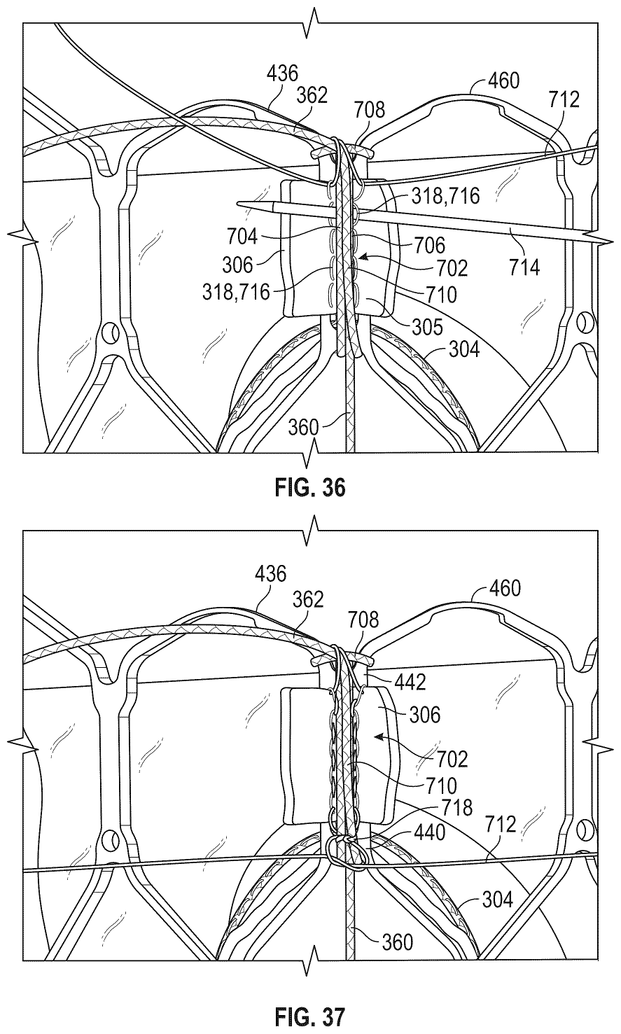

An additional suture 712 can be used to secure the wedge member 702 to the plurality of in-and-out stitches 716 of the stitch lines 318 of the commissure tabs 306, on the inner surfaces 305 of the commissure tabs 306. For examples, as shown in FIG. 36, a needle 714 (which, in some examples, can be a blunt-tipped needle) can be used to pass the suture 712 (which can be a force-fiber suture in some examples) between the inner surfaces 305 of the commissure tabs 306 and the in-and-out stitches 716 (or loops) of the in-and-out stitch lines 318 (that are disposed along the inner surfaces 305 of the commissure tabs 306), and through the wedge member 702. For example, the suture 712 can extend through one or more of (in some examples all of) the first, second, and third suture segments 704, 706, and 710 that form the wedge member 702. As a result, the wedge member 702 is secured between and to the commissure tabs 306 without the needle 714 having to puncture the tissue of the commissure tabs 306.

In some examples, the suture 712 can pass through the in-and-out stitches 716 of the in-and-out stitch lines 318 and around (e.g., looping) the wedge member 702, such that loops are found around the wedge member 702 and secure the wedge member between and to the commissure tabs 306.

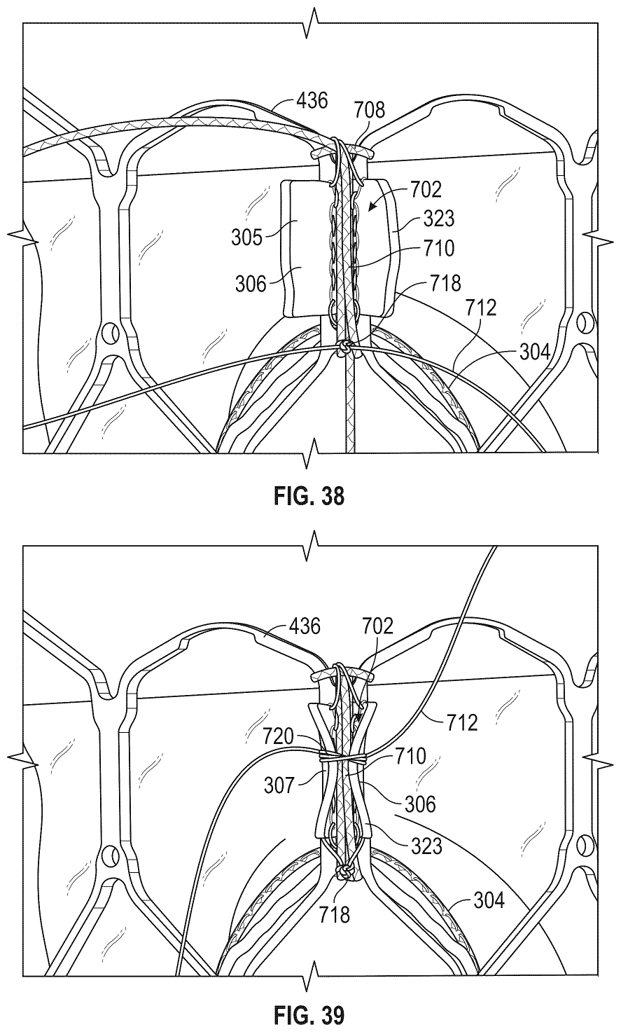

As shown in FIG. 37, after stitching the wedge member 702 to the stitch lines 318 along an axial length of the commissure tabs 306, strands (or end portions) of the suture 712 can then be tied below the commissure tabs 306, thereby forming a knot 718. The first suture end portion 360 extending below the knot 718 and the second suture end portion 362 extending above the loop 708 (FIG. 37) can then be trimmed (FIG. 38).

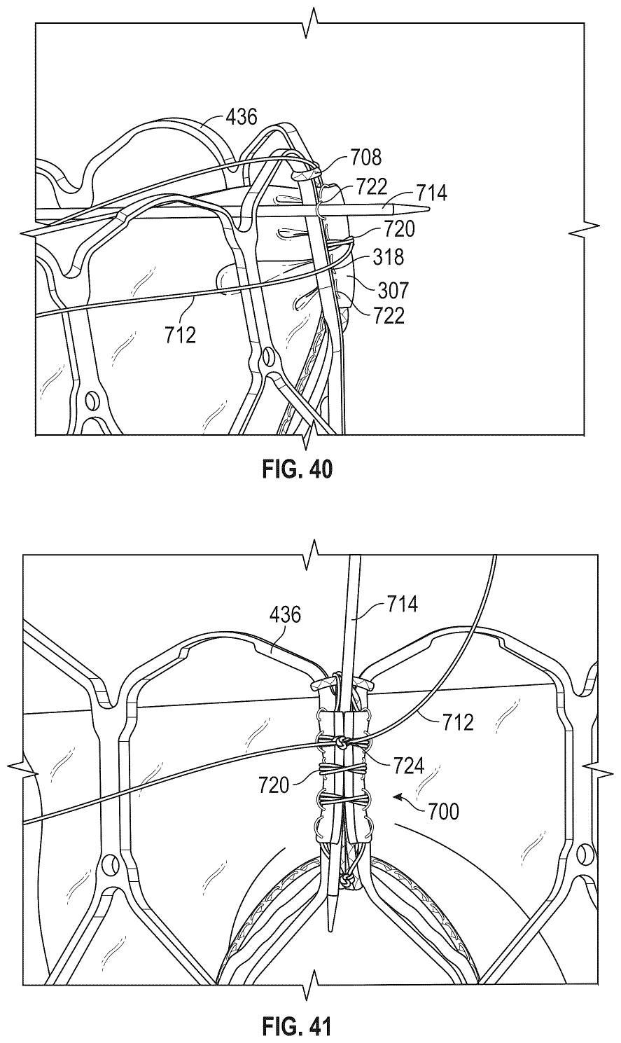

The strands of the suture 712 extending from the knot 718 cand then be tied around the commissure tabs 306 to close them over the wedge member 702, thereby forming a first loop or tie 720 around outer surfaces 307 and free (or unattached) edges 323 of the commissure tabs 306 (FIG. 39). The tails (or free end portions) of the suture 712 extending from the first tie 720 can then be passed between the in-and-out stitches 722 (or loops) of the stitch lines 318 formed along the outer surfaces 307 of the commissure tabs 306 (FIG. 40). In some examples, one or more additional ties or knots 724 can be formed around the commissure tabs 306, after passing through the stitches 722 (FIG. 41).

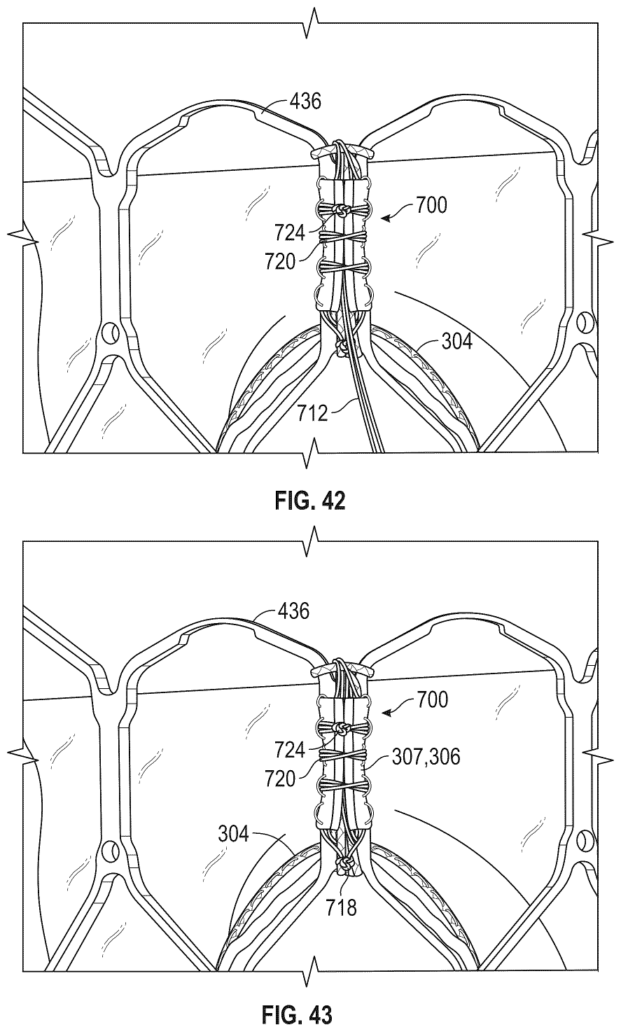

The tails or end portions of the suture 712 can then be passed with the needle 714 from above and between the commissure tabs 306 (at an outflow end of the commissure tabs 306), and through the closed commissure tabs 306 (FIG. 41), thereby containing the end portions of the suture 712 within the formed commissure 700 (FIG. 42). The tails or end portions of the suture 712 extending from the bottom (or inflow end) of the commissure 700 (FIG. 42) can then be trimmed, thereby resulting in the final configuration of the commissure 700 shown in FIG. 43.

The commissure 700 formed by the method shown in FIGS. 33-43, as described above, can be narrower, due to the wedge member 702 being contained between the commissure tabs 306 and formed from the main suture(s) of the connecting suture line 304, and the commissure 700 being attached to the upper end 442 and the lower end 440 of the commissure window 242 (instead of around the commissure window 242). The narrower commissure 700, which can have a reduced number of layers compared to commissures connected around the commissure window, can thereby create a greater amount of free space in the upper cells of the frame 436 of the prosthetic heart valve for increased coronary access (e.g., the commissure 700 may take up and block less of the free space through the cells of the frame 436 disposed on either side of the commissure window 242).

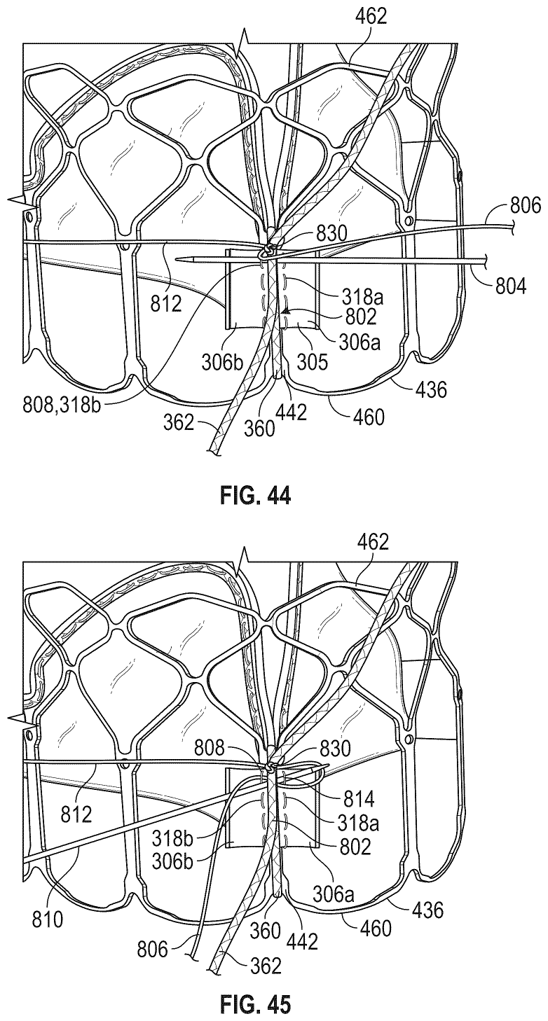

FIG. 44-59 present a method for forming an integrated wedge member for a commissure of a prosthetic heart valve using one or more main sutures of a connecting suture line formed along the cusp edge portions of the leaflets. More specifically, FIGS. 44-59 present a method for forming a wedge member 802 from first and second suture end portions 360 and 362 of the main sutures 310 and/or 312 of the connecting suture line 304, arranging the wedge member 802 between a pair of commissure tabs 306 extending through a commissure window 242 of a frame 436 of the prosthetic heart valve, and securing the wedge member 802 to the pair of commissure tabs 306 and upper and lower portions of the commissure window 242. As a result, a commissure 800 having an integrated wedge member 802 and having a narrower overall profile can be formed (as compared to commissures that are formed by securing portions of the commissure around the outer surfaces of the axially extending struts forming the commissure window).

In some examples, instead of forming the wedge member 802 from the first and second suture end portions 360 and 362, the wedge member 802 can be another integrated wedge member formed from the main sutures of a connecting suture line, such as the wedge member 420 of FIGS. 17-19B.

The method begins at FIG. 44, after extending the commissure tabs 306 through the commissure window 242 (as shown in FIG. 33). It should be noted that in the views shown in FIGS. 44-53, the frame 436 of the prosthetic heart valve is shown upside-down, such that the inflow end 462 is shown on the top of the image (in FIGS. 44-45) while the outflow end 460 is shown on the bottom of the image. Further, as used in the description below, “left” and “right” sides are defined as shown in the image views of FIGS. 44-53 (e.g., right commissure tab 306a and the left commissure tab 306b).

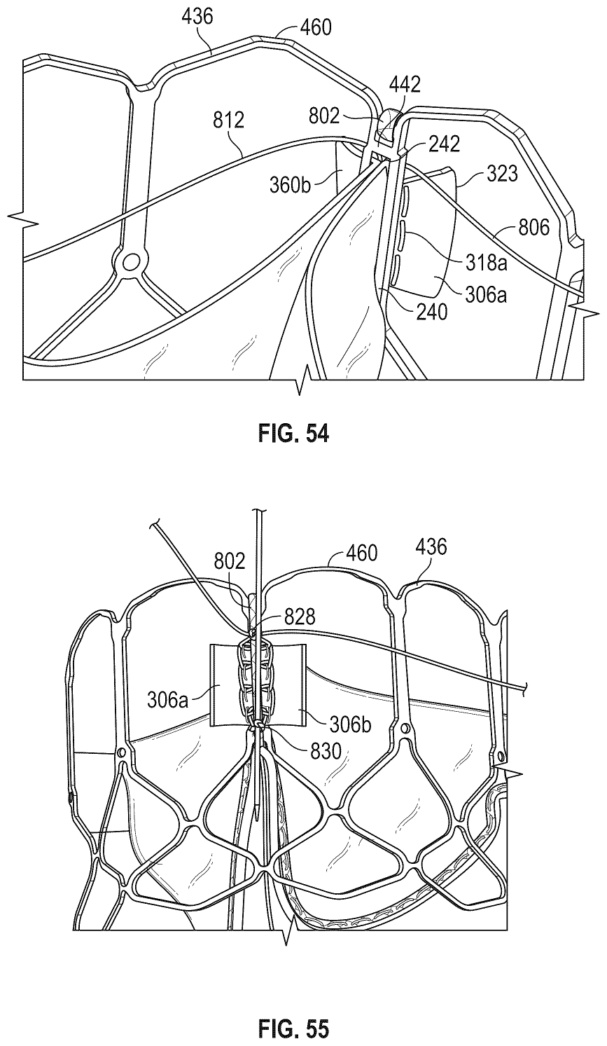

As shown in FIG. 44, the wedge member 802 has been formed across and between the commissure tabs 306, proximate to the stitch lines 318 on the inner surfaces 305 of the commissure tabs 306. In some examples, the wedge member 802 can be formed from the first suture end portion 360 and the second suture end portion 362. Further, in some examples, the wedge member can be formed from the first, second and third suture segments 704, 706, and 710 of the first suture end portion 360 and the second suture end portion 362, similar to as shown in FIGS. 34-35. In some examples, instead of looping around the upper end 442 of the commissure window 242, the first suture end portion 360 can loop around an aperture (or one or more apertures) formed in the upper end 442 (as shown in FIGS. 44 and 54).

A first knot 830 (also referred to as a lower knot) can be formed with a first suture 806 and a second suture 812 around a lower or inflow end of the wedge member 802, thereby creating suture tails or end portions of the first suture 806 and the second suture 812 extending outward form the first knot 830 (FIG. 44). As shown in FIG. 44, a first needle 804 (which can be a blunt needle in some examples) carrying the first suture 806 (which can be a force-fiber suture in some examples) is passed from right to left, though the wedge member 802 and between a first left stitch 808 (or loop) of the left stitch line 318b and the left commissure tab 306b. As used herein, a “first stitch” refers to the stitch of the particular stitch line 318 that is arranged closer to the inflow end 462 than the outflow end 460 of the frame 436 compared to the subsequent stitches of the stitch line 318.

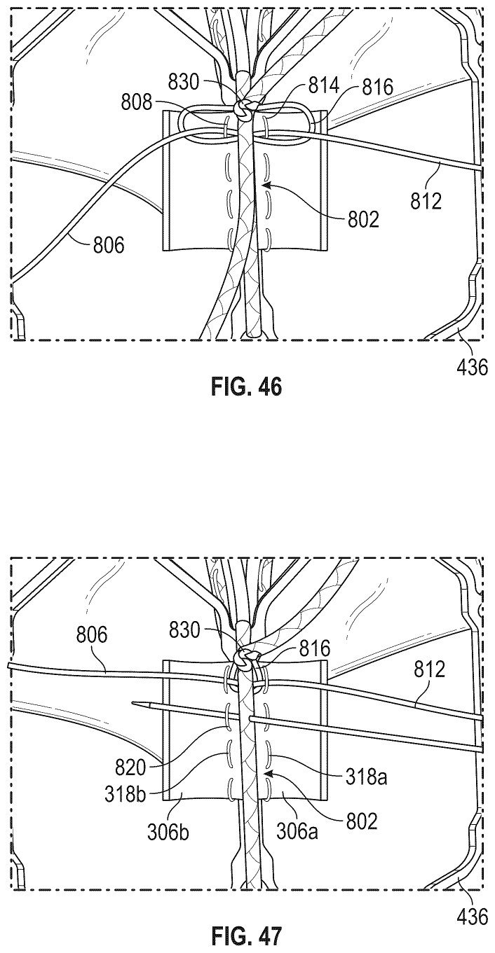

A second needle 810 carrying the second suture 812 can then be passed from left to right, through the wedge member 802 and between a first right stitch 814 of the right stitch line 318a and the right commissure tab 306a (FIG. 45). As shown in FIG. 46, this results in a loosened first loop 816. Pulling on the tails (or end portions) of both the first and second sutures 806 and 812 tightens the first loop 816, as shown in FIG. 47.

Advantageously, passing each of the first suture 806 and the second suture 812 through oppositely arranged stitches 808 and 814, instead of through the same stitches, can prevent the first and second sutures 806 and 812 from interlocking with each other.

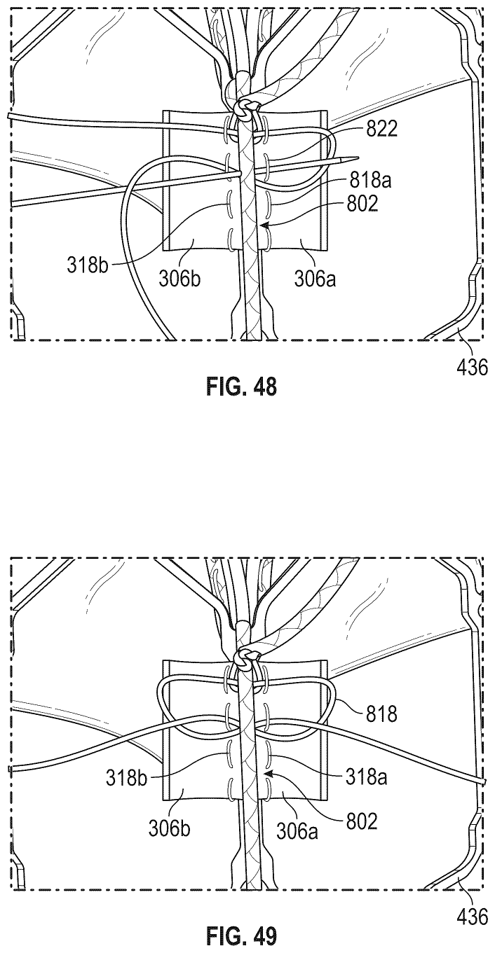

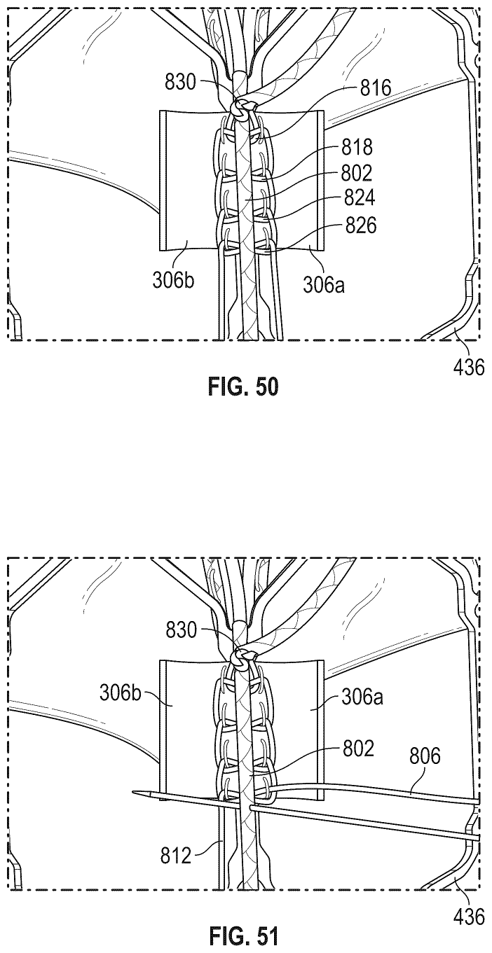

This process for forming a loop in opposing stitches of the right stitch line 318a and the left stitch line 318b can be repeated to form additional tightened loops similar to the first loop 816. For example, as shown in FIGS. 47-49, a second loop 818 can be formed by passing the second suture 812 and the first suture 806 through the oppositely arranged second left stitch 820 (FIG. 47) and second right stitch 822 (FIG. 48). The second loop 818 (FIG. 49) can then be tightened and this process can be repeated for form a third loop 824 and a fourth loop 826 (FIG. 50).

While FIG. 50 shows four loops extending between the inflow and outflow ends of the commissure tabs 306a, 306b, it is to be understood that a different number of loops (e.g., two, three, five, or the like) can be formed to secure the wedge member 802 to the stitch lines 318a, 318b of the commissure tabs 306a, 306b.

In some examples, the resulting stitch pattern formed by the loops 816, 818, 824, and 826 can resemble a macrame stitch pattern.

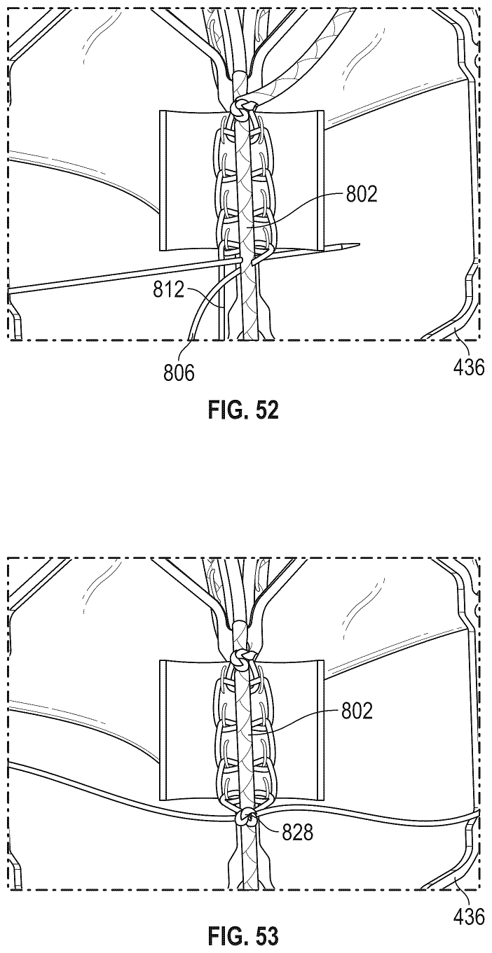

As shown in FIGS. 51 and 52, each suture tail of the first suture 806 and the second suture 812 can then be passed through a portion of the wedge member 802 disposed proximate to or above the outflow end of the commissure tabs 306a, 306b with one of the needles, and a second knot 828 (e.g., a square knot) can be formed with the suture tails (FIG. 53).

As shown from the inside or interior view of the frame 436 of the prosthetic heart valve of FIG. 54, the suture tails of the first suture 806 and the second suture 812 can extend in opposite directions above the commissure tabs 306a, 306b, proximate to the upper (or outflow) end 442 of the commissure window 242.

In some examples, at this stage the commissure tabs 306a, 306b can be trimmed along their free edges 323, thereby shortening the commissure tabs 306a, 306b.

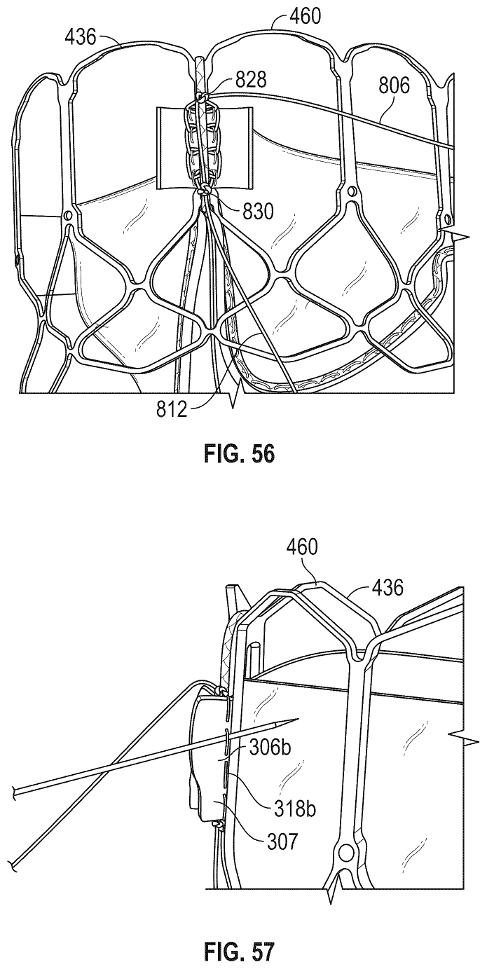

A needle carrying one of the suture tails of the first suture 806 or the second suture 812 (e.g., the first needle 804 carrying the first suture 806 or the second needle 810 carrying the second suture 812) can then be passed through the first knot 830 (FIG. 55), thereby resulting in the configuration shown in FIG. 56 with one of the sutures (e.g., first suture 806) extending from the second knot 828 above an outflow end of the commissure tabs 306a, 306b and another one of the sutures (e.g., second suture 812) extending from the first knot 830 beyond an inflow end of the commissure tabs 306a, 306b.

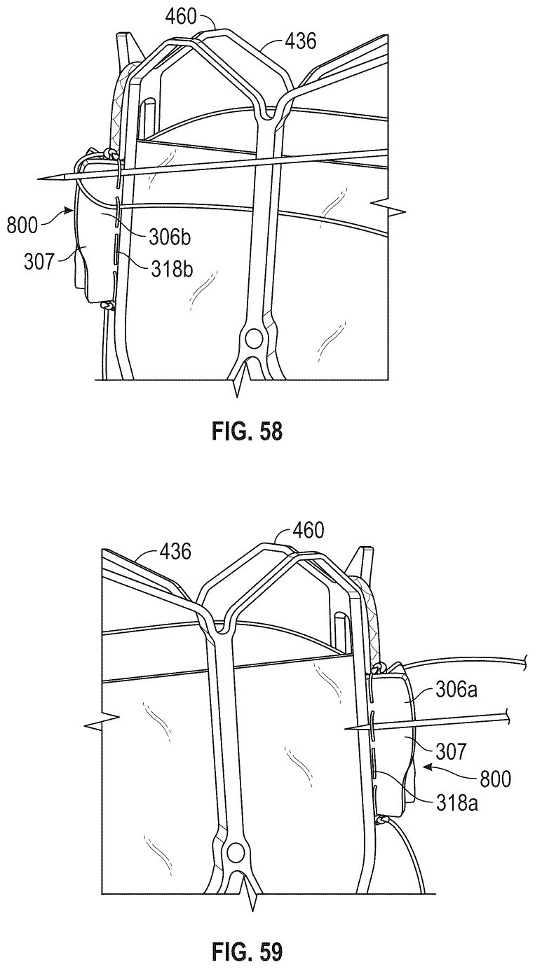

From this point onward, the commissure tabs 306a, 306n can be folded toward each other and the first suture 806 and/or the second suture 812 can be utilized to extend through the stitches of the left stitch line 318b along the outer surface 307 of the left commissure tab 306b (FIGS. 57 and 58) and through the stitches of the right stitch line 318a along the outer surface 307 of the right commissure tab 306a (FIG. 59), similar to as described above with reference to FIG. 40. The commissure 800 can then be further formed and finished by closing the commissure tabs 306a, 306b together with one or more knots and securing end portion of the sutures through an interior of the commissure 800, as described above with reference to FIGS. 41-43

The stitch pattern described above with reference to FIGS. 44-53 can secure the wedge member 802 to the commissure tabs 306a, 306b in a way that occupies a smaller footprint, thereby enabling the commissure 800 to assume a relatively narrow final profile. Further, trimming the commissure tabs 306a, 306b as described above can serve to further narrow the final commissure profile, thereby resulting in a commissure size that frees a larger portion of the upper cells of the frame of the prosthetic heart valve for coronary access (e.g., increased blood flow through the cells of the frame).

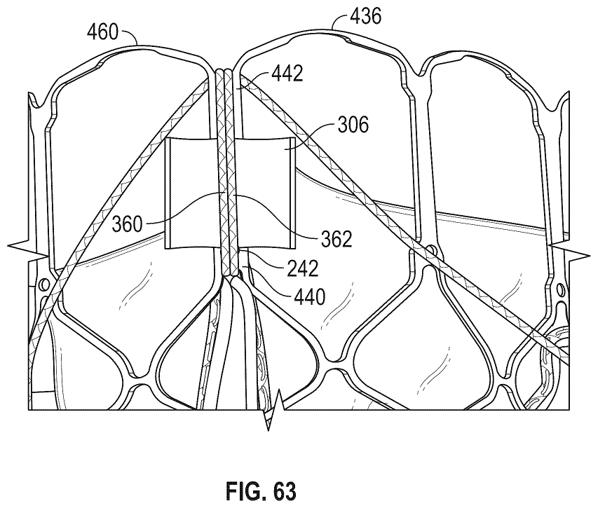

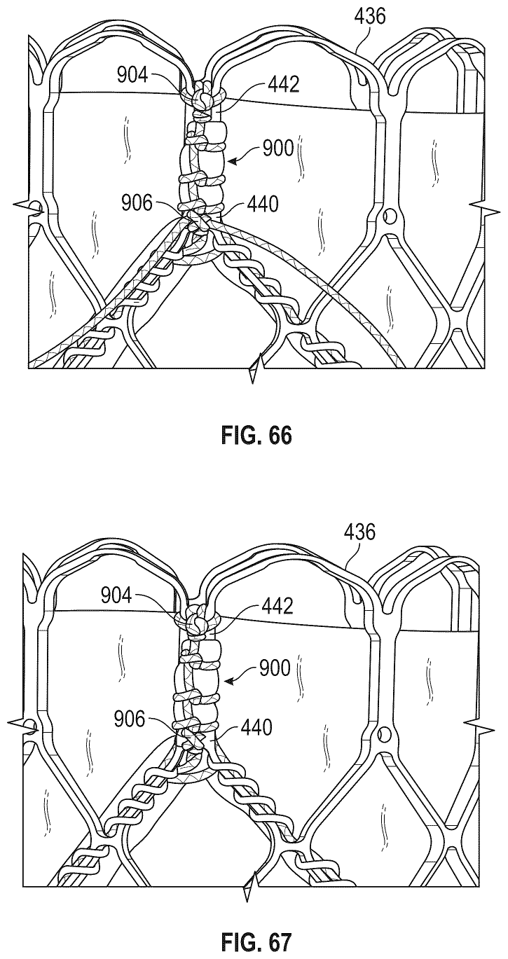

FIGS. 63-67 present a method for forming an integrated wedge member for a commissure of a prosthetic heart valve using one or more main sutures of a connecting suture line formed along the cusp edge portions of the leaflets. More specifically, FIGS. 63-67 present a method for forming a wedge member 902 (FIG. 65) from first and second suture end portions 360 and 362 (FIG. 63) of the main sutures 310 and/or 312 of the connecting suture line 304 (e.g., as described above with reference to FIGS. 60-62), arranging the wedge member 902 between a pair of commissure tabs 306 extending through a commissure window 242 of a frame 436 of the prosthetic heart valve, and securing the wedge member 902 to the pair of commissure tabs 306 and upper and lower portions of the commissure window 242. As a result, a commissure 900 having an integrated wedge member 902 can be formed (FIGS. 66 and 67).

The method begins at FIG. 63, after extending the commissure tabs 306 through the commissure window 242 (as shown in FIG. 33). The first suture end portion 360 (tail) and the second suture end portion 362 (tail) are passed from inside the frame 436 outward below the commissure tabs 306 and the lower end 440 (which can also be referred to as an inflow end) of the commissure window 242. The first suture end portion 360 and the second suture end portion 362 then extend upward (toward the outflow end 460 and the outflow or upper end 442 of the commissure window 242), around the lower end 440 of the commissure window 242, between the commissure tabs 306 (across their inner surfaces 305 proximate to the stitch lines 318), and over the outflow or upper end 442 of the commissure window 242 (FIG. 63).

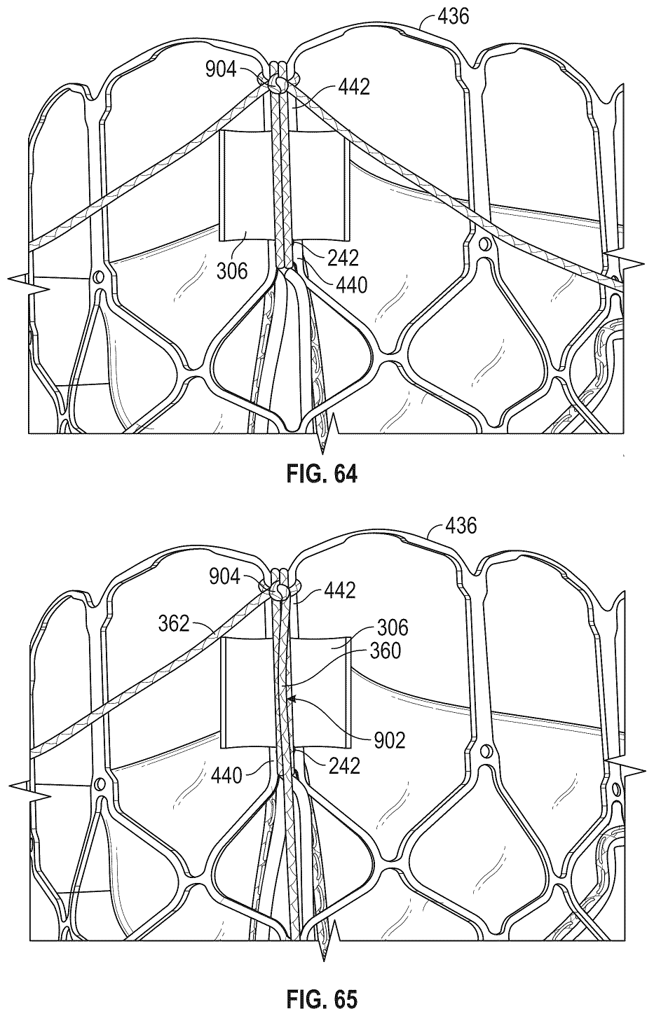

The first suture end portion 360 and the second suture end portion 362 can then be wrapped and/or tied around the upper end 442 of the commissure window 242 of the frame 436, thereby forming a first knot 904 (FIG. 64). In some examples, the first suture end portion 360 can then be extended back downward between the commissure tabs 306 and toward the inflow end of the frame 436 (FIG. 65) and can then be tied off into a second knot 906 around the lower end 440 of the commissure window 242. Thus, the resulting wedge member 902 can be formed from three overlapping suture segments of the first suture end portion 360 and the second suture end portion 362, thereby making the wedge member 902 three suture-widths thick.

Alternatively, in some examples, the first suture end portion 360 and the second suture end portion 362 can be extended back downward between the commissure tabs 306 and toward the inflow end of the frame 436 and can then be tied together around the lower end 440 of the commissure window 242, thereby forming the second knot 906 (FIG. 66). In such instances, the resulting wedge member 902 can be formed from four overlapping suture segments of the first suture end portion 360 and the second suture end portion 362, thereby making the wedge member 902 four suture-widths thick.

The commissure tabs 306 can then be secured together around the wedge member 902 using any of the methods described herein, such as by using a blanket stitch pattern (e.g., as shown in FIGS. 26-30). A schematic depicting a cross-sectional view of a wedge member 950 being secured to the commissure tabs 306 with a whip or blanket stitch pattern is shown in FIG. 72. Specifically, as described herein, the wedge member 950 (which can be any of the wedge members described herein) can be secured to the commissure tabs 306 by a plurality of in-and-out stitches 952 and/or a series of whip or blanket stitches 954 which extend through the wedge member 950 and through and around the front of the commissure tabs 306 that are wrapped around the wedge member 950 (FIG. 72).

Returning to FIG. 67, the ends of the first suture end portion 360 and the second suture end portion 362 can be trimmed, thereby forming the final commissure 900 shown in FIG. 67.

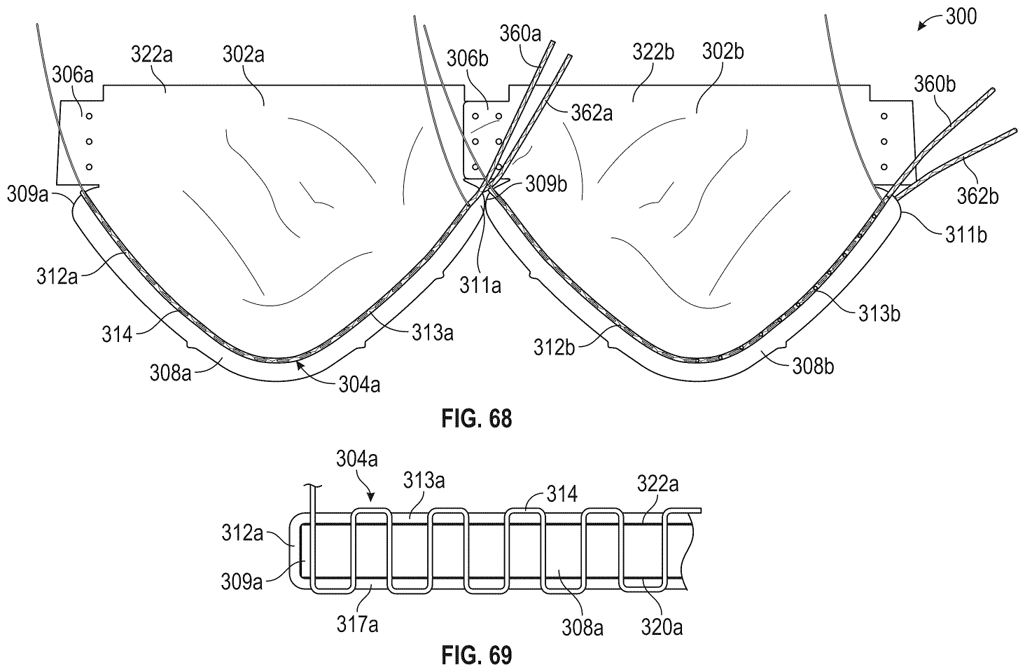

Turning now to FIGS. 68 and 69, a method for forming suture end portions 360 and 362 for forming a wedge member using one or more sutures of the connecting suture line 304 is shown. In particular, FIG. 68 shows two leaflets 302a and 302b of the leaflet assembly 300 disposed adjacent one another where one main or thicker suture is used to form the connecting suture line for each leaflet. FIG. 69 shows a cross-sectional view of a portion of the first leaflet 302a.

As shown in FIGS. 68 and 69, a first thicker suture 312a can be folded (e.g., in half) around a first end 309a of the cusp edge portion 308a of the first leaflet 302a such that a first portion 313a of the first thicker suture 312a extends along the smoother side 322a of the first leaflet 302a and a second portion 317a of the first thicker suture 312a extends along the opposite, rougher side 320a of the first leaflet 302a (FIG. 69), thereby resulting in suture end portions 360a and 362a extending away from a second end 311a of the cusp edge portion 308a (FIG. 68). The suture end portions 360a and 362a can then be used to form the wedge member at the commissure between the first leaflet 302a and the second leaflet 302b. As described previously, the thinner suture 314 can extend through the first and second portions 313a and 317a of the first thicker suture 312a and the cusp edge portion 308a of the first leaflet 302a, thereby forming the connecting suture line 304a.

Similarly, a second thicker suture 312b can be folded (e.g., in half) around a first end 309b of the cusp edge portion 308b of the second leaflet 302b such that a first portion 313b of the first thicker suture 312a extends along the smoother side 322b of the second leaflet 302b and a second portion of the first thicker suture 312b extends along the opposite, rougher side of the second leaflet 302b, thereby resulting in suture end portions 360b and 362b extending away from a second end 311b of the cusp edge portion 308b. The suture end portions 360b and 362b can then be used to form the wedge member at the commissure between the second leaflet 302b and a third leaflet of the leaflet assembly 300. By forming the suture end portions 360a, 362a, 360b, 362b as shown in FIGS. 68 and 69, the suture end portions can be relatively long (without having to be pre-measured) and can then be cut to the desired length after formation of the wedge member.

The suture end portions 360a and 362a (or 360b and 362b) can be used in lieu of any of the suture end portions described above, with respect to any of the commissure formation methods described herein.

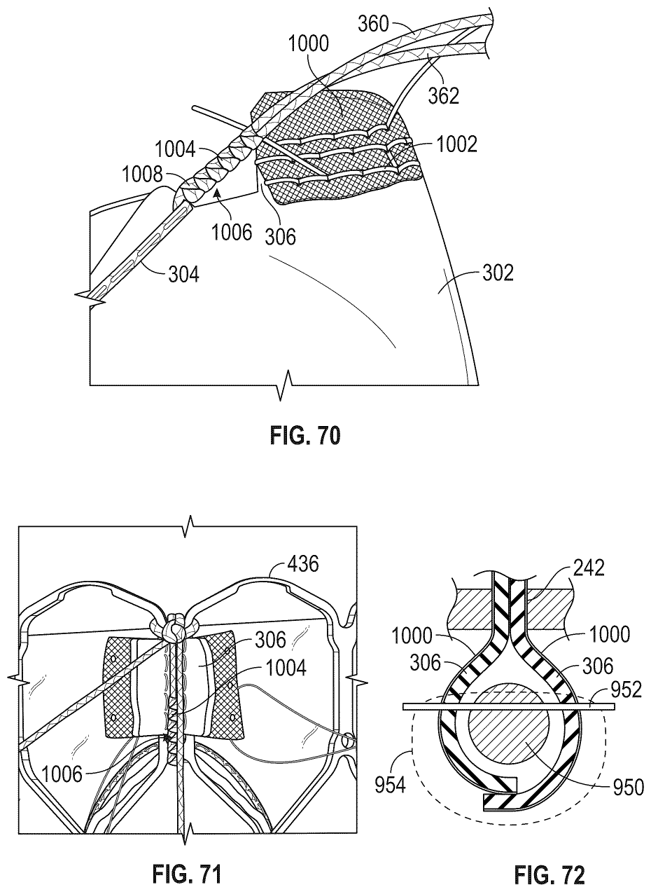

In some examples, as shown in FIGS. 70-72, a fabric post 1000 can be secured to the commissure tabs 306 of the leaflets, prior to forming the commissure. For example, in any of the wedge member and commissure formation methods described herein, a fabric post 1000 can first be secured (e.g., stitched) to a first side (e.g., an outer side, as shown in FIG. 71) of the commissure tabs 306 via a plurality of stitches 1002, prior to securing the wedge member to the commissure tabs 306. The fabric post 1000 can comprise a fabric material (e.g., such as a textile material) that is configured to help retain the commissure tabs 306 within the commissure window 242.

FIGS. 70 and 71 show a way of forming a wedge member 1004 from suture end portions 360 and 362 (which, in some instances, can be the suture end portions 360a and 362a or 360b and 362b from FIG. 68). The suture end portions 360 and 362 can be stitched together (e.g., one through the other) to form a thicker portion 1006 of the wedge member 1004, which can consist of several in-and-out stitches 1008 (e.g., 4-5) (FIG. 70), and the suture end portions 360 and 362 extending from the thicker portion 1006. The thicker portion 1006 of the wedge member 1004 can then be extended across the inner sides of the commissure tabs 306, as shown in FIG. 71. The suture end portions 360 and 362 can then be tied around the upper end 442 of the commissure window 242 and used to form a complete commissure, as described above with reference to FIGS. 63-67 (except the blanket and in-and-out stitches can also extend through the fabric posts 1000, as shown in FIGS. 71 and 72).

In some examples, the wedge member 1004 can be used as the wedge member in any of the other commissure formation methods described herein.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (e.g., by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic heart valve comprising: a frame comprising a plurality of interconnected struts; a plurality of leaflets disposed within the frame and configured to regulate a flow of blood through the frame in one direction, each leaflet comprising a cusp edge portion; and at least one suture extending along a cusp edge portion of at least a first leaflet of the plurality of leaflets, a plurality of in-and-out stitches extending through the suture and the cusp edge portion of the first leaflet, and a plurality of whip stitches that extend around selected struts of the plurality of the interconnected struts and the suture to couple the cusp edge portion of the first leaflet to the frame.

Example 2. The prosthetic heart valve of any example herein, particularly example 1, wherein the at least one suture comprises first and second sutures disposed on opposite sides of the cusp edge portion of the first leaflet, and the in-and-out stitches extend through the first suture, the cusp edge portion of the first leaflet, and the second suture.

Example 3. The prosthetic heart valve of any example herein, particularly example 1, wherein the at least one suture comprises a first suture folded over a first end of the cusp edge portion of the first leaflet with a first portion of the first suture extending along a first side of the first leaflet to a second end of the cusp edge portion and a second portion of the first suture extending along an opposite, second side of the first leaflet to the second end of the cusp edge portion, and wherein the in-and-out stitches extend through the first portion of the first suture, the cusp edge portion of the first leaflet, and the second portion of the first suture.

Example 4. The prosthetic heart valve of any example herein, particularly any one of examples 1-3, wherein the whip stitches extend between the suture and the cusp edge portion of the first leaflet without extending through the first leaflet.

Example 5. The prosthetic heart valve of any example herein, particularly any one of examples 1-3, wherein the whip stitches extend between the suture and the plurality of in-and-out stitches without extending through the first leaflet.

Example 6. The prosthetic heart valve of any example herein, particularly any one of examples 1-3, wherein the whip stitches extend through the suture without extending through the first leaflet.

Example 7. The prosthetic heart valve of any example herein, particularly any one of examples 1-6, wherein the at least one suture extends along the cusp edge portion of each leaflet and is secured thereto with the in-and-out stitches and the whip stitches couple the cusp edge portion of each leaflet to selected struts of the plurality of interconnected struts of the frame.

Example 8. The prosthetic heart valve of any example herein, particularly any one of examples 1-7, wherein the frame comprises a plurality of commissure supports, and each leaflet comprises opposing commissure tabs, wherein each commissure tab is paired with an adjacent commissure tab of an adjacent leaflet, and each pair of commissure tabs extends radially through an opening in a corresponding commissure support.

Example 9. The prosthetic heart valve of any example herein, particularly example 8, wherein the at least one suture forms at least one wedge member that extends between the commissure tabs of at least a first pair of commissure tabs on an outside of the frame.

Example 10. The prosthetic heart valve of any example herein, particularly example 9, further comprising a plurality of additional stitches that secure the at least one wedge member to the commissure tabs of the first pair of commissure tabs.

Example 11. The prosthetic heart valve of any example herein, particularly example 10, wherein the plurality of additional stitches also secures the at least one wedge member to the corresponding commissure support, above and below the opening in the corresponding commissure support.

Example 12. The prosthetic heart valve of any example herein, particularly any one of examples 9-11, wherein the at least one wedge member is formed by two or more overlapping portions of the at least one suture such that a first thickness of the at least one wedge member is thicker than a second thickness of the at least one suture.

Example 13. The prosthetic heart valve of any example herein, particularly any one of examples 1-12, wherein the at least one suture is thicker than a thickness of the in-and-out stitches.

Example 14. The prosthetic heart valve of any example herein, particularly any one of examples 1-13, further comprising an outer skirt disposed around an outer surface of the frame and another plurality of in-and-out stitches extending through the outer skirt along an edge of the outer skirt, and wherein the plurality of whip stitches further extend around the plurality of in-and-out stitches in the outer skirt to couple the outer skirt to the frame.

Example 15. A leaflet assembly for a prosthetic heart valve, comprising: a plurality of leaflets, each leaflet comprising opposing commissure tabs disposed on opposite sides of the leaflet and a cusp edge portion extending between the opposing commissure tabs; a connecting suture line extending along the cusp edge portion of each leaflet and securing the plurality of leaflets together, the connecting suture line including one or more first sutures arranged along the cusp edge portion on at least one side of each leaflet and one or more second sutures stitched in a running-stitch pattern through the one or more first sutures and through each leaflet; and a plurality of wedge members formed from the one or more first sutures, each wedge member extending from the cusp edge portions of two adjacent leaflets of the plurality of leaflets and between two commissure tabs of the two adjacent leaflets.

Example 16. The leaflet assembly of any example herein, particularly example 15, wherein each wedge member comprises at least two layers of the one or more first sutures such that a width of the wedge member is at least two times thicker than a thickness of the one or more first sutures.

Example 17. The leaflet assembly of any example herein, particularly example 15 or example 16, wherein each wedge member comprises a first portion of the one or more first sutures that extends along the two commissure tabs, loops around at a top of the two commissure tabs, and is threaded through a second portion of the one or more first sutures such that that the wedge member comprises a loop at a first end of the wedge member and has an increased thickness relative to a thickness of the one or more first sutures between the two commissure tabs.

Example 18. The leaflet assembly of any example herein, particularly example 17, wherein first and second suture segments of the one or more first sutures extend from a second end of each wedge member and wherein the first suture segment extends along the cusp edge portion of a first leaflet of the plurality of leaflets and the second suture segment extends along the cusp edge portion of an adjacent, second leaflet of the plurality of leaflets.

Example 19. The leaflet assembly of any example herein, particularly example 17, wherein first and second suture segments of the one or more first sutures extend from a second end of each wedge member and wherein the first suture segment extends along the cusp edge portion on a first side of a first leaflet of the plurality of leaflets and the second suture segment extends along the cusp edge portion of a second side of the first leaflet, and wherein the one or more second sutures extend through the first suture segment, the cusp edge portion of the first leaflet, and the second suture segment.

Example 20. The leaflet assembly of any example herein, particularly any one of examples 15-19, wherein the one or more first sutures have a first thickness and the one or more second sutures have a second thickness, the first thickness greater than the second thickness.

Example 21. The leaflet assembly of any example herein, particularly any one of examples 15-20, further comprising a plurality of in-and-out stitches formed along the opposing commissure tabs of each leaflet and extending through apertures pre-formed in the opposing commissure tabs.

Example 22. The leaflet assembly of any example herein, particularly any one of examples 15-21, wherein the cusp edge portion of each leaflet comprises a plurality of apertures disposed therein and wherein the one or more second sutures extend through the plurality of apertures.

Example 23. A prosthetic heart valve comprising: a frame comprising a plurality of interconnected struts and a plurality of commissure supports defining respective commissure windows; a plurality of leaflets, each leaflet comprising opposing commissure tabs disposed on opposite sides of the leaflet and a cusp edge portion extending between the opposing commissure tabs, wherein the plurality of leaflets are connected together and to the frame by a connecting suture line extending along the cusp edge portions of the leaflets, the connecting suture line comprising one or more first sutures arranged along the cusp edge portion on at least one side of each leaflet and one or more second sutures stitched in an in-and-out pattern through the leaflets and the one or more first sutures; and a plurality of commissures secured to the plurality of commissure supports, each commissure comprising two commissure tabs, one from each of two adjacent leaflets of the plurality of leaflets, and a wedge member extending between and along the two commissure tabs on an exterior of a corresponding commissure support of the frame, the wedge member comprising two or more overlapping portions of the one or more first sutures of the connecting suture line.

Example 24. The prosthetic heart valve of any example herein, particularly example 23, wherein for each commissure, the two commissure tabs are secured to the wedge member and the corresponding commissure support by one or more third sutures.

Example 25. The prosthetic heart valve of any example herein, particularly example 24, wherein the one or more third sutures extend through pre-formed apertures in the two commissure tabs and through the wedge member.

Example 26. The prosthetic heart valve of any example herein, particularly any one of examples 23-25, wherein the one or more first sutures have a first thickness and the one or more second sutures have a second thickness, the first thickness greater than the second thickness

Example 27. The prosthetic heart valve of any example herein, particularly any one of examples 23-26, wherein the wedge member comprises two overlapping portions of the one or more first sutures and wherein a first portion of the two overlapping portions is folded over and threaded through a second portion of the two overlapping portions such that a loop is formed at a top of the wedge member.

Example 28. The prosthetic heart valve of any example herein, particularly any one of examples 23-26, wherein the one or more first sutures comprises two first sutures disposed on opposite sides of the cusp edge portion of each leaflet, and the one or more second sutures extend through the two first sutures and the cusp edge portion of each leaflet.

Example 29. The prosthetic heart valve of any example herein, particularly any one of examples 23-26, wherein the one or more first sutures comprises a single first suture arranged along the cusp edge portions on a first side of all leaflets of the plurality of leaflets and three individual first sutures, each of the three individual first sutures arranged along the cusp edge portion of a second side of a corresponding leaflet of the plurality of leaflets, and wherein the wedge member is formed by free end portions of one of the three individual first sutures.

Example 30. The prosthetic heart valve of any example herein, particularly any one of examples 23-26, wherein the one or more first sutures comprises three individual first sutures, each individual first suture folded over a first end of the cusp edge portion of a corresponding leaflet with a first portion of the individual first suture extending along a first side of the corresponding leaflet and a second portion of the individual first suture extending a long an opposite, second side of the corresponding leaflet, and wherein the wedge member is formed by free end portions of the first portion and the second portion of the individual first suture at a second end of the cusp edge portion of the corresponding leaflet.

Example 31. The prosthetic heart valve of any example herein, particularly any one of examples 23-30, wherein the cusp edge portion of each leaflet of the plurality of leaflets is attached to selected struts of the plurality of interconnected struts by a plurality of whip stitches that extend around the selected struts and around or through the connecting suture line.

Example 32. The prosthetic heart valve of any example herein, particularly example 31, wherein the whip stitches extend between one first suture of the one or more first sutures and the cusp edge portion of each leaflet without extending through the leaflet.

Example 33. The prosthetic heart valve of any example herein, particularly example 31, wherein the whip stitches extend between one first suture of the one or more first sutures and one second suture of the one or more second sutures without extending through the leaflet.

Example 34. The prosthetic heart valve of any example herein, particularly example 31, wherein the whip stitches extend through one first suture of the one or more first sutures without extending through the leaflet.

Example 35. The prosthetic heart valve of any example herein, particularly any one of examples 31-34, further comprising an outer skirt disposed around an outer surface of the frame and a plurality of in-and-out stitches extending through the outer skirt along an edge of the outer skirt, and wherein the plurality of whip stitches further extend around the plurality of in-and-out stitches in the outer skirt to couple the outer skirt to the frame.

Example 36. The prosthetic heart valve of any example herein, particularly any one of examples 23-35, further comprising an outer skirt disposed around an outer surface of the frame, the outer skirt comprising an outflow edge and an inflow edge, the outflow edge attached to the plurality of commissures by one or more stitches that extend through the outer skirt and the wedge member of each commissure.

Example 37. The prosthetic heart valve of any example herein, particularly example 36, wherein the plurality of interconnected struts includes a plurality of axial struts, wherein one or more axial struts of the plurality of axial struts are disposed between adjacent commissure supports of the plurality of commissure supports, and wherein the outflow edge of the outer skirt is further attached to lower apertures in the axial struts.

Example 38. A method for forming a leaflet assembly for a prosthetic heart valve, comprising: securing a plurality of leaflets together into a leaflet assembly, off a frame of the prosthetic heart valve, by arranging one or more sutures along a cusp edge portion on at least one side of each leaflet of the plurality of leaflets and extending a plurality of in-and-out stitches through the one or more sutures and the plurality of leaflets, along the cusp edge portion of each leaflet; and pairing each commissure tab of each leaflet of the plurality of leaflets with an adjacent commissure tab of an adjacent leaflet, and for each pair of commissure tabs, extending a portion of the one or more sutures between the pair of commissure tabs and overlapping the portion with itself to form an integrated wedge member with a thickness that is greater than a thickness of the one or more sutures.

Example 39. The method of any example herein, particularly example 38, wherein the securing the plurality of leaflets together into the leaflet assembly is performed automatically or manually while the leaflets are arranged in a flattened configuration off the frame.

Example 40. The method of any example herein, particularly example 38 or example 39, wherein arranging the one or more sutures along the cusp edge portion on at least one side of each leaflet of the plurality of leaflets includes arranging first and second sutures of the one or more sutures on opposite sides of the cusp edge portion of each leaflet, and extending the plurality of in-and-out stitches through the first suture, the cusp edge portion of each leaflet, and the second suture.

Example 41. The method of any example herein, particularly any one of examples 38-40, further comprising arranging the plurality of leaflets within an interior of the frame of the prosthetic heart valve and attaching the cusp edge portion of each leaflet to the frame by extending a plurality of whip stitches around selected struts of the frame and the one or more sutures, without extending the plurality of whip stitches through the leaflet.

Example 42. The method of any example herein, particularly example 41, wherein extending the plurality of whip stitches around the selected struts of the frame and the one or more sutures includes extending the plurality of whip stitches between the one or more sutures and the cusp edge portion of each leaflet.

Example 43. The method of any example herein, particularly example 41, wherein extending the plurality of whip stitches around the selected struts of the frame and the one or more sutures includes extending the plurality of whip stitches between the one or more sutures and the plurality of in-and-out stitches.

Example 44. The method of any example herein, particularly example 41, wherein extending the plurality of whip stitches around the selected struts of the frame and the one or more sutures includes extending the plurality of whip stitches through the one or more sutures.

Example 45. The method of any example herein, particularly any one of examples 41-44, further comprising, for each pair of commissure tabs, extending the pair of commissure tabs radially through a corresponding commissure window formed by a corresponding commissure support portion of the frame, extending the wedge member between the pair of commissure tabs on an exterior of the corresponding commissure window, and securing the pair of commissure tabs to the wedge member and the corresponding commissure support portion to form a commissure such that the wedge member prevents the commissure from being pulled back through the corresponding commissure window.

Example 46. The method of any example herein, particularly example 45, wherein extending the portion of the one or more sutures between the pair of commissure tabs and overlapping the portion with itself to form the integrated wedge member includes threading the portion of the one or more sutures through itself to form the integrated wedge member with a loop disposed at a free end of the wedge member and wherein securing the pair of commissure tabs to the wedge member and the corresponding commissure support portion includes extending an additional suture through the loop of the wedge member and knotting the wedge member to the corresponding commissure support portion.

Example 47. The method of any example herein, particularly any one of examples 38-46, wherein the thickness of the wedge member is at least two times the thickness of the one or more sutures.

Example 48. A method comprising: attaching a connecting suture line formed along cusp edge portions of a plurality of leaflets of a leaflet assembly to struts of a frame of a prosthetic heart valve by extending a plurality of whip stitches around or through at least a portion of the connecting suture line and around selected struts of the frame to secure the cusp edge portions of the leaflets directly to the frame without extending the whip stitches through a tissue of the leaflets, wherein the connecting suture line comprises a suture disposed on the cusp edge portion of at least one side of each leaflet of the plurality of leaflets and a plurality of in-and-out stitches extending through the suture and the cusp edge portion of each leaflet; and attaching pairs of commissure tabs of adjacent leaflets of the leaflet assembly to commissure windows of the frame by extending each pair of commissure tabs radially through a corresponding commissure window formed by a corresponding commissure support portion of the frame and securing a wedge member integrated with the connecting suture line to the pair of commissure tabs on an exterior of the commissure window, the wedge member formed by two or more overlapping portions of the suture.

Example 49. The method of any example herein, particularly example 48, wherein securing the wedge member to the pair of commissure tabs includes extending the wedge member from cusp edge portions of the adjacent leaflets that comprise the pair of commissure tabs, between the pair of commissure tabs, and then securing the pair of commissure tabs to the wedge member and the corresponding commissure support portion.

Example 50. The method of any example herein, particularly example 48 or example 49, wherein the securing the wedge member to the pair of commissure tabs includes extending another suture through pre-formed apertures in the pair of commissure tabs and through the wedge member.

Example 51. The method of any example herein, particularly any one of examples 48-50, further comprising arranging an outer skirt around an outer surface of the frame and securing the outer skirt to the selected struts of the frame with the plurality of whip stitches.

Example 52. The method of any example herein, particularly example 51, wherein securing the outer skirt to the selected struts of the frame with the plurality of whip stitches includes extending the plurality of whip stitches around the selected struts, around or through at least the portion of the connecting suture line, and around another plurality of in-and-out stitches formed in the outer skirt.

Example 53. The method of any example herein, particularly example 51 or example 52, further comprising securing an outflow edge of the outer skirt to the wedge member of each pair of commissure tabs.

Example 54. The method of any example herein, particularly any one of examples 48-53, wherein extending the plurality of whip stitches around or through at least the portion of the connecting suture line and around the selected struts of the frame includes extending the plurality of whip stitches between the suture and the cusp edge portion of each leaflet.

Example 55. The method of any example herein, particularly any one of examples 48-53, wherein extending the plurality of whip stitches around or through at least the portion of the connecting suture line and around the selected struts of the frame includes extending the plurality of whip stitches between the suture and the plurality of in-and-out stitches.

Example 56. The method of any example herein, particularly any one of examples 48-53, wherein extending the plurality of whip stitches around or through at least the portion of the connecting suture line and around the selected struts of the frame includes extending the plurality of whip stitches through the suture.

Example 57. The method of any example herein, particularly any one of examples 48-56, wherein a thickness of the wedge member is at least two times a thickness of the suture.

Example 58. The method of any example herein, particularly any one of examples 48-57, wherein the suture has a first thickness that is greater than a second thickness of the plurality of in-and-out stitches.

Example 59. The method of any example herein, particularly any one of examples 48, 49, or 51, wherein the plurality of in-and-out stitches extending through the suture and the cusp edge portion of each leaflet is a first plurality of in-and-out stitches, and wherein the securing the wedge member to the pair of commissure tabs includes extending another suture through the wedge member and a second plurality of in-and-out stitches extending along and through each commissure tab of each pair of commissure tabs.

Example 60. The method of any example herein, particularly example 59, wherein the second plurality of in-and-out stitches are continuous with the first plurality of in-and-out stitches.

Example 61. A method comprising: attaching a connecting suture line formed along cusp edge portions of a plurality of leaflets of a leaflet assembly to struts of a frame of a prosthetic heart valve, wherein the connecting suture line comprises one or more sutures disposed on the cusp edge portion of at least one side of each leaflet of the plurality of leaflets and a plurality of in-and-out stitches extending through the suture and the cusp edge portion of each leaflet; attaching pairs of commissure tabs of adjacent leaflets of the leaflet assembly to commissure windows of the frame by extending each pair of commissure tabs radially through a corresponding commissure window formed by a corresponding commissure support portion of the frame, wherein the plurality of in-and-out stitches further extend along each commissure tab of each pair of commissure tabs; forming a wedge member between commissure tabs of each pair of commissure tabs from two or more overlapping portions of the one or more sutures of the connecting suture line; and securing each wedge member between the commissure tabs of each corresponding pair of commissure tabs with a plurality of stitches extending through the wedge member and the plurality of in-and-out stitches along an inner surface of each commissure tab of the corresponding pair of commissure tabs to form a commissure.

Example 62. The method of any example herein, particularly example 61, wherein the one or more sutures of the connecting suture line comprises a first suture on the cusp edge portion of a first side of each leaflet and a second suture on the cusp edge portion of a second side of each leaflet, and wherein forming the wedge member includes forming the wedge member from two or more overlapping portions of one or more of the first suture and the second suture.

Example 63. The method of any example herein, particularly example 62, wherein a different first suture is disposed on the cusp edge portion of the first side of each leaflet, wherein the second suture extends along the cusp edge portion of the second side of all leaflets of the leaflet assembly, and wherein forming the wedge member includes forming the wedge member from two suture end portions created from the first suture of two adjacent leaflets.

Example 64. The method of any example herein, particularly example 61, wherein the one or more sutures of the connecting suture line comprises three individual first sutures, each individual first suture folded over a first end of the cusp edge portion of a corresponding leaflet of the plurality of leaflets with a first portion of the individual first suture extending along a first side of the corresponding leaflet and a second portion of the individual first suture extending a long an opposite, second side of the corresponding leaflet, and wherein forming the wedge member includes forming the wedge member from two suture end portions formed by free end portions of the first portion and the second portion of the individual first suture at a second end of the cusp edge portion of the corresponding leaflet.

Example 65. The method of any example herein, particularly any one of examples 61-64, wherein a first suture end portion and a second suture end portion are created from the one or more sutures of each leaflet of the connecting suture line at each pair of commissure tabs, and wherein forming the wedge member includes forming the wedge member between the commissure tabs of each pair of commissure tabs from a corresponding first suture end portion and second suture end portion.

Example 66. The method of any example herein, particularly example 65, wherein forming the wedge member, for each pair of commissure tabs, includes extending the first suture end portion from inside the frame and below an inflow end of the corresponding commissure window, across and between the commissure tabs of the corresponding pair of commissure tabs, and to an outflow end of the commissure window, wrapping the first suture end portion around the outflow end of the commissure window, and extending the first suture end portion back across the commissure tabs, between the commissure tabs, thereby forming two suture segments between the commissure tabs of the corresponding pair of commissure tabs.

Example 67. The method of any example herein, particularly example 66, wherein forming the wedge member further includes extending the second suture end portion from inside the frame and below the inflow end of the commissure window, across and between the commissure tabs, toward the outflow end of the commissure window, thereby forming a third suture segment overlapping the two suture segments and a wedge member comprising three suture segments.

Example 68. The method of any example herein, particularly example 67, wherein securing each wedge member between the commissure tabs of each pair of commissure tabs with the plurality of stitches includes extending the plurality of stitches through one or more suture segments of the three suture segments of the wedge member and the plurality of in-and-out stitches along the inner surface of each commissure tab of the pair of commissure tabs.

Example 69. The method of any example herein, particularly any one of examples 61-68, wherein securing each wedge member between the commissure tabs of each pair of commissure tabs with the plurality of stitches includes, for each commissure, forming a plurality of tightened loops along a length of the commissure tabs of the corresponding pair of commissure tabs with a first suture and a second suture, and wherein forming the plurality of loops includes passing the first suture through the wedge member and between a first stitch of the plurality of in-and-out stitches of a first commissure tab of the corresponding pair of commissure tabs and the first commissure tab and passing the second suture through the wedge member and between a second stitch of the plurality of in-and-out stitches of a second commissure tab of the corresponding pair of commissure tabs and the second commissure tab, the first and second stitches disposed opposite one another across the wedge member.

Example 70. The method of any example herein, particularly any one of examples 61-69, further comprising closing the commissure tabs of each pair of commissure tabs around the wedge member by tying strands of a suture used to form the plurality of stitches around outer surfaces of the commissure tabs.

Example 71. The method of any example herein, particularly any one of examples 70, further comprising extending another plurality of stitches through the in-and-out stitches on outer surfaces of the commissure tabs and around the pair of commissure tabs and passing strands of the suture through an interior of the formed commissure.

Example 72. The method of any example herein, particularly any one of examples 61-71, wherein the plurality of in-and-out stitches extend along an edge of each commissure tab of each pair of commissure tabs, wherein the edge is an attached edge of the commissure tab that is connected to a body of a corresponding leaflet.

Example 73. The method of any example herein, particularly any one of examples 61-72, wherein the attaching the connecting suture line to the struts of the frame includes extending a plurality of whip stitches around or through at least a portion of the connecting suture line and around selected struts of the frame to secure the cusp edge portions of the leaflets directly to the frame without extending the whip stitches through a tissue of the leaflets.

Example 74. A prosthetic heart valve comprising: a frame comprising a plurality of interconnected struts and a plurality of commissure supports defining respective commissure windows; a plurality of leaflets, each leaflet comprising opposing commissure tabs disposed on opposite sides of the leaflet and a cusp edge portion extending between the opposing commissure tabs, wherein the plurality of leaflets are connected together and to the frame by a connecting suture line extending along the cusp edge portions of the leaflets, the connecting suture line comprising one or more first sutures arranged along the cusp edge portion on at least one side of each leaflet and one or more second sutures stitched in an in-and-out pattern through the leaflets and the one or more first sutures; and a plurality of commissures secured to the plurality of commissure supports, each commissure comprising two commissure tabs, one from each of two adjacent leaflets of the plurality of leaflets, extending through a corresponding commissure window and exterior to the frame, and a wedge member extending between and along inner surfaces of the two commissure tabs, the wedge member comprising two or more overlapping suture segments of the one or more first sutures of the connecting suture line, and wherein the wedge member is secured to the two commissure tabs with a plurality of stitches extending through the wedge member and a plurality of in-and-out stitches that extend along the inner surfaces of the two commissure tabs.

Example 75. The prosthetic heart valve of any example herein, particularly example 74, wherein each commissure is secured to a corresponding commissure support forming the corresponding commissure window both above and below the commissure window.

Example 76. The prosthetic heart valve of any example herein, particularly any one of examples 74 or example 75, wherein the plurality of in-and-out stitches along the two commissure tabs are continuous with the one or more second sutures of the connecting suture line.

Example 77. The prosthetic heart valve of any example herein, particularly any one of examples 74-76, wherein the two commissure tabs of each commissure are closed around the wedge member by one or more ties formed from strands of one or more sutures that are used to form the plurality of stitches extending through the wedge member and the plurality of in-and-out stitches, wherein the one or more ties loop around outer surfaces of the two commissure tabs.

Example 78. The prosthetic heart valve of any example herein, particularly any one of examples 74-77, wherein the plurality of stitches extending through the wedge member and the plurality of in-and-out stitches extend along a length of the two commissure tabs, between and inflow end and outflow end of the two commissure tabs.

Example 79. The prosthetic heart valve of any example herein, particularly any one of examples 74-78, wherein the plurality of stitches extending through the wedge member and the plurality of in-and-out stitches form a plurality of loops extending along a length of the two commissure tabs, each loop of the plurality of loops extending through opposing stitches of the plurality of in-and-out stitches in the two commissure tabs, the opposing stitches disposed on opposite sides of the wedge member.

Example 80. The prosthetic heart valve of any example herein, particularly any one of examples 74-79, wherein the one or more first sutures have a first thickness and the one or more second sutures have a second thickness, the first thickness greater than the second thickness.

Example 81. The prosthetic heart valve of any example herein, particularly any one of examples 74-80, wherein the one or more first sutures comprises two first sutures disposed on opposite sides of the cusp edge portion of each leaflet, and the one or more second sutures extend through the two first sutures and the cusp edge portion of each leaflet.

Example 82. The prosthetic heart valve of any example herein, particularly any one of examples 74-80, wherein the one or more first sutures comprises a single first suture arranged along the cusp edge portions on a first side of all leaflets of the plurality of leaflets and individual first sutures, each arranged along the cusp edge portion of a second side of a corresponding leaflet of the plurality of leaflets, and wherein the one or more second sutures extend through the single first suture, the individual first sutures, and the cusp edge portion of each leaflet.

Example 83. A prosthetic heart valve comprising: a frame comprising a plurality of interconnected struts and a plurality of commissure supports defining respective commissure windows; a plurality of leaflets, each leaflet comprising opposing commissure tabs disposed on opposite sides of the leaflet and a cusp edge portion extending between the opposing commissure tabs, wherein the plurality of leaflets are connected together and to the frame by a connecting suture line extending along the cusp edge portions of the leaflets, the connecting suture line comprising a plurality of first sutures arranged along the cusp edge portion on both sides of each leaflet and one or more second sutures stitched in an in-and-out pattern through the leaflets and the plurality of first sutures; and a plurality of commissures secured to the plurality of commissure supports, each commissure comprising two commissure tabs, one from each of two adjacent leaflets of the plurality of leaflets, extending through a corresponding commissure window, and a wedge member extending between and along the two commissure tabs, the wedge member formed from two end portions of the plurality of first sutures and comprising three or more overlapping segments of the two end portions.

Example 84. The prosthetic heart valve of any example herein, particularly example 83, wherein the plurality of first sutures includes a single first suture arranged along the cusp edge portions on a first side of all leaflets of the plurality of leaflets and three individual first sutures, each of the three individual first sutures arranged along the cusp edge portion of a second side of a corresponding leaflet of the plurality of leaflets, and wherein the two end portions for the wedge member are formed by free end portions of adjacent individual first sutures of the three individual first sutures.

Example 85. The prosthetic heart valve of any example herein, particularly example 83, wherein the plurality of first sutures includes three individual first sutures, each individual first suture folded over a first end of the cusp edge portion of a corresponding leaflet with a first portion of the individual first suture extending along a first side of the corresponding leaflet and a second portion of the individual first suture extending a long an opposite, second side of the corresponding leaflet, and wherein the two end portions for the wedge member are formed by free end portions of the first portion and the second portion of the individual first suture at a second end of the cusp edge portion of the corresponding leaflet.

Example 86. The prosthetic heart valve of any example herein, particularly either example 84 or example 85, wherein the two end portions of the plurality of first stitches are stitched together to form a thicker portion of the wedge member comprising a plurality of in-and-out stitches with a remainder of the two end portions extending from the thicker portion toward an outflow end of the frame.

Example 87. The prosthetic heart valve of any example herein, particularly any one of examples 83-86, wherein the wedge member extends between the two commissure tabs on an exterior of a corresponding commissure support of the frame, and wherein the two end portions forming the wedge member are secured both above and below the commissure window.

Example 88. The prosthetic heart valve of any example herein, particularly example 87, wherein the two end portions are tied together around an outflow end of the corresponding commissure support, above the commissure window, and wherein only one of the two end portions extends back across the two commissure tabs and is tied around an inflow end of the corresponding commissure support, below the commissure window.

Example 89. The prosthetic heart valve of any example herein, particularly any one of examples 83-88, wherein the two commissure tabs of each commissure are closed around the wedge member by one or more stitches that extend through the wedge member and the two commissure tabs and around outer surfaces of the two commissure tabs.

Example 90. The prosthetic heart valve of any example herein, particularly example 89, wherein a fabric post is secured to each of the two commissure tabs, on the outer surfaces of the two commissure tabs, and wherein the one or more stitches extend through the fabric post of each of the two commissure tabs.

Example 91. The prosthetic heart valve of any example herein, particularly any one of examples 83-90, wherein the plurality of first sutures each have a first thickness and the one or more second sutures each have a second thickness, the first thickness greater than the second thickness.

Example 92. A method comprising: attaching a connecting suture line formed along cusp edge portions of a plurality of leaflets of a leaflet assembly to struts of a frame of a prosthetic heart valve, wherein the connecting suture line comprises one or more sutures disposed on the cusp edge portion of at least one side of each leaflet of the plurality of leaflets and a plurality of in-and-out stitches extending through the suture and the cusp edge portion of each leaflet; attaching pairs of commissure tabs of adjacent leaflets of the leaflet assembly to commissure windows of the frame by extending each pair of commissure tabs radially through a corresponding commissure window formed by a corresponding commissure support portion of the frame; forming a wedge member between commissure tabs of each pair of commissure tabs from two or more overlapping portions of the one or more sutures of the connecting suture line; and securing each wedge member to a first end of the corresponding support portion, above the corresponding commissure window, to a second end of the corresponding support portion, below the corresponding commissure window, and to the commissure tabs to form a commissure.

Example 93. The method of any example herein, particularly example 92, wherein the one or more sutures of the connecting suture line comprises a single first suture arranged along the cusp edge portions on a first side of all leaflets of the plurality of leaflets and three second sutures, each of the second sutures arranged along the cusp edge portion of a second side of a corresponding leaflet of the plurality of leaflets, and wherein forming the wedge member includes forming the wedge member from two suture end portions of two adjacent second sutures.

Example 94. The method of any example herein, particularly example 92, wherein the one or more sutures of the connecting suture line comprises three individual first sutures, each first suture folded over a first end of the cusp edge portion of a corresponding leaflet of the plurality of leaflets with a first portion of the first suture extending along a first side of the corresponding leaflet and a second portion of the first suture extending a long an opposite, second side of the corresponding leaflet, and wherein forming the wedge member includes forming the wedge member from two suture end portions formed by free end portions of the first portion and the second portion of the first suture at a second end of the cusp edge portion of the corresponding leaflet.

Example 95. The method of any example herein, particularly either example 93 or example 94, wherein the two suture end portions comprise a first suture end portion and a second suture end portion, and wherein forming the wedge member, for each pair of commissure tabs, includes extending the first and second suture end portions from inside the frame and below an inflow end of the corresponding commissure window, across and between the commissure tabs of the corresponding pair of commissure tabs, and to an outflow end of the commissure window, and tying the first and second suture end portions together around the outflow end of the commissure window.

Example 96. The method of any example herein, particularly example 95, further comprising extending the first suture end portion back across the commissure tabs, between the commissure tabs, thereby forming three suture segments between the commissure tabs of the corresponding pair of commissure tabs, and tying the first suture end portion around the inflow end of the commissure window.

Example 97. The method of any example herein, particularly any one of examples 92-96, wherein securing each wedge member to the commissure tabs includes extending a plurality of stitches through the wedge member, the commissure tabs, and around outer surfaces of the commissure tabs to close the commissure tabs around the wedge member.

Example 98. The method of any example herein, particularly any one of examples 92-97, wherein the attaching the connecting suture line to the struts of the frame includes extending a plurality of whip stitches around or through at least a portion of the connecting suture line and around selected struts of the frame to secure the cusp edge portions of the leaflets directly to the frame without extending the whip stitches through a tissue of the leaflets.

The features described herein with regard to any example can be combined with other features described in any one or more of the other examples, unless otherwise stated. For example, any one or more of the features of one prosthetic heart valve can be combined with any one or more features of another prosthetic heart valve. As another example, any one or more features of one leaflet assembly can be combined with any one or more features of another leaflet assembly.

In view of the many possible examples to which the principles of the disclosed technology may be applied, it should be recognized that the illustrated examples are only preferred examples of the disclosed technology and should not be taken as limiting the scope of the claimed subject matter. Rather, the scope of the claimed subject matter is defined by the following claims and their equivalents.

We claim:

1. A prosthetic heart valve comprising:
a frame comprising a plurality of interconnected struts;
a plurality of leaflets disposed within the frame and configured to regulate a flow of blood through the frame in one direction, each leaflet comprising a cusp edge portion; and
at least one suture extending along a cusp edge portion of at least a first leaflet of the plurality of leaflets, a plurality of in-and-out stitches extending through the suture and the cusp edge portion of the first leaflet, and a plurality of whip stitches that extend around selected struts of the plurality of the interconnected struts and the suture to couple the cusp edge portion of the first leaflet to the frame.

2. The prosthetic heart valve of claim 1, wherein the at least one suture comprises first and second sutures disposed on opposite sides of the cusp edge portion of the first leaflet, and the in-and-out stitches extend through the first suture, the cusp edge portion of the first leaflet, and the second suture.

3. The prosthetic heart valve of claim 1, wherein the at least one suture comprises a first suture folded over a first end of the cusp edge portion of the first leaflet with a first portion of the first suture extending along a first side of the first leaflet to a second end of the cusp edge portion and a second portion of the first suture extending along an opposite, second side of the first leaflet to the second end of the cusp edge portion, and wherein the in-and-out stitches extend through the first portion of the first suture, the cusp edge portion of the first leaflet, and the second portion of the first suture.

4. The prosthetic heart valve of claim 1, wherein the whip stitches extend between the suture and the cusp edge portion of the first leaflet without extending through the first leaflet.

5. The prosthetic heart valve of claim 1, wherein the whip stitches extend between the suture and the plurality of in-and-out stitches without extending through the first leaflet.

6. The prosthetic heart valve of claim 1, wherein the whip stitches extend through the suture without extending through the first leaflet.

7. The prosthetic heart valve of claim 1, wherein the frame comprises a plurality of commissure supports, and each leaflet comprises opposing commissure tabs, wherein each commissure tab is paired with an adjacent commissure tab of an adjacent leaflet, and each pair of commissure tabs extends radially through an opening in a corresponding commissure support, and wherein the at least one suture forms at least one wedge member that extends between the commissure tabs of at least a first pair of commissure tabs on an outside of the frame.

8. The prosthetic heart valve of claim 7, further comprising a plurality of additional stitches that secure the at least one wedge member to the commissure tabs of the first pair of commissure tabs, and wherein the plurality of additional stitches also secure the at least one wedge member to the corresponding commissure support, above and below the opening in the corresponding commissure support.

9. The prosthetic heart valve of claim 7, wherein the at least one wedge member is formed by two or more overlapping portions of the at least one suture such that a first thickness of the at least one wedge member is thicker than a second thickness of the at least one suture.

10. The prosthetic heart valve of claim 1, wherein the at least one suture is thicker than a thickness of the in-and-out stitches.

11. A prosthetic heart valve comprising:
a frame comprising a plurality of interconnected struts and a plurality of commissure supports defining respective commissure windows;
a plurality of leaflets, each leaflet comprising opposing commissure tabs disposed on opposite sides of the leaflet and a cusp edge portion extending between the opposing commissure tabs, wherein the plurality of leaflets are connected together and to the frame by a connecting suture line extending along the cusp edge portions of the leaflets, the connecting suture line comprising one or more first sutures arranged along the cusp edge portion on at least one side of each leaflet and one or more second sutures stitched in an in-and-out pattern through the leaflets and the one or more first sutures; and
a plurality of commissures secured to the plurality of commissure supports, each commissure comprising two commissure tabs, one from each of two adjacent leaflets of the plurality of leaflets, and a wedge member extending between and along the two commissure tabs on an exterior of a corresponding commissure support of the frame, the wedge member comprising two or more overlapping portions of the one or more first sutures of the connecting suture line.

12. The prosthetic heart valve of claim 11, wherein for each commissure, the two commissure tabs are secured to the wedge member and the corresponding commissure support by one or more third sutures.

13. The prosthetic heart valve of claim 11, wherein the one or more first sutures have a first thickness and the one or more second sutures have a second thickness, the first thickness greater than the second thickness.

14. The prosthetic heart valve of claim 11, wherein the one or more first sutures comprises a single first suture arranged along the cusp edge portions on a first side of all leaflets of the plurality of leaflets and three individual first sutures, each of the three individual first sutures arranged along the cusp edge portion of a second side of a corresponding leaflet of the plurality of leaflets, and wherein the wedge member is formed by free end portions of one of the three individual first sutures.

15. The prosthetic heart valve of claim 11, wherein the one or more first sutures comprises three individual first sutures, each individual first suture folded over a first end of the cusp edge portion of a corresponding leaflet with a first portion of the individual first suture extending along a first side of the corresponding leaflet and a second portion of the individual first suture extending a long an opposite, second side of the corresponding leaflet, and wherein the wedge member is formed by free end portions of the first portion and the second portion of the individual first suture at a second end of the cusp edge portion of the corresponding leaflet.

16. The prosthetic heart valve of claim 11, wherein the cusp edge portion of each leaflet of the plurality of leaflets is attached to selected struts of the plurality of interconnected struts by a plurality of whip stitches that extend around the selected struts and around or through the connecting suture line.

17. The prosthetic heart valve of claim 11, further comprising an outer skirt disposed around an outer surface of the frame, the outer skirt comprising an outflow edge and an inflow edge, the outflow edge attached to the plurality of commissures by one or more stitches that extend through the outer skirt and the wedge member of each commissure.

18. A method comprising:
attaching a connecting suture line formed along cusp edge portions of a plurality of leaflets of a leaflet assembly to struts of a frame of a prosthetic heart valve by extending a plurality of whip stitches around or through at least a portion of the connecting suture line and around selected struts of the frame to secure the cusp edge portions of the leaflets directly to the frame without extending the whip stitches through a tissue of the leaflets, wherein the connecting suture line comprises a suture disposed on the cusp edge portion of at least one side of each leaflet of the plurality of leaflets and a plurality of in-and-out stitches extending through the suture and the cusp edge portion of each leaflet; and
attaching pairs of commissure tabs of adjacent leaflets of the leaflet assembly to commissure windows of the frame by extending each pair of commissure tabs radially through a corresponding commissure window formed by a corresponding commissure support portion of the frame and securing a wedge member integrated with the connecting suture line to the pair of commissure tabs on an exterior of the commissure window, the wedge member formed by two or more overlapping portions of the suture.

19. The method of claim 18, wherein securing the wedge member to the pair of commissure tabs includes extending the wedge member from cusp edge portions of the adjacent leaflets that comprise the pair of commissure tabs, between the pair of commissure tabs, and then securing the pair of commissure tabs to the wedge member and the corresponding commissure support portion.

20. The method of claim 18, further comprising arranging an outer skirt around an outer surface of the frame and securing the outer skirt to the selected struts of the frame with the plurality of whip stitches.
